SEQUENCE LISTING (281 sequences)

SEQ ID NO: 1 - protein, 4 aa, Artificial Sequence
  endomorphin-2
  MOD_RES (4)..(4): modified by NH2
YPFF

SEQ ID NO: 2 - protein, 4 aa, Artificial Sequence
  endomorphin-1
  MOD_RES (4)..(4): modified by NH2
YPWF

SEQ ID NO: 3 - protein, 4 aa, Artificial Sequence
  endomorphin-2 derivative
  MOD_RES (4)..(4): modified by NHCH3
YPFF

SEQ ID NO: 4 - protein, 4 aa, Artificial Sequence
  endomorphin-2 derivative
  MOD_RES (4)..(4): modified by NHCH2CH3
YPFF

SEQ ID NO: 5 - protein, 4 aa, Artificial Sequence
  endomorphin-2 derivative
  MOD_RES (4)..(4): modified by NHCH2CH2CH3
YPFF

SEQ ID NO: 6 - protein, 4 aa, Artificial Sequence
  endomorphin-2 derivative
  MOD_RES (4)..(4): modified by NHCH(CH3)2
YPFF

SEQ ID NO: 7 - protein, 4 aa, Artificial Sequence
  endomorphin-2 derivative
  MOD_RES (4)..(4): NHC6H5
YPFF

SEQ ID NO: 8 - protein, 4 aa, Artificial Sequence
  endomorphin-2 derivative
  MOD_RES (4)..(4): NHCH2C6H5
YPFF

SEQ ID NO: 9 - protein, 4 aa, Artificial Sequence
  endomorphin-2 derivative
  MOD_RES (4)..(4): modified by NHCH2CH2C6H5
YPFF

SEQ ID NO: 10 - protein, 4 aa, Artificial Sequence
  endomorphin-2 derivative
  MOD_RES (4)..(4): modified by NHNH2
YPFF

SEQ ID NO: 11 - protein, 4 aa, Artificial Sequence
  endomorphin-2 derivative
  MOD_RES (4)..(4): modified by NHNHCH3
YPFF

SEQ ID NO: 12 - protein, 4 aa, Artificial Sequence
  endomorphin-2 derivative
  MOD_RES (4)..(4): modified by NHNHCH2CH3
YPFF

SEQ ID NO: 13 - protein, 4 aa, Artificial Sequence
  endomorphin-2 derivative
  MOD_RES (4)..(4): modified by NHNHCH2CH2CH3
YPFF

SEQ ID NO: 14 - protein, 4 aa, Artificial Sequence
  endomorphin-2 derivative
  MOD_RES (4)..(4): modified by NHNHCH(CH3)2
YPFF

SEQ ID NO: 15 - protein, 4 aa, Artificial Sequence
  endomorphin-2 derivative
  MOD_RES (4)..(4): modified by NHNHC6H5
YPFF

SEQ ID NO: 16 - protein, 4 aa, Artificial Sequence
  endomorphin-2 derivative
  MOD_RES (4)..(4): modified by NHNHCH2C6H5
YPFF

SEQ ID NO: 17 - protein, 4 aa, Artificial Sequence
  endomorphin-2 derivative
  MOD_RES (4)..(4): modified by NHNHAc
YPFF

SEQ ID NO: 18 - protein, 4 aa, Artificial Sequence
  endomorphin-2 derivative
  MOD_RES (4)..(4): modified by NHNHPa
YPFF

SEQ ID NO: 19 - protein, 4 aa, Artificial Sequence
  endomorphin-2 derivative
  MOD_RES (4)..(4): modified by NHNHBz
YPFF

SEQ ID NO: 20 - protein, 4 aa, Artificial Sequence
  endomorphin-2 derivative
  MOD_RES (4)..(4): modified by NHNHOa
YPFF

SEQ ID NO: 21 - protein, 4 aa, Artificial Sequence
  endomorphin-2 derivative
  MOD_RES (4)..(4): modified by NHNHFo
YPFF

SEQ ID NO: 22 - protein, 4 aa, Artificial Sequence
  endomorphin-2 derivative
  MOD_RES (4)..(4): modified by NHOH
YPFF

SEQ ID NO: 23 - protein, 4 aa, Artificial Sequence
  endomorphin-2 derivative
  MOD_RES (1)..(1): modified by Ac
  MOD_RES (4)..(4): modified by NH2
YPFF

SEQ ID NO: 24 - protein, 4 aa, Artificial Sequence
  endomorphin-2 derivative
  MOD_RES (1)..(1): modified by Ac
  MOD_RES (4)..(4): modified by NHCH3
YPFF

SEQ ID NO: 25 - protein, 4 aa, Artificial Sequence
  endomorphin-2 derivative
  MOD_RES (1)..(1): modified by Ac
  MOD_RES (4)..(4): modified by NHCH2CH3
YPFF

SEQ ID NO: 26 - protein, 4 aa, Artificial Sequence
  endomorphin-2 derivative
  MOD_RES (1)..(1): modified by Ac
  MOD_RES (4)..(4): modified by NHCH2CH2CH3
YPFF

SEQ ID NO: 27 - protein, 4 aa, Artificial Sequence
  endomorphin-2 derivative
  MOD_RES (1)..(1): modified by Ac
  MOD_RES (4)..(4): modified by NHCH(CH3)2
YPFF

SEQ ID NO: 28 - protein, 4 aa, Artificial Sequence
  endomorphin-2 derivative
  MOD_RES (1)..(1): modified by Ac
  MOD_RES (4)..(4): modified by NHC6H5
YPFF

SEQ ID NO: 29 - protein, 4 aa, Artificial Sequence
  endomorphin-2 derivative
  MOD_RES (1)..(1): modified by Ac
  MOD_RES (4)..(4): modified by NHCH2C6H5
YPFF

SEQ ID NO: 30 - protein, 4 aa, Artificial Sequence
  endomorphin-2 derivative
  MOD_RES (1)..(1): modified by Ac
  MOD_RES (4)..(4): modified by NHCH2CH2C6H5
YPFF

SEQ ID NO: 31 - protein, 4 aa, Artificial Sequence
  endomorphin-2 derivative
  MOD_RES (1)..(1): modified by Ac
  MOD_RES (4)..(4): modified by NHNH2
YPFF

SEQ ID NO: 32 - protein, 4 aa, Artificial Sequence
  endomorphin-2 derivative
  MOD_RES (1)..(1): modified by Ac
  MOD_RES (4)..(4): modified by NHNHCH3
YPFF

SEQ ID NO: 33 - protein, 4 aa, Artificial Sequence
  endomorphin-2 derivative
  MOD_RES (1)..(1): modified by Ac
  MOD_RES (4)..(4): modified by NHNHCH2CH3
YPFF

SEQ ID NO: 34 - protein, 4 aa, Artificial Sequence
  endomorphin-2 derivative
  MOD_RES (1)..(1): modified by Ac
  MOD_RES (4)..(4): modified by NHNHCH2CH2CH3
YPFF

SEQ ID NO: 35 - protein, 4 aa, Artificial Sequence
  endomorphin-2 derivative
  MOD_RES (1)..(1): modified by Ac
  MOD_RES (4)..(4): modified by NHNHCH(CH3)2
YPFF

SEQ ID NO: 36 - protein, 4 aa, Artificial Sequence
  endomorphin-2 derivative
  MOD_RES (1)..(1): modified by Ac
  MOD_RES (4)..(4): modified by NHNHC6H5
YPFF

SEQ ID NO: 37 - protein, 4 aa, Artificial Sequence
  endomorphin-2 derivative
  MOD_RES (1)..(1): modified by Ac
  MOD_RES (4)..(4): modified by Ac NHNHCH2C6H5
YPFF

SEQ ID NO: 38 - protein, 4 aa, Artificial Sequence
  endomorphin-2 derivative
  MOD_RES (1)..(1): modified by Ac
  MOD_RES (4)..(4): modified by NHNHAc
YPFF

SEQ ID NO: 39 - protein, 4 aa, Artificial Sequence
  endomorphin-2 derivative
  MOD_RES (1)..(1): modified by Ac
  MOD_RES (4)..(4): modified by NHNHPa
YPFF

SEQ ID NO: 40 - protein, 4 aa, Artificial Sequence
  endomorphin-2 derivative
  MOD_RES (1)..(1): modified by Ac
  MOD_RES (4)..(4): modified by NHNHBz
YPFF

SEQ ID NO: 41 - protein, 4 aa, Artificial Sequence
  endomorphin-2 derivative
  MOD_RES (1)..(1): modified by Ac
  MOD_RES (4)..(4): modified by NHNHOa
YPFF

SEQ ID NO: 42 - protein, 4 aa, Artificial Sequence
  endomorphin-2 derivative
  MOD_RES (1)..(1): modified by Ac
  MOD_RES (4)..(4): modified by NHNHFo
YPFF

SEQ ID NO: 43 - protein, 4 aa, Artificial Sequence
  endomorphin-2 derivative
  MOD_RES (1)..(1): modified by Ac
  MOD_RES (4)..(4): modified by NHOH
YPFF

SEQ ID NO: 44 - protein, 4 aa, Artificial Sequence
  endomorphin-2 derivative
  MOD_RES (1)..(1): modified by Pg
  MOD_RES (4)..(4): modified by NH2
YPFF

SEQ ID NO: 45 - protein, 4 aa, Artificial Sequence
  endomorphin-2 derivative
  MOD_RES (1)..(1): modified by Pg
  MOD_RES (4)..(4): modified by NHCH3
YPFF

SEQ ID NO: 46 - protein, 4 aa, Artificial Sequence
  endomorphin-2 derivative
  MOD_RES (1)..(1): modified by Pg
  MOD_RES (4)..(4): modified by NHCH2CH3
YPFF

SEQ ID NO: 47 - protein, 4 aa, Artificial Sequence
  endomorphin-2 derivative
  MOD_RES (1)..(1): modified by Pg
  MOD_RES (4)..(4): modified by NHCH2CH2CH3
YPFF

SEQ ID NO: 48 - protein, 4 aa, Artificial Sequence
  endomorphin-2 derivative
  MOD_RES (1)..(1): modified by Pg
  MOD_RES (4)..(4): modified by NHCH(CH3)2
YPFF

SEQ ID NO: 49 - protein, 4 aa, Artificial Sequence
  endomorphin-2 derivative
  MOD_RES (1)..(1): modified by Pg
  MOD_RES (4)..(4): modified by NHC6H5
YPFF

SEQ ID NO: 50 - protein, 4 aa, Artificial Sequence
  endomorphin-2 derivative
  MOD_RES (1)..(1): modified by Pg
  MOD_RES (4)..(4): modified by NHCH2C6H5
YPFF

SEQ ID NO: 51 - protein, 4 aa, Artificial Sequence
  endomorphin-2 derivative
  MOD_RES (1)..(1): modified by Pg
  MOD_RES (4)..(4): modified by NHNH2
YPFF

SEQ ID NO: 52 - protein, 4 aa, Artificial Sequence
  endomorphin-2 derivative
  MOD_RES (1)..(1): modified by Pg
  MOD_RES (4)..(4): modified by NHNHCH3
YPFF

SEQ ID NO: 53 - protein, 4 aa, Artificial Sequence
  endomorphin-2 derivative
  MOD_RES (1)..(1): modified by Pg
  MOD_RES (4)..(4): modified by NHNHCH2CH3
YPFF

SEQ ID NO: 54 - protein, 4 aa, Artificial Sequence
  endomorphin-2 derivative
  MOD_RES (1)..(1): modified by Pg
  MOD_RES (4)..(4): modified by NHNHCH2CH2CH3
YPFF

SEQ ID NO: 55 - protein, 4 aa, Artificial Sequence
  endomorphin-2 derivative
  MOD_RES (1)..(1): modified by Pg
  MOD_RES (4)..(4): modified by NHNHCH(CH3)2
YPFF

SEQ ID NO: 56 - protein, 4 aa, Artificial Sequence
  endomorphin-2 derivative
  MOD_RES (1)..(1): modified by Pg
  MOD_RES (4)..(4): modified by NHNHC6H5
YPFF

SEQ ID NO: 57 - protein, 4 aa, Artificial Sequence
  endomorphin-2 derivative
  MOD_RES (1)..(1): modified by Pg
  MOD_RES (4)..(4): modified by NHNHCH2C6H5
YPFF

SEQ ID NO: 58 - protein, 4 aa, Artificial Sequence
  endomorphin-2 derivative
  MOD_RES (1)..(1): modified by Pg
  MOD_RES (4)..(4): modified by NHNHAc
YPFF

SEQ ID NO: 59 - protein, 4 aa, Artificial Sequence
  endomorphin-2 derivative
  MOD_RES (1)..(1): modified by Pg
  MOD_RES (4)..(4): modified by NHNHPa
YPFF

SEQ ID NO: 60 - protein, 4 aa, Artificial Sequence
  endomorphin-2 derivative
  MOD_RES (1)..(1): modified by Pg
  MOD_RES (4)..(4): modified by NHNHBz
YPFF

SEQ ID NO: 61 - protein, 4 aa, Artificial Sequence
  endomorphin-2 derivative
  MOD_RES (1)..(1): modified by Pg
  MOD_RES (4)..(4): modified by NHNHOa
YPFF

SEQ ID NO: 62 - protein, 4 aa, Artificial Sequence
  endomorphin-2 derivative
  MOD_RES (1)..(1): modified by Pg
  MOD_RES (4)..(4): modified by NHNHFo
YPFF

SEQ ID NO: 63 - protein, 4 aa, Artificial Sequence
  endomorphin-2 derivative
  MOD_RES (1)..(1): modified by Pg
  MOD_RES (4)..(4): modified by NHOH
YPFF

SEQ ID NO: 64 - protein, 4 aa, Artificial Sequence
  endomorphin-2 derivative
  MOD_RES (1)..(1): modified by Pa
  MOD_RES (4)..(4): modified by NH2
YPFF

SEQ ID NO: 65 - protein, 4 aa, Artificial Sequence
  endomorphin-2 derivative
  MOD_RES (1)..(1): modified by Pa
  MOD_RES (4)..(4): modified by NHCH3
YPFF

SEQ ID NO: 66 - protein, 4 aa, Artificial Sequence
  endomorphin-2 derivative
  MOD_RES (1)..(1): modified by Pa
  MOD_RES (4)..(4): modified by NHCH2CH3
YPFF

SEQ ID NO: 67 - protein, 4 aa, Artificial Sequence
  endomorphin-2 derivative
  MOD_RES (1)..(1): modified by Pa
  MOD_RES (4)..(4): modified by NHCH2CH2CH3
YPFF

SEQ ID NO: 68 - protein, 4 aa, Artificial Sequence
  endomorphin-2 derivative
  MOD_RES (1)..(1): modified by Pa
  MOD_RES (4)..(4): modified by NHCH(CH3)2
YPFF

SEQ ID NO: 69 - protein, 4 aa, Artificial Sequence
  endomorphin-2 derivative
  MOD_RES (1)..(1): modified by Pa
  MOD_RES (4)..(4): modified by NHC6H5
YPFF

SEQ ID NO: 70 - protein, 4 aa, Artificial Sequence
  endomorphin-2 derivative
  MOD_RES (1)..(1): modified by Pa
  MOD_RES (4)..(4): modified by NHCH2C6H5
YPFF

SEQ ID NO: 71 - protein, 4 aa, Artificial Sequence
  endomorphin-2 derivative
  MOD_RES (1)..(1): modified by Pa
  MOD_RES (4)..(4): modified by NHNH2
YPFF

SEQ ID NO: 72 - protein, 4 aa, Artificial Sequence
  endomorphin-2 derivative
  MOD_RES (1)..(1): modified by Pa
  MOD_RES (4)..(4): modified by NHNHCH3
YPFF

SEQ ID NO: 73 - protein, 4 aa, Artificial Sequence
  endomorphin-2 derivative
  MOD_RES (1)..(1): modified by Pa
  MOD_RES (4)..(4): modified by NHNHCH2CH3
YPFF

SEQ ID NO: 74 - protein, 4 aa, Artificial Sequence
  endomorphin-2 derivative
  MOD_RES (1)..(1): modified by Pa
  MOD_RES (4)..(4): modified by NHNHCH2CH2CH3
YPFF

SEQ ID NO: 75 - protein, 4 aa, Artificial Sequence
  endomorphin-2 derivative
  MOD_RES (1)..(1): modified by Pa
  MOD_RES (4)..(4): modified by NHNHCH(CH3)2
YPFF

SEQ ID NO: 76 - protein, 4 aa, Artificial Sequence
  endomorphin-2 derivative
  MOD_RES (1)..(1): modified by Pa
  MOD_RES (4)..(4): modified by NHNHC6H5
YPFF

SEQ ID NO: 77 - protein, 4 aa, Artificial Sequence
  endomorphin-2 derivative
  MOD_RES (1)..(1): modified by Pa
  MOD_RES (4)..(4): modified by NHNHCH2C6H5
YPFF

SEQ ID NO: 78 - protein, 4 aa, Artificial Sequence
  endomorphin-2 derivative
  MOD_RES (1)..(1): modified by Pa
  MOD_RES (4)..(4): modified by NHNHAc
YPFF

SEQ ID NO: 79 - protein, 4 aa, Artificial Sequence
  endomorphin-2 derivative
  MOD_RES (1)..(1): modified by Pa
  MOD_RES (4)..(4): modified by NHNHPa
YPFF

SEQ ID NO: 80 - protein, 4 aa, Artificial Sequence
  endomorphin-2 derivative
  MOD_RES (1)..(1): modified by Pa
  MOD_RES (4)..(4): modified by NHNHBz
YPFF

SEQ ID NO: 81 - protein, 4 aa, Artificial Sequence
  endomorphin-2 derivative
  MOD_RES (1)..(1): modified by Pa
  MOD_RES (4)..(4): modified by NHNHOa
YPFF

SEQ ID NO: 82 - protein, 4 aa, Artificial Sequence
  endomorphin-2 derivative
  MOD_RES (1)..(1): modified by Pa
  MOD_RES (4)..(4): modified by NHNHFo
YPFF

SEQ ID NO: 83 - protein, 4 aa, Artificial Sequence
  endomorphin-2 derivative
  MOD_RES (1)..(1): modified by Pa
  MOD_RES (4)..(4): modified by NHOH
YPFF

SEQ ID NO: 84 - protein, 4 aa, Artificial Sequence
  endomorphin-2 derivative
  MOD_RES (1)..(1): modified by Bz
  MOD_RES (4)..(4): modified by NH2
YPFF

SEQ ID NO: 85 - protein, 4 aa, Artificial Sequence
  endomorphin-2 derivative
  MOD_RES (1)..(1): modified by Bz
  MOD_RES (4)..(4): modified by NHCH3
YPFF

SEQ ID NO: 86 - protein, 4 aa, Artificial Sequence
  endomorphin-2 derivative
  MOD_RES (1)..(1): modified by Bz
  MOD_RES (4)..(4): modified by NHCH2CH3
YPFF

SEQ ID NO: 87 - protein, 4 aa, Artificial Sequence
  endomorphin-2 derivative
  MOD_RES (1)..(1): modified by Bz
  MOD_RES (4)..(4): modified by NHCH2CH2CH3
YPFF

SEQ ID NO: 88 - protein, 4 aa, Artificial Sequence
  endomorphin-2 derivative
  MOD_RES (1)..(1): modified by Bz
  MOD_RES (4)..(4): modified by NHCH(CH3)2
YPFF

SEQ ID NO: 89 - protein, 4 aa, Artificial Sequence
  endomorphin-2 derivative
  MOD_RES (1)..(1): modified by Bz
  MOD_RES (4)..(4): modified by NHC6H5
YPFF

SEQ ID NO: 90 - protein, 4 aa, Artificial Sequence
  endomorphin-2 derivative
  MOD_RES (1)..(1): modified by Bz
  MOD_RES (4)..(4): modified by NHCH2C6H5
YPFF

SEQ ID NO: 91 - protein, 4 aa, Artificial Sequence
  endomorphin-2 derivative
  MOD_RES (1)..(1): modified by Bz
  MOD_RES (4)..(4): modified by NHNH2
YPFF

SEQ ID NO: 92 - protein, 4 aa, Artificial Sequence
  endomorphin-2 derivative
  MOD_RES (1)..(1): modified by Bz
  MOD_RES (4)..(4): modified by NHNHCH3
YPFF

SEQ ID NO: 93 - protein, 4 aa, Artificial Sequence
  endomorphin-2 derivative
  MOD_RES (1)..(1): modified by Bz
  MOD_RES (4)..(4): modified by NHNHCH2CH3
YPFF

SEQ ID NO: 94 - protein, 4 aa, Artificial Sequence
  endomorphin-2 derivative
  MOD_RES (1)..(1): modified by Bz
  MOD_RES (4)..(4): modified by NHNHCH2CH2CH3
YPFF

SEQ ID NO: 95 - protein, 4 aa, Artificial Sequence
  endomorphin-2 derivative
  MOD_RES (1)..(1): modified by Bz
  MOD_RES (4)..(4): modified by NHNHCH(CH3)2
YPFF

SEQ ID NO: 96 - protein, 4 aa, Artificial Sequence
  endomorphin-2 derivative
  MOD_RES (1)..(1): modified by Bz
  MOD_RES (4)..(4): modified by NHNHC6H5
YPFF

SEQ ID NO: 97 - protein, 4 aa, Artificial Sequence
  endomorphin-2 derivative
  MOD_RES (1)..(1): modified by Bz
  MOD_RES (4)..(4): modified by NHNHCH2C6H5
YPFF

SEQ ID NO: 98 - protein, 4 aa, Artificial Sequence
  endomorphin-2 derivative
  MOD_RES (1)..(1): modified by Bz
  MOD_RES (4)..(4): modified by NHNHAc
YPFF

SEQ ID NO: 99 - protein, 4 aa, Artificial Sequence
  endomorphin-2 derivative
  MOD_RES (1)..(1): modified by Bz
  MOD_RES (4)..(4): modified by NHNHPa
YPFF

SEQ ID NO: 100 - protein, 4 aa, Artificial Sequence
  endomorphin-2 derivative
  MOD_RES (1)..(1): modified by Bz
  MOD_RES (4)..(4): modified by NHNHBz
YPFF

SEQ ID NO: 101 - protein, 4 aa, Artificial Sequence
  endomorphin-2 derivative
  MOD_RES (1)..(1): modified by Bz
  MOD_RES (4)..(4): modified by NHNHOa
YPFF

SEQ ID NO: 102 - protein, 4 aa, Artificial Sequence
  endomorphin-2 derivative
  MOD_RES (1)..(1): modified by Bz
  MOD_RES (4)..(4): modified by NHNHFo
YPFF

SEQ ID NO: 103 - protein, 4 aa, Artificial Sequence
  endomorphin-2 derivative
  MOD_RES (1)..(1): modified by Bz
  MOD_RES (4)..(4): modified by NHOH
YPFF

SEQ ID NO: 104 - protein, 4 aa, Artificial Sequence
  endomorphin-2 derivative
  MOD_RES (1)..(1): modified by Fo
  MOD_RES (4)..(4): modified by NH2
YPFF

SEQ ID NO: 105 - protein, 4 aa, Artificial Sequence
  endomorphin-2 derivative
  MOD_RES (1)..(1): modified by Fo
  MOD_RES (4)..(4): modified by NHCH3
YPFF

SEQ ID NO: 106 - protein, 4 aa, Artificial Sequence
  endomorphin-2 derivative
  MOD_RES (1)..(1): modified by Fo
  MOD_RES (4)..(4): modified by NHCH2CH3
YPFF

SEQ ID NO: 107 - protein, 4 aa, Artificial Sequence
  endomorphin-2 derivative
  MOD_RES (1)..(1): modified by Fo
  MOD_RES (4)..(4): modified by NHCH2CH2CH3;
YPFF

SEQ ID NO: 108 - protein, 4 aa, Artificial Sequence
  endomorphin-2 derivative
  MOD_RES (1)..(1): modified by Fo
  MOD_RES (4)..(4): modified by NHCH(CH3)2
YPFF

SEQ ID NO: 109 - protein, 4 aa, Artificial Sequence
  endomorphin-2 derivative
  MOD_RES (1)..(1): modified by Fo
  MOD_RES (4)..(4): modified by NHC6H5
YPFF

SEQ ID NO: 110 - protein, 4 aa, Artificial Sequence
  endomorphin-2 derivative
  MOD_RES (1)..(1): modified by Fo
  MOD_RES (4)..(4): modified by NHCH2C6H5
YPFF

SEQ ID NO: 111 - protein, 4 aa, Artificial Sequence
  endomorphin-2 derivative
  MOD_RES (1)..(1): modified by Fo
  MOD_RES (4)..(4): modified by NHNH2
YPFF

SEQ ID NO: 112 - protein, 4 aa, Artificial Sequence
  endomorphin-2 derivative
  MOD_RES (1)..(1): modified by Fo
  MOD_RES (4)..(4): modified by NHNHCH3
YPFF

SEQ ID NO: 113 - protein, 4 aa, Artificial Sequence
  endomorphin-2 derivative
  MOD_RES (1)..(1): modified by Fo
  MOD_RES (4)..(4): modified by NHNHCH2CH3
YPFF

SEQ ID NO: 114 - protein, 4 aa, Artificial Sequence
  endomorphin-2 derivative
  MOD_RES (1)..(1): modified by Fo
  MOD_RES (4)..(4): modified by NHNHCH2CH2CH3
YPFF

SEQ ID NO: 115 - protein, 4 aa, Artificial Sequence
  endomorphin-2 derivative
  MOD_RES (1)..(1): modified by Fo
  MOD_RES (4)..(4): modified by NHNHCH(CH3)2
YPFF

SEQ ID NO: 116 - protein, 4 aa, Artificial Sequence
  endomorphin-2 derivative
  MOD_RES (1)..(1): modified by Fo
  MOD_RES (4)..(4): modified by NHNHC6H5
YPFF

SEQ ID NO: 117 - protein, 4 aa, Artificial Sequence
  endomorphin-2 derivative
  MOD_RES (1)..(1): modified by Fo
  MOD_RES (4)..(4): modified by NHNHCH2C6H5
YPFF

SEQ ID NO: 118 - protein, 4 aa, Artificial Sequence
  endomorphin-2 derivative
  MOD_RES (1)..(1): modified by Fo
  MOD_RES (4)..(4): modified by NHNHAc
YPFF

SEQ ID NO: 119 - protein, 4 aa, Artificial Sequence
  endomorphin-2 derivative
  MOD_RES (1)..(1): modified by Fo
  MOD_RES (4)..(4): modified by NHNHPa
YPFF

SEQ ID NO: 120 - protein, 4 aa, Artificial Sequence
  endomorphin-2 derivative
  MOD_RES (1)..(1): modified by Fo
  MOD_RES (4)..(4): modified by NHNHBz
YPFF

SEQ ID NO: 121 - protein, 4 aa, Artificial Sequence
  endomorphin-2 derivative
  MOD_RES (1)..(1): modified by Fo
  MOD_RES (4)..(4): modified by NHNHOa
YPFF

SEQ ID NO: 122 - protein, 4 aa, Artificial Sequence
  endomorphin-2 derivative
  MOD_RES (1)..(1): modified by Fo
  MOD_RES (4)..(4): modified by NHNHFo
YPFF

SEQ ID NO: 123 - protein, 4 aa, Artificial Sequence
  endomorphin-2 derivative
  MOD_RES (1)..(1): modified by Fo
  MOD_RES (4)..(4): modified by NHOH
YPFF

SEQ ID NO: 124 - protein, 4 aa, Artificial Sequence
  endomorphin-2 derivative
  MOD_RES (1)..(1): modified by Ab
  MOD_RES (4)..(4): modified by NH2
YPFF

SEQ ID NO: 125 - protein, 4 aa, Artificial Sequence
  endomorphin-2 derivative
  MOD_RES (1)..(1): modified by Ab
  MOD_RES (4)..(4): modified by NHCH3
YPFF

SEQ ID NO: 126 - protein, 4 aa, Artificial Sequence
  endomorphin-2 derivative
  MOD_RES (1)..(1): modified by Ab
  MOD_RES (4)..(4): modified by NHCH2CH3
YPFF

SEQ ID NO: 127 - protein, 4 aa, Artificial Sequence
  endomorphin-2 derivative
  MOD_RES (1)..(1): modified by Ab
  MOD_RES (4)..(4): modified by NHCH2CH2CH3
YPFF

SEQ ID NO: 128 - protein, 4 aa, Artificial Sequence
  endomorphin-2 derivative
  MOD_RES (1)..(1): modified by Ab
  MOD_RES (4)..(4): modified by NHCH(CH3)2
YPFF

SEQ ID NO: 129 - protein, 4 aa, Artificial Sequence
  endomorphin-2 derivative
  MOD_RES (1)..(1): modified by Ab
  MOD_RES (4)..(4): modified by NHC6H5
YPFF

SEQ ID NO: 130 - protein, 4 aa, Artificial Sequence
  endomorphin-2 derivative
  MOD_RES (1)..(1): modified by Ab
  MOD_RES (4)..(4): modified by NHCH2C6H5
YPFF

SEQ ID NO: 131 - protein, 4 aa, Artificial Sequence
  endomorphin-2 derivative
  MOD_RES (1)..(1): modified by Ab
  MOD_RES (4)..(4): modified by NHNH2
YPFF

SEQ ID NO: 132 - protein, 4 aa, Artificial Sequence
  endomorphin-2 derivative
  MOD_RES (1)..(1): modified by Ab
  MOD_RES (4)..(4): modified by NHNHCH3
YPFF

SEQ ID NO: 133 - protein, 4 aa, Artificial Sequence
  endomorphin-2 derivative
  MOD_RES (1)..(1): modified by Ab
  MOD_RES (4)..(4): modified by NHNHCH2CH3
YPFF

SEQ ID NO: 134 - protein, 4 aa, Artificial Sequence
  endomorphin-2 derivative
  MOD_RES (1)..(1): modified by Ab
  MOD_RES (4)..(4): modified by NHNHCH2CH2CH3
YPFF

SEQ ID NO: 135 - protein, 4 aa, Artificial Sequence
  endomorphin-2 derivative
  MOD_RES (1)..(1): modified by Ab
  MOD_RES (4)..(4): modified by NHNHCH(CH3)2
YPFF

SEQ ID NO: 136 - protein, 4 aa, Artificial Sequence
  endomorphin-2 derivative
  MOD_RES (1)..(1): modified by Ab
  MOD_RES (4)..(4): modified by NHNHC6H5
YPFF

SEQ ID NO: 137 - protein, 4 aa, Artificial Sequence
  endomorphin-2 derivative
  MOD_RES (1)..(1): modified by Ab
  MOD_RES (4)..(4): modified by NHNHCH2C6H5
YPFF

SEQ ID NO: 138 - protein, 4 aa, Artificial Sequence
  endomorphin-2 derivative
  MOD_RES (1)..(1): modified by Ab
  MOD_RES (4)..(4): modified by NHNHAc
YPFF

SEQ ID NO: 139 - protein, 4 aa, Artificial Sequence
  endomorphin-2 derivative
  MOD_RES (1)..(1): modified by Ab
  MOD_RES (4)..(4): modified by NHNHPa
YPFF

SEQ ID NO: 140 - protein, 4 aa, Artificial Sequence
  endomorphin-2 derivative
  MOD_RES (1)..(1): modified by Ab
  MOD_RES (4)..(4): modified by NHNHBz
YPFF

SEQ ID NO: 141 - protein, 4 aa, Artificial Sequence
  endomorphin-2 derivative
  MOD_RES (1)..(1): modified by Ab
  MOD_RES (4)..(4): modified by NHNHOa
YPFF

SEQ ID NO: 142 - protein, 4 aa, Artificial Sequence
  endomorphin-2 derivative
  MOD_RES (1)..(1): modified by Ab
  MOD_RES (4)..(4): modified by NHNHFo
YPFF

SEQ ID NO: 143 - protein, 4 aa, Artificial Sequence
  endomorphin-2 derivative
  MOD_RES (1)..(1): modified by Ab
  MOD_RES (4)..(4): modified by NHOH
YPFF

SEQ ID NO: 144 - protein, 4 aa, Artificial Sequence
  endomorphin-2 derivative
  MOD_RES (1)..(1): modifiedy by Bo
  MOD_RES (4)..(4): modifiedy by NH2
YPFF

SEQ ID NO: 145 - protein, 4 aa, Artificial Sequence
  endomorphin-2 derivative
  MOD_RES (1)..(1): modifiedy by Bo
  MOD_RES (4)..(4): modifiedy by NHCH3
YPFF

SEQ ID NO: 146 - protein, 4 aa, Artificial Sequence
  endomorphin-2 derivative
  MOD_RES (1)..(1): modifiedy by Bo
  MOD_RES (4)..(4): modifiedy by NHCH2CH3
YPFF

SEQ ID NO: 147 - protein, 4 aa, Artificial Sequence
  endomorphin-2 derivative
  MOD_RES (1)..(1): modifiedy by Bo
  MOD_RES (4)..(4): modifiedy by NHCH2CH2CH3
YPFF

SEQ ID NO: 148 - protein, 4 aa, Artificial Sequence
  endomorphin-2 derivative
  MOD_RES (1)..(1): modifiedy by Bo
  MOD_RES (4)..(4): modifiedy by NHCH(CH3)2
YPFF

SEQ ID NO: 149 - protein, 4 aa, Artificial Sequence
  endomorphin-2 derivative
  MOD_RES (1)..(1): modifiedy by Bo
  MOD_RES (4)..(4): modifiedy by NHC6H5
YPFF

SEQ ID NO: 150 - protein, 4 aa, Artificial Sequence
  endomorphin-2 derivative
  MOD_RES (1)..(1): modifiedy by Bo
  MOD_RES (4)..(4): modifiedy by NHCH2C6H5
YPFF

SEQ ID NO: 151 - protein, 4 aa, Artificial Sequence
  endomorphin-2 derivative
  MOD_RES (1)..(1): modifiedy by Bo
  MOD_RES (4)..(4): modifiedy by NHNH2
YPFF

SEQ ID NO: 152 - protein, 4 aa, Artificial Sequence
  endomorphin-2 derivative
  MOD_RES (1)..(1): modifiedy by Bo
  MOD_RES (4)..(4): modifiedy by NHNHCH3
YPFF

SEQ ID NO: 153 - protein, 4 aa, Artificial Sequence
  endomorphin-2 derivative
  MOD_RES (1)..(1): modifiedy by Bo
  MOD_RES (4)..(4): modifiedy by NHNHCH2CH3
YPFF

SEQ ID NO: 154 - protein, 4 aa, Artificial Sequence
  endomorphin-2 derivative
  MOD_RES (1)..(1): modifiedy by Bo
  MOD_RES (4)..(4): modifiedy by NHNHCH2CH2CH3
YPFF

SEQ ID NO: 155 - protein, 4 aa, Artificial Sequence
  endomorphin-2 derivative
  MOD_RES (1)..(1): modifiedy by Bo
  MOD_RES (4)..(4): modifiedy by NHNHCH(CH3)2
YPFF

SEQ ID NO: 156 - protein, 4 aa, Artificial Sequence
  endomorphin-2 derivative
  MOD_RES (1)..(1): modifiedy by Bo
  MOD_RES (4)..(4): modifiedy by NHNHC6H5
YPFF

SEQ ID NO: 157 - protein, 4 aa, Artificial Sequence
  endomorphin-2 derivative
  MOD_RES (1)..(1): modifiedy by Bo
  MOD_RES (4)..(4): modifiedy by NHNHCH2C6H5
YPFF

SEQ ID NO: 158 - protein, 4 aa, Artificial Sequence
  endomorphin-2 derivative
  MOD_RES (1)..(1): modifiedy by Bo
  MOD_RES (4)..(4): modifiedy by NHNHAc
YPFF

SEQ ID NO: 159 - protein, 4 aa, Artificial Sequence
  endomorphin-2 derivative
  MOD_RES (1)..(1): modifiedy by Bo
  MOD_RES (4)..(4): modifiedy by NHNHPa
YPFF

SEQ ID NO: 160 - protein, 4 aa, Artificial Sequence
  endomorphin-2 derivative
  MOD_RES (1)..(1): modifiedy by Bo
  MOD_RES (4)..(4): modifiedy by NHNHBz
YPFF

SEQ ID NO: 161 - protein, 4 aa, Artificial Sequence
  endomorphin-2 derivative
  MOD_RES (1)..(1): modifiedy by Bo
  MOD_RES (4)..(4): modifiedy by NHNHOa
YPFF

SEQ ID NO: 162 - protein, 4 aa, Artificial Sequence
  endomorphin-2 derivative
  MOD_RES (1)..(1): modifiedy by Bo
  MOD_RES (4)..(4): modifiedy by NHNHFo
YPFF

SEQ ID NO: 163 - protein, 4 aa, Artificial Sequence
  endomorphin-2 derivative
  MOD_RES (1)..(1): modifiedy by Bo
  MOD_RES (4)..(4): modifiedy by NHOH
YPFF

SEQ ID NO: 164 - protein, 4 aa, Artificial Sequence
  endomorphin-2 derivative
  MOD_RES (1)..(1): modifiedy by Hd
  MOD_RES (4)..(4): modifiedy by NH2
YPFF

SEQ ID NO: 165 - protein, 4 aa, Artificial Sequence
  endomorphin-2 derivative
  MOD_RES (1)..(1): modifiedy by Hd
  MOD_RES (4)..(4): modifiedy by NHCH3
YPFF

SEQ ID NO: 166 - protein, 4 aa, Artificial Sequence
  endomorphin-2 derivative
  MOD_RES (1)..(1): modifiedy by Hd
  MOD_RES (4)..(4): modifiedy by NHCH2CH3
YPFF

SEQ ID NO: 167 - protein, 4 aa, Artificial Sequence
  endomorphin-2 derivative
  MOD_RES (1)..(1): modifiedy by Hd
  MOD_RES (4)..(4): modifiedy by NHCH2CH2CH3
YPFF

SEQ ID NO: 168 - protein, 4 aa, Artificial Sequence
  endomorphin-2 derivative
  MOD_RES (1)..(1): modifiedy by Hd
  MOD_RES (4)..(4): modifiedy by NHCH(CH3)2
YPFF

SEQ ID NO: 169 - protein, 4 aa, Artificial Sequence
  endomorphin-2 derivative
  MOD_RES (1)..(1): modifiedy by Hd
  MOD_RES (4)..(4): modifiedy by NHC6H5
YPFF

SEQ ID NO: 170 - protein, 4 aa, Artificial Sequence
  endomorphin-2 derivative
  MOD_RES (1)..(1): modifiedy by Hd
  MOD_RES (4)..(4): modifiedy by NHCH2C6H5
YPFF

SEQ ID NO: 171 - protein, 4 aa, Artificial Sequence
  endomorphin-2 derivative
  MOD_RES (1)..(1): modifiedy by Hd
  MOD_RES (4)..(4): modifiedy by NHCH2CH2C6H5
YPFF

SEQ ID NO: 172 - protein, 4 aa, Artificial Sequence
  endomorphin-2 derivative
  MOD_RES (1)..(1): modifiedy by Hd
  MOD_RES (4)..(4): modifiedy by NHNH2
YPFF

SEQ ID NO: 173 - protein, 4 aa, Artificial Sequence
  endomorphin-2 derivative
  MOD_RES (1)..(1): modifiedy by Hd
  MOD_RES (4)..(4): modifiedy by NHNHCH3
YPFF

SEQ ID NO: 174 - protein, 4 aa, Artificial Sequence
  endomorphin-2 derivative
  MOD_RES (1)..(1): modifiedy by Hd
  MOD_RES (4)..(4): modifiedy by NHNHCH2CH3
YPFF

SEQ ID NO: 175 - protein, 4 aa, Artificial Sequence
  endomorphin-2 derivative
  MOD_RES (1)..(1): modifiedy by Hd
  MOD_RES (4)..(4): modifiedy by NHNHCH2CH2CH3
YPFF

SEQ ID NO: 176 - protein, 4 aa, Artificial Sequence
  endomorphin-2 derivative
  MOD_RES (1)..(1): modifiedy by Hd
  MOD_RES (4)..(4): modifiedy by NHNHCH(CH3)2
YPFF

SEQ ID NO: 177 - protein, 4 aa, Artificial Sequence
  endomorphin-2 derivative
  MOD_RES (1)..(1): modifiedy by Hd
  MOD_RES (4)..(4): modifiedy by NHNHC6H5
YPFF

SEQ ID NO: 178 - protein, 4 aa, Artificial Sequence
  endomorphin-2 derivative
  MOD_RES (1)..(1): modifiedy by Hd
  MOD_RES (4)..(4): modifiedy by NHNHCH2C6H5
YPFF

SEQ ID NO: 179 - protein, 4 aa, Artificial Sequence
  endomorphin-2 derivative
  MOD_RES (1)..(1): modifiedy by Hd
  MOD_RES (4)..(4): modifiedy by NHNHAc
YPFF

SEQ ID NO: 180 - protein, 4 aa, Artificial Sequence
  endomorphin-2 derivative
  MOD_RES (1)..(1): modifiedy by Hd
  MOD_RES (4)..(4): modifiedy by NHNHPa
YPFF

SEQ ID NO: 181 - protein, 4 aa, Artificial Sequence
  endomorphin-2 derivative
  MOD_RES (1)..(1): modifiedy by Hd
  MOD_RES (4)..(4): modifiedy by NHNHBz
YPFF

SEQ ID NO: 182 - protein, 4 aa, Artificial Sequence
  endomorphin-2 derivative
  MOD_RES (1)..(1): modifiedy by Hd
  MOD_RES (4)..(4): modifiedy by NHOH
YPFF

SEQ ID NO: 183 - protein, 4 aa, Artificial Sequence
  endomorphin-2 derivative
  MOD_RES (1)..(1): modifiedy by Pc
  MOD_RES (4)..(4): modifiedy by NH2
YPFF

SEQ ID NO: 184 - protein, 4 aa, Artificial Sequence
  endomorphin-2 derivative
  MOD_RES (1)..(1): modifiedy by Pc
  MOD_RES (4)..(4): modifiedy by NHCH3
YPFF

SEQ ID NO: 185 - protein, 4 aa, Artificial Sequence
  endomorphin-2 derivative
  MOD_RES (1)..(1): modifiedy by Pc
  MOD_RES (4)..(4): modifiedy by NHCH2CH3
YPFF

SEQ ID NO: 186 - protein, 4 aa, Artificial Sequence
  endomorphin-2 derivative
  MOD_RES (1)..(1): modifiedy by Pc
  MOD_RES (4)..(4): modifiedy by NHCH2CH2CH3
YPFF

SEQ ID NO: 187 - protein, 4 aa, Artificial Sequence
  endomorphin-2 derivative
  MOD_RES (1)..(1): modifiedy by Pc
  MOD_RES (4)..(4): modifiedy by NHCH(CH3)2
YPFF

SEQ ID NO: 188 - protein, 4 aa, Artificial Sequence
  endomorphin-2 derivative
  MOD_RES (1)..(1): modifiedy by Pc
  MOD_RES (4)..(4): modifiedy by NHC6H5
YPFF

SEQ ID NO: 189 - protein, 4 aa, Artificial Sequence
  endomorphin-2 derivative
  MOD_RES (1)..(1): modifiedy by Pc
  MOD_RES (4)..(4): modifiedy by NHCH2C6H5
YPFF

SEQ ID NO: 190 - protein, 4 aa, Artificial Sequence
  endomorphin-2 derivative
  MOD_RES (1)..(1): modifiedy by Pc
  MOD_RES (4)..(4): modifiedy by NHCH2CH2C6H5
YPFF

SEQ ID NO: 191 - protein, 4 aa, Artificial Sequence
  endomorphin-2 derivative
  MOD_RES (1)..(1): modifiedy by Pc
  MOD_RES (4)..(4): modifiedy by NHNH2
YPFF

SEQ ID NO: 192 - protein, 4 aa, Artificial Sequence
  endomorphin-2 derivative
  MOD_RES (1)..(1): modifiedy by Pc
  MOD_RES (4)..(4): modifiedy by NHNHCH3
YPFF

SEQ ID NO: 193 - protein, 4 aa, Artificial Sequence
  endomorphin-2 derivative
  MOD_RES (1)..(1): modifiedy by Pc
  MOD_RES (4)..(4): modifiedy by NHNHCH2CH3
YPFF

SEQ ID NO: 194 - protein, 4 aa, Artificial Sequence
  endomorphin-2 derivative
  MOD_RES (1)..(1): modifiedy by Pc
  MOD_RES (4)..(4): modifiedy by NHNHCH2CH2CH3
YPFF

SEQ ID NO: 195 - protein, 4 aa, Artificial Sequence
  endomorphin-2 derivative
  MOD_RES (1)..(1): modifiedy by Pc
  MOD_RES (4)..(4): modifiedy by NHNHCH(CH3)2
YPFF

SEQ ID NO: 196 - protein, 4 aa, Artificial Sequence
  endomorphin-2 derivative
  MOD_RES (1)..(1): modifiedy by Pc
  MOD_RES (4)..(4): modifiedy by NHNHC6H5
YPFF

SEQ ID NO: 197 - protein, 4 aa, Artificial Sequence
  endomorphin-2 derivative
  MOD_RES (1)..(1): modifiedy by Pc
  MOD_RES (4)..(4): modifiedy by NHNHCH2C6H5
YPFF

SEQ ID NO: 198 - protein, 4 aa, Artificial Sequence
  endomorphin-2 derivative
  MOD_RES (1)..(1): modifiedy by Pc
  MOD_RES (4)..(4): modifiedy by NHNHAc
YPFF

SEQ ID NO: 199 - protein, 4 aa, Artificial Sequence
  endomorphin-2 derivative
  MOD_RES (1)..(1): modifiedy by Pc
  MOD_RES (4)..(4): modifiedy by NHNHPa
YPFF

SEQ ID NO: 200 - protein, 4 aa, Artificial Sequence
  endomorphin-2 derivative
  MOD_RES (1)..(1): modifiedy by Pc
  MOD_RES (4)..(4): modifiedy by NHNHBz
YPFF

SEQ ID NO: 201 - protein, 4 aa, Artificial Sequence
  endomorphin-2 derivative
  MOD_RES (1)..(1): modifiedy by Pc
  MOD_RES (4)..(4): modifiedy by NHOH
YPFF

SEQ ID NO: 202 - protein, 4 aa, Artificial Sequence
  endomorphin-2 derivative
  MOD_RES (1)..(1): modifiedy by Ba
  MOD_RES (4)..(4): modifiedy by NH2
YPFF

SEQ ID NO: 203 - protein, 4 aa, Artificial Sequence
  endomorphin-2 derivative
  MOD_RES (1)..(1): modifiedy by Pe
  MOD_RES (4)..(4): modifiedy by NH2
YPFF

SEQ ID NO: 204 - protein, 4 aa, Artificial Sequence
  endomorphin-2 derivative
  MOD_RES (1)..(1): modifiedy by He
  MOD_RES (4)..(4): modifiedy by NH2
YPFF

SEQ ID NO: 205 - protein, 4 aa, Artificial Sequence
  endomorphin-2 derivative
  MOD_RES (1)..(1): modifiedy by Hp
  MOD_RES (4)..(4): modifiedy by NH2
YPFF

SEQ ID NO: 206 - protein, 4 aa, Artificial Sequence
  endomorphin-2 derivative
  MOD_RES (1)..(1): modifiedy by Oa
  MOD_RES (4)..(4): modifiedy by NH2
YPFF

SEQ ID NO: 207 - protein, 4 aa, Artificial Sequence
  endomorphin-2 derivative
  MOD_RES (1)..(1): modifiedy by Na
  MOD_RES (4)..(4): modifiedy by NH2
YPFF

SEQ ID NO: 208 - protein, 4 aa, Artificial Sequence
  endomorphin-2 derivative
  MOD_RES (1)..(1): modifiedy by Ba
  MOD_RES (4)..(4): modifiedy by NHNH2
YPFF

SEQ ID NO: 209 - protein, 4 aa, Artificial Sequence
  endomorphin-2 derivative
  MOD_RES (1)..(1): modifiedy by Pe
  MOD_RES (4)..(4): modifiedy by NHNH2
YPFF

SEQ ID NO: 210 - protein, 4 aa, Artificial Sequence
  endomorphin-2 derivative
  MOD_RES (1)..(1): modifiedy by He
  MOD_RES (4)..(4): modifiedy by NHNH2
YPFF

SEQ ID NO: 211 - protein, 4 aa, Artificial Sequence
  endomorphin-2 derivative
  MOD_RES (1)..(1): modifiedy by Hp
  MOD_RES (4)..(4): modifiedy by NHNH2
YPFF

SEQ ID NO: 212 - protein, 4 aa, Artificial Sequence
  endomorphin-2 derivative
  MOD_RES (1)..(1): modifiedy by Oa
  MOD_RES (4)..(4): modifiedy by NHNH2
YPFF

SEQ ID NO: 213 - protein, 4 aa, Artificial Sequence
  endomorphin-2 derivative
  MOD_RES (1)..(1): modifiedy by Na
  MOD_RES (4)..(4): modifiedy by NHNH2
YPFF

SEQ ID NO: 214 - protein, 4 aa, Artificial Sequence
  endomorphin-2 derivative
  MOD_RES (1)..(1): modifiedy by Ba
  MOD_RES (4)..(4): modifiedy by NHOH
YPFF

SEQ ID NO: 215 - protein, 4 aa, Artificial Sequence
  endomorphin-2 derivative
  MOD_RES (1)..(1): modifiedy by Pe
  MOD_RES (4)..(4): modifiedy by NHOH
YPFF

SEQ ID NO: 216 - protein, 4 aa, Artificial Sequence
  endomorphin-2 derivative
  MOD_RES (1)..(1): modifiedy by He
  MOD_RES (4)..(4): modifiedy by NHOH
YPFF

SEQ ID NO: 217 - protein, 4 aa, Artificial Sequence
  endomorphin-2 derivative
  MOD_RES (1)..(1): modifiedy by Hp
  MOD_RES (4)..(4): modifiedy by NHOH
YPFF

SEQ ID NO: 218 - protein, 4 aa, Artificial Sequence
  endomorphin-2 derivative
  MOD_RES (1)..(1): modifiedy by Oa
  MOD_RES (4)..(4): modifiedy by NHOH
YPFF

SEQ ID NO: 219 - protein, 4 aa, Artificial Sequence
  endomorphin-2 derivative
  MOD_RES (1)..(1): modifiedy by Na
  MOD_RES (4)..(4): modifiedy by NHOH
YPFF

SEQ ID NO: 220 - protein, 4 aa, Artificial Sequence
  endomorphin-2 derivative
  MOD_RES (1)..(1): modified by Ip
  MOD_RES (4)..(4): modified by NH2
YPFF

SEQ ID NO: 221 - protein, 4 aa, Artificial Sequence
  endomorphin-2 derivative
  MOD_RES (1)..(1): modified by Le
  MOD_RES (4)..(4): modified by NH2
YPFF

SEQ ID NO: 222 - protein, 4 aa, Artificial Sequence
  endomorphin-2 derivative
  MOD_RES (1)..(1): modified by Ln
  MOD_RES (4)..(4): modified by NH2
YPFF

SEQ ID NO: 223 - protein, 4 aa, Artificial Sequence
  endomorphin-2 derivative
  MOD_RES (1)..(1): modified by Np
  MOD_RES (4)..(4): modified by NH2
YPFF

SEQ ID NO: 224 - protein, 4 aa, Artificial Sequence
  endomorphin-2 derivative
  MOD_RES (1)..(1): modified by Ip
  MOD_RES (4)..(4): modified by NHNH2
YPFF

SEQ ID NO: 225 - protein, 4 aa, Artificial Sequence
  endomorphin-2 derivative
  MOD_RES (1)..(1): modified by Le
  MOD_RES (4)..(4): modified by NHNH2
YPFF

SEQ ID NO: 226 - protein, 4 aa, Artificial Sequence
  endomorphin-2 derivative
  MOD_RES (1)..(1): modified by Ln
  MOD_RES (4)..(4): modified by NHNH2
YPFF

SEQ ID NO: 227 - protein, 4 aa, Artificial Sequence
  endomorphin-2 derivative
  MOD_RES (1)..(1): modified by Np
  MOD_RES (4)..(4): modified by NHNH2
YPFF

SEQ ID NO: 228 - protein, 4 aa, Artificial Sequence
  endomorphin-2 derivative
  MOD_RES (1)..(1): modified by Ba
  MOD_RES (4)..(4): modified by NHNHAc
YPFF

SEQ ID NO: 229 - protein, 4 aa, Artificial Sequence
  endomorphin-2 derivative
  MOD_RES (1)..(1): modified by He
  MOD_RES (4)..(4): modified by NHNHAc
YPFF

SEQ ID NO: 230 - protein, 4 aa, Artificial Sequence
  endomorphin-2 derivative
  MOD_RES (1)..(1): modified by Hp
  MOD_RES (4)..(4): modified by NHNHAc
YPFF

SEQ ID NO: 231 - protein, 4 aa, Artificial Sequence
  endomorphin-2 derivative
  MOD_RES (1)..(1): modified by Ip
  MOD_RES (4)..(4): modified by NHNHAc
YPFF

SEQ ID NO: 232 - protein, 4 aa, Artificial Sequence
  endomorphin-2 derivative
  MOD_RES (1)..(1): modified by Le
  MOD_RES (4)..(4): modified by NHNHAc
YPFF

SEQ ID NO: 233 - protein, 4 aa, Artificial Sequence
  endomorphin-2 derivative
  MOD_RES (1)..(1): modified by Ln
  MOD_RES (4)..(4): modified by NHNHAc
YPFF

SEQ ID NO: 234 - protein, 4 aa, Artificial Sequence
  endomorphin-2 derivative
  MOD_RES (1)..(1): modified by Na
  MOD_RES (4)..(4): modified by NHNHAc
YPFF

SEQ ID NO: 235 - protein, 4 aa, Artificial Sequence
  endomorphin-2 derivative
  MOD_RES (1)..(1): modified by Np
  MOD_RES (4)..(4): modified by NHNHAc
YPFF

SEQ ID NO: 236 - protein, 4 aa, Artificial Sequence
  endomorphin-2 derivative
  MOD_RES (1)..(1): modified by Oa
  MOD_RES (4)..(4): modified by NHNHAc
YPFF

SEQ ID NO: 237 - protein, 4 aa, Artificial Sequence
  endomorphin-2 derivative
  MOD_RES (1)..(1): modified by Pe
  MOD_RES (4)..(4): modified by NHNHAc
YPFF

SEQ ID NO: 238 - protein, 4 aa, Artificial Sequence
  endomorphin-2 derivative
  MOD_RES (4)..(4): carboxy terminus COR2 is CN
YPFF

SEQ ID NO: 239 - protein, 4 aa, Artificial Sequence
  endomorphin-2 derivative
  MOD_RES (1)..(1): modified by Ab
  MOD_RES (4)..(4): carboxy terminus COR2 is CN
YPFF

SEQ ID NO: 240 - protein, 4 aa, Artificial Sequence
  endomorphin-2 derivative
  MOD_RES (1)..(1): modified by Ac
  MOD_RES (4)..(4): carboxy terminus COR2 is CN
YPFF

SEQ ID NO: 241 - protein, 4 aa, Artificial Sequence
  endomorphin-2 derivative
  MOD_RES (1)..(1): modified by Ba
  MOD_RES (4)..(4): carboxy terminus COR2 is CN
YPFF

SEQ ID NO: 242 - protein, 4 aa, Artificial Sequence
  endomorphin-2 derivative
  MOD_RES (1)..(1): modified by Bo
  MOD_RES (4)..(4): carboxy terminus COR2 is CN
YPFF

SEQ ID NO: 243 - protein, 4 aa, Artificial Sequence
  endomorphin-2 derivative
  MOD_RES (1)..(1): modified by Bz
  MOD_RES (4)..(4): carboxy terminus COR2 is CN
YPFF

SEQ ID NO: 244 - protein, 4 aa, Artificial Sequence
  endomorphin-2 derivative
  MOD_RES (1)..(1): modified by Fo
  MOD_RES (4)..(4): carboxy terminus COR2 is CN
YPFF

SEQ ID NO: 245 - protein, 4 aa, Artificial Sequence
  endomorphin-2 derivative
  MOD_RES (1)..(1): modified by Hd
  MOD_RES (4)..(4): carboxy terminus COR2 is CN
YPFF

SEQ ID NO: 246 - protein, 4 aa, Artificial Sequence
  endomorphin-2 derivative
  MOD_RES (1)..(1): modified by He
  MOD_RES (4)..(4): carboxy terminus COR2 is CN
YPFF

SEQ ID NO: 247 - protein, 4 aa, Artificial Sequence
  endomorphin-2 derivative
  MOD_RES (1)..(1): modified by Hp
  MOD_RES (4)..(4): carboxy terminus COR2 is CN
YPFF

SEQ ID NO: 248 - protein, 4 aa, Artificial Sequence
  endomorphin-2 derivative
  MOD_RES (1)..(1): modified by Ip
  MOD_RES (4)..(4): carboxy terminus COR2 is CN
YPFF

SEQ ID NO: 249 - protein, 4 aa, Artificial Sequence
  endomorphin-2 derivative
  MOD_RES (1)..(1): modified by Le
  MOD_RES (4)..(4): carboxy terminus COR2 is CN
YPFF

SEQ ID NO: 250 - protein, 4 aa, Artificial Sequence
  endomorphin-2 derivative
  MOD_RES (1)..(1): modified by Ln
  MOD_RES (4)..(4): carboxy terminus COR2 is CN
YPFF

SEQ ID NO: 251 - protein, 4 aa, Artificial Sequence
  endomorphin-2 derivative
  MOD_RES (1)..(1): modified by Na
  MOD_RES (4)..(4): carboxy terminus COR2 is CN
YPFF

SEQ ID NO: 252 - protein, 4 aa, Artificial Sequence
  endomorphin-2 derivative
  MOD_RES (1)..(1): modified by Np
  MOD_RES (4)..(4): carboxy terminus COR2 is CN
YPFF

SEQ ID NO: 253 - protein, 4 aa, Artificial Sequence
  endomorphin-2 derivative
  MOD_RES (1)..(1): modified by Oa
  MOD_RES (4)..(4): carboxy terminus COR2 is CN
YPFF

SEQ ID NO: 254 - protein, 4 aa, Artificial Sequence
  endomorphin-2 derivative
  MOD_RES (1)..(1): modified by Pa
  MOD_RES (4)..(4): carboxy terminus COR2 is CN
YPFF

SEQ ID NO: 255 - protein, 4 aa, Artificial Sequence
  endomorphin-2 derivative
  MOD_RES (1)..(1): modified by Pc
  MOD_RES (4)..(4): carboxy terminus COR2 is CN
YPFF

SEQ ID NO: 256 - protein, 4 aa, Artificial Sequence
  endomorphin-2 derivative
  MOD_RES (1)..(1): modified by Pe
  MOD_RES (4)..(4): carboxy terminus COR2 is CN
YPFF

SEQ ID NO: 257 - protein, 4 aa, Artificial Sequence
  endomorphin-2 derivative
  MOD_RES (1)..(1): modified by Pg
  MOD_RES (4)..(4): carboxy terminus COR2 is CN
YPFF

SEQ ID NO: 258 - protein, 4 aa, Artificial Sequence
  endomorphin-2 derivative
  MOD_RES (1)..(1): modified by Ba
  MOD_RES (4)..(4): modified by NHCH3
YPFF

SEQ ID NO: 259 - protein, 4 aa, Artificial Sequence
  endomorphin-2 derivative
  MOD_RES (1)..(1): modified by He
  MOD_RES (4)..(4): modified by NHCH3
YPFF

SEQ ID NO: 260 - protein, 4 aa, Artificial Sequence
  endomorphin-2 derivative
  MOD_RES (1)..(1): modified by Hp
  MOD_RES (4)..(4): modified by NHCH3
YPFF

SEQ ID NO: 261 - protein, 4 aa, Artificial Sequence
  endomorphin-2 derivative
  MOD_RES (1)..(1): modified by Ip
  MOD_RES (4)..(4): modified by NHCH3
YPFF

SEQ ID NO: 262 - protein, 4 aa, Artificial Sequence
  endomorphin-2 derivative
  MOD_RES (1)..(1): modified by Le
  MOD_RES (4)..(4): modified by NHCH3
YPFF

SEQ ID NO: 263 - protein, 4 aa, Artificial Sequence
  endomorphin-2 derivative
  MOD_RES (1)..(1): modified by Ln
  MOD_RES (4)..(4): modified by NHCH3
YPFF

SEQ ID NO: 264 - protein, 4 aa, Artificial Sequence
  endomorphin-2 derivative
  MOD_RES (1)..(1): modified by Na
  MOD_RES (4)..(4): modified by NHCH3
YPFF

SEQ ID NO: 265 - protein, 4 aa, Artificial Sequence
  endomorphin-2 derivative
  MOD_RES (1)..(1): modified by Np
  MOD_RES (4)..(4): modified by NHCH3
YPFF

SEQ ID NO: 266 - protein, 4 aa, Artificial Sequence
  endomorphin-2 derivative
  MOD_RES (1)..(1): modified by Oa
  MOD_RES (4)..(4): modified by NHCH3
YPFF

SEQ ID NO: 267 - protein, 4 aa, Artificial Sequence
  endomorphin-2 derivative
  MOD_RES (1)..(1): modified by Pe
  MOD_RES (4)..(4): modified by NHCH3
YPFF

SEQ ID NO: 268 - protein, 4 aa, Artificial Sequence
  endomorphin-2 derivative
  MOD_RES (1)..(1): modified by Ip
  MOD_RES (4)..(4): modified by NHOH
YPFF

SEQ ID NO: 269 - protein, 4 aa, Artificial Sequence
  endomorphin-2 derivative
  MOD_RES (1)..(1): modified by Le
  MOD_RES (4)..(4): modified by NHOH
YPFF

SEQ ID NO: 270 - protein, 4 aa, Artificial Sequence
  endomorphin-2 derivative
  MOD_RES (1)..(1): modified by Ln
  MOD_RES (4)..(4): modified by NHOH
YPFF

SEQ ID NO: 271 - protein, 4 aa, Artificial Sequence
  endomorphin-2 derivative
  MOD_RES (1)..(1): modified by Np
  MOD_RES (4)..(4): modified by NHOH
YPFF

SEQ ID NO: 272 - protein, 4 aa, Artificial Sequence
  endomorphin-1 derivative
  MOD_RES (1)..(1): modified by Ac
  MOD_RES (4)..(4): modified by NH2
YPFF

SEQ ID NO: 273 - protein, 4 aa, Artificial Sequence
  endomorphin-1 derivative
  MOD_RES (1)..(1): modified by Pa
  MOD_RES (4)..(4): modified by NH2
YPFF

SEQ ID NO: 274 - protein, 4 aa, Artificial Sequence
  endomorphin-1 derivative
  MOD_RES (1)..(1): modified by Pg
  MOD_RES (4)..(4): modified by NH2
YPFF

SEQ ID NO: 275 - protein, 4 aa, Artificial Sequence
  endomorphin-2 derivative
  MOD_RES (1)..(1): modified by Ac
  MOD_RES (4)..(4): modified by OEt
YPFF

SEQ ID NO: 276 - protein, 4 aa, Artificial Sequence
  endomorphin-1 derivative
YPWF

SEQ ID NO: 277 - protein, 4 aa, Artificial Sequence
  endomorphin-2 derivative
YPFF

SEQ ID NO: 278 - protein, 7 aa, Artificial Sequence
  opioid peptide
YPFPGPI

SEQ ID NO: 279 - protein, 4 aa, Artificial Sequence
  opioid peptide
YPWT

SEQ ID NO: 280 - protein, 4 aa, Artificial Sequence
  opioid peptide
  MOD_RES (4)..(4): modified by NH2
YPLG

SEQ ID NO: 281 - protein, 4 aa, Artificial Sequence
  opioid peptide
  MOD_RES (4)..(4): modified by NH2
YPWG

(19) United States
(12) Patent Application Publication
(10) Pub. No.: US 2019/0248834 A1
(43) Pub. Date: Aug. 15, 2019

(54) ENDOMORPHIN-2, TETRAPEPTIDE DERIVATIVES THEREOF, AND USES THEREOF

(71) Applicants: Ruey J. YU, Chalfont, PA (US); Eugene J. VAN SCOTT, Abington, PA (US)

(72) Inventors: Ruey J. YU, Chalfont, PA (US); Eugene J. VAN SCOTT, Abington, PA (US)

(21) Appl. No.: 16/313,192

(22) PCT Filed: Jun. 26, 2017
(86) PCT No.: PCT/US17/39218

§ 371 Date: Dec. 26, 2018

Related U.S. Application Data
(60) Provisional application No. 62/428,283, filed on Nov. 30, 2016.
(60) Provisional application No. 62/354,962, filed on Jun. 27, 2016.

(51) Int. Cl. C07K 5/107; A61P 19/02

(52) U.S. Cl. CPC C07K 5/1016; A61K 38/00; A61P 19/02

(57) ABSTRACT

Endomorphin-2 and tetrapeptide derivatives thereof are described. Pharmaceutical or cosmetic compositions containing endomorphin-2 or a tetrapeptide derivative thereof are therapeutically effective to treat, improve or prevent pain in human subjects, such as pain associated with a disorder, disease, condition, symptom, or syndrome of the nervous system, musculoskeletal system, vascular system, immune system, tumors or cancers, including, but not limited to, pain associated with arthritis, headache, muscles or joints upon topical or systemic administration.

### CROSS-REFERENCE TO RELATED APPLICATIONS

This application claims priority under 35 U.S.C. §119 to U.S. Provisional Patent Application No. 62/354,962, filed on Jun. 27, 2016, and U.S. Provisional Patent Application No. 62/428,283, filed on Nov. 30, 2016, and the disclosures of which are herein incorporated by reference in their entireties.

### FIELD OF THE INVENTION

The invention relates to novel compounds, compositions and uses of the compositions comprising an endomorphin-2 or related tetrapeptide derivative having an amino acid sequence Tyr-Pro-Phe-Phe (SEQ ID NO: 1) for topical or systemic administration to alleviate or improve pain associated with a disease, disorder, condition, symptom or syndrome of the nervous system, musculoskeletal system, immune system, vascular system, tumors or cancers in human subjects, such as arthritis, joint pain, muscle pain, and headaches.

### BACKGROUND OF THE INVENTION

Endomorphins are endogenous tetrapeptide derivatives having analgesic effects. The endomorphins include endomorphin-2 (Tyr-Pro-Phe-Phe-NH₂; SEQ ID NO:1) and endomorphin-1 (Tyr-Pro-Trp-Phe-NH₂; SEQ ID NO: 2).

In 1997, Zadina's group synthesized a number of tetrapeptides named endomorphin-1 and endomorphin-2, which were discovered and identified in the bovine brain and human cortex, and showed remarkable affinity for the μ-opioid receptor. See, e.g., Zadina J E et al., “A potent and selective endogenous agonist for the mu-opiate receptor,” Nature (1997) 386: 499-502; Zadina JE., et al., “Cyclic analogues of Tyr-W-MIF-1 with prolonged analgesic activity and potency comparable to DAMGO and morphine,” Peptides (1994) 15: 1567-1569; and Zadina J E, et al., “Endomorphins: novel endogenous μ-opiate receptor agonists in regions of high μ-opiate receptor density,” Ann. NY Acad. Sci. (1999) 897: 136-144 (1999).

Goldberg I E et al., “Pharmacological characterization of endomorphin-1 and endomorphin-2 in mouse brain,” J. Pharmacol. Exp. Ther. (1998) 286(2): 1007-13 reported that both endomorphin-1 and endomorphin-2 did not have any affinity for either delta or kappa receptors, but very high affinity for mu receptors.

Horvath G, “Endomorphin-1 and endomorphin-2: pharmacology of the selective endogenous μ-opioid receptor agonists,” Pharmacol. Ther. (2000) 88(3): 437-63 reported that endomorphin-1 and endomorphin-2 appear to have properties consistent with neurotransmitter/neuromodulator actions in humans.

Chu X P et al., “Endomorphin-1 and endomorphin-2, endogenous ligands for the μ-opioid receptor, inhibit electrical activity of rat rostral ventrolateral medulla neurons in vitro,” Neuroscience (1999) 93(2): 681-6 reported that endomorphin-1 and endomorphin-2 significantly inhibited spontaneous discharge of the medullary neurons. Endomorphin-1 was found to be a more potent inhibitor than endomorphin-2.

Przewlocki et al. “Pain inhibition by endomorphins,” Ann. NY Acad. Sci. (1999) 897: 154-64, reported a study showing that rats injected with endomorphin-2 at 5-10 μg exhibited analgesic effects.

Mehta et al. “Endomorphin-1: Induction of Motor Behavior and Lack of Receptor Desensitization,” The Journal of Neuroscience (2001), 21(12): 4436-4442, reported a study showing that rats injected with 18 pmol of endomorphin-1 did not induce rapid desensitization of the motor response. This result showed that endomorphin-1 did not impair motor action that would be caused by morphine.

Fichna, J. et al., “The Endomorphin System and Its Evolving Neurophysiological Role,” Pharmacological Reviews (2007) 59(1): 88-123 reported that endomorphin-1 and endomorphin-2 are two endogenous opioid peptides with high affinity and selectivity for the μ-opioid receptor. The neuroanatomical distribution of endomorphins reflects their potential endogenous role in many major physiological processes, which include perception of pain, responses related to stress, and complex functions such as reward, arousal, and vigilance, as well as autonomic, cognitive, neuroendocrine, and limbic homeostasis.

The endomorphins have been found in the brain and stored in neurons and axon terminals. The most outstanding effect of the endomorphins is their anti-nociceptive action. This depends on both central and peripheral neurons. Additionally, the endomorphins cause vasodilation by stimulating nitric oxide release from the endothelium.

There are three major opioid receptors, namely mu (μ), delta (δ), and kappa (κ), which are found in the central and peripheral nervous systems that mediate the biological functions of opioids. After the discovery of the δ-opioid receptor-selective enkephalins in 1975, naturally occurring opioid peptides shown to bind preferentially to the μ-opioid receptor were identified including β-casomorphin (Tyr-Pro-Phe-Pro-Gly-Pro-Ile; SEQ ID NO: 278) from the tryptic digests of (3-casein, hemorphin-4 (Tyr-Pro-Trp-Thr; SEQ ID NO: 279) from digests of hemoglobin, and Tyr-Pro-Leu-Gly-NH₂ (SEQ ID NO: 280) and Tyr-Pro-Trp-Gly-NH₂ (SEQ ID NO: 281), both isolated from the brain. However, until 1997, no human peptide was identified that would show substantial μ₂-opioid receptor affinity and selectivity.

The μ receptor was later discovered to have two subtypes namely, μ₁ and μ₂. Studies showed that μ₂-opioid receptors are stimulated by both endomorphin-1 and endomorphin-2, whereas μ₁-opioid receptors are stimulated only by endomorphin-2. Further studies revealed that μ₁-opioid receptors mediate supraspinal analgesia and modulate acetylcholine (ACh) and prolactin release, whereas μ₂-opioid receptors mediate spinal analgesia, respiratory depression, and inhibition of gastrointestinal transit.

International Patent Application Publication WO 95/22557 to Brown et al., entitled “Novel Opioid Peptides for the Treatment of Pain and Use Thereof” (published 24 Aug. 1995) describes more than 51 various peptides, including endomorphin-2, as opioid receptor peptides. The opioid activity of the peptides was assessed in vitro using the guinea pig ileum longitudinal muscle preparation. The analgesic activity was determined by writhing models and hot plate test in rodents via intraperitoneal injection or subcutaneous injection. As shown in Table 1 (Page 27) of WO 95/22557, endomorphin-2 (#756) had an ED₅₀>20, which indicates that it is not active as an analgesic substance as compared to the more preferred non-endomorphin compounds (see Page 17 line 24-29 of WO 95/22557). The 50 other disclosed peptides are unrelated to endomorphin-2, and the active compounds are #1774, #2462, #2463, #2687 and #2690 as shown in Table 1 on pages 27-30 of WO 95/22557, which are unrelated to endomorphins.

International Patent Application Publication WO 98/42732 to Zadina et al. entitled “Mu-Opiate Receptor Peptides” (published 1 Oct. 1998) describes about 30 various peptides as specific for μ-opioid receptors. Among these peptides are endomorphin-1 and endomorphin-2. The in vivo or in vitro bioactivity of the peptide was examined in tests after intracerebroventricular or intrathecal injection to mice or guinea pigs. None of the tests were carried out by topical application, and no tests were carried out in human subjects. There is no description about compositions or use of compositions for analgesic effects in human subjects. The preferred tetrapeptides and derivatives disclosed in WO 98/42732 include endomorphin-1, H-Tyr-Pro-Trp-Phe-OH (SEQ ID NO: 276), and H-Tyr-Pro-Phe-Phe-OH (SEQ ID NO: 277).

International Patent Application Publication WO 03/020304 A2 to Jessop, D. et al., entitled “Inflammation Modulatory Compound” (published 13 Mar. 2003), describes the use of endomorphins in the treatment or prophylaxis of inflammation. Adjuvant arthritis was artificially induced in rats by intradermal injection (0.1 ml) of heat-killed M butyricum (Mycobacterium butyricum) in paraffin oil (10 mg/ml). After the onset of inflammation, endomorphin-1 (1 μmol in saline) was administered by intraperitoneal injection on days 9, 10, 11, 12 and 13 after adjuvant injection. The anti-inflammatory effect was measured by observing a decrease in rat paw volume on day 14. As shown in Examples 2, 3 and 4 at pages 5-8 of WO 03/020304 endomorphin-1 decreases rat paw volume by about 16% and 20% as compared to the control saline. Animal studies were also carried out to measure the production of endomorphins in organs and tissues including immune cells and synovial tissues of rats after adjuvant arthritis induction. In contrast to endomorphin-1, which was found to increase in the spleen and thymus of arthritic rats, endomorphin-2 was found to be the same as that of control group. This reference made some assumptions that anti-inflammatory effects can be determined by a reduction of edema or retention of fluid in rat paw, and made the further assumption that anti-inflammatory action is anti-arthritic.

The studies described above were all performed in animals (non-humans) by systemic injection. There is no description in any of the above references of the use of endomorphin-2 or derivatives thereof in a cosmetic or pharmaceutical compositions for topical or systemic administration to treat pain caused by medical disorders or symptoms in humans.

U.S. Pat. No. 8,101,574 B2 to Gillon, V. et al., entitled “Oligopeptides and Compositions Containing the Oligopeptides” (patented 24 Jan. 2012) describes a cosmetic composition containing endomorphin-1 or endomorphin-2, and the N-acetyl derivatives to reduce skin irritation by cosmetic ingredients. The irritant cosmetic ingredients include vitamin A and alpha-hydroxyacid. The concentration of N-acetyl-endomorphin-1 used in Example 1 of U.S. Pat. No. 8,101,574 was 0.0003% by weight (see column 19); the concentration of endomorphin-2 used in Example 2 was 0.001% by weight (see column 19); and the concentration of N-acetyl-endomorphin-2 used in Example 3 was 0.001% by weight (column 20). U.S. Pat. No. 8,101,574 discloses a maximum concentration of 0.1% by weight of endomorphin-2 and derivatives thereof (see, e.g., claims 2 and 5 at column 26). There is no disclosure of the use of endomorphin-2 or its derivatives in a cosmetic composition for topical or systemic administration to treat pain caused by medical disorders or symptoms. See, also U.S. Pat. No. 8,399,415 B2 to Gillon, V. et al. entitled “Oligopeptides and Cosmetic Compositions Containing the Oligopeptides” (patented 19 Mar. 2013), which is a continuation application of U.S. Pat. No. 8,101,574 and contains substantially the same disclosure.

International PCT Patent Application Publication No. WO2013/103634 A2 (filed Jan. 3, 2013; published Jul. 11, 2013) to Yu et al. entitled “N-Acylpeptide Derivatives and Their Uses” describes various peptide derivatives, including tripeptide derivatives up to eicosapeptide (C20) derivatives.

However, there is no description in WO2013/103634 of endomorphin-2 peptides or derivatives thereof.

Zadina, J E et al., “Endomorphin analog analgesics with reduced abuse liability, respiratory depression, motor impairment, tolerance, and glial activation relative to morphine,” Neuropharmacology (June 2016) Vol. 105; 215-227 (published online Dec. 31, 2015) discloses that opioids acting on the μ-opioid receptor are the most effective analgesics. However, adverse side effects severely limit their use. Of particular importance, abuse liability results in major medical, societal, and economic problems. For example, respiratory depression is the cause of fatal overdoses, and tolerance complicates treatment and increases the risk of side effects. Motor and cognitive impairment are especially problematic for older adults. Despite the host of negative side effects, opioids such as morphine are commonly used for acute and chronic pain conditions. In the disclosed study, researchers injected rats with morphine or endomorphins, and confirmed that the motor skills and breathing of the rats were significantly impaired in those receiving morphine. In contrast, rats receiving endomorphins experienced no substantial respiratory depression or impairment of motor skills. The pain relief offered by the endomorphins was equal to or greater than the morphine.

Grosser T., et al. “Time for nonaddictive relief of pain,” Science (Mar. 10, 2017), 1026-1027 explains that much has been written recently about the prevalence of chronic pain, the dramatic increase in opioid prescriptions in the United States over the past 15 years, the concomitant rise in opioid dependency and addiction, and the quadrupling of deaths from opioid abuse. Although indispensable for managing acute severe traumatic pain and pain in a palliative setting, most opioids are prescribed either by dentists or by primary practitioners for chronic nonmalignant pain, and marketed aggressively to consumers for the latter, despite no scientific evidence supporting such treatment beyond 12 weeks. On the contrary, chronic opioid use can itself lead to pain. Most abuse (perhaps 70%) involves access to opioids that are prescribed for others a diversion problem.

The references regarding endomorphin-2 described above can be summarized as folbows:
(1) A Cosmetic composition or use of the cosmetic composition containing preferably a low concentration, such as 0.001% by weight, of endomorphin-2 or its N-acetyl derivative was described for use in reducing irritation caused by cosmetic agents including vitamin A and alpha-hydroxyacids; the maximum concentration of endomorphin-2 or its N-acetyl derivative disclosed for use in a such a cosmetic composition was 0.1% by weight (see, e.g., U.S. Pat. Nos. 8,1101,574; 8,399,415); and
(2) The anti-inflammatory effects of endomorphin-2 among other non-endomorphin related compounds administered by intraperitoneal injection to rats was measured by observing a reduced edema or fluid in a rat paw assay; endomorphin-1 was shown to reduce edema by 16-20%, whereas endomorphin-2 did not have any effect that significantly differed from the effect of the control group (see, e.g., WO 03/020304).

To the best of the knowledge of the inventors, there is no disclosure in any of the above described references of the topical administration of endomorphin-2 or derivatives thereof for analgesic effects in human subjects.

### BRIEF SUMMARY OF THE INVENTION

It is now discovered that endomorphin-2 and derivatives thereof are therapeutically effective for alleviating or improving pain associated with a disease, disorder, condition, symptom or syndrome related to the nervous system, immune system, musculoskeletal system, vascular system, tumors or cancers, such as arthritis, when administered to a human subject, particularly a disease, disorder, condition, symptom or syndrome of the nervous system or musculoskeletal system, such as arthritis, joint pain, muscle pain, and headaches.

More specifically, the inventors discovered that endomorphin-2 and derivatives thereof are therapeutically effective as analgesic substances for treating pain associated with arthritis, migraine headache, acute common headache, osteoarthritis, psoriatic arthritis, and various other pains in human subjects, particularly when topically administered. The inventors also discovered that endomorphin-2 derivatives are therapeutically effective for systemic administration to alleviate or improve pain, such as pain associated with osteoarthritis in human subjects.

In one general aspect, the invention relates to an endomorphin-2 tetrapeptide derivative of formula (I):

R₁-Tyr-Pro-Phe-Phe-R₂ formula (I)

or a pharmaceutically acceptable salt thereof, wherein R₁ is H or an acyl radical having up to 29 carbon atoms; R₂ is NHR₃ or NHNHR₄, or alternatively the carboxy terminus —COR₂ is CN; R₃ is H, OH, an alkyl, aralkyl or aryl radical having up to 9 carbon atoms; and R₄ is H, an alkyl, aralkyl, aryl or acyl radical having up to 9 carbon atoms.

In another general aspect, the invention relates to a composition for topical administration comprising at least 0.2% by weight or volume of endomorphin-2 or a tetrapeptide derivative thereof of formula (I):

R₁-Tyr-Pro-Phe-Phe-R₂ formula (I)

or a pharmaceutically acceptable salt thereof, and optionally a pharmaceutically or cosmetically acceptable carrier,
wherein R₁ is H or an acyl radical having up to 29 carbon atoms; R₂ is NHR₃ or NHNHR₄, or alternatively the carboxy terminus —COR₂ is CN; R₃ is H, OH, an alkyl, aralkyl or aryl radical having up to 9 carbon atoms; and R₄ is H, an alkyl, aralkyl, aryl or acyl radical having up to 9 carbon atoms.

In yet another general aspect, the invention relates to a method for treating pain in a human subject in need thereof, the method comprising administering to the human subject a composition comprising a therapeutically effective amount of endomorphin-2 or a tetrapeptide derivative thereof of formula (I):

R₁-Tyr-Pro-Phe-Phe-R₂ formula (I)

or a pharmaceutically acceptable salt thereof, and optionally a pharmaceutically or cosmetically acceptable carrier, wherein R₁ is an acyl radical having up to 29 carbon atoms; R₂ is NHR₃ or NHNHR₄, or alternatively the carboxy terminus —COR₂ is CN; R₃ is H, OH, an alkyl, aralkyl or aryl radical having up to 9 carbon atoms; and R₄ is H, an alkyl, aralkyl, aryl or acyl radical having up to 9 carbon atoms.

In one embodiment, the composition is administered topically. In another embodiment, the composition is administered systemically.

In particular embodiments of the invention, the pain is associated with a disease, disorder, condition, symptom or syndrome selected from the group consisting of arthritis (e.g., osteoarthritis, psoriatic arthritis, etc.), headache (e.g., migraine headache, hangover headache, etc.), dental pain, lipoma, muscle pain, pharyngitis, sprain, trauma, sunburn, thermal burn, viral infection, herpes zoster, wounds, post-operative sites and injection sites.

Other aspects, features and advantages of the invention will be apparent from the following disclosure, including the detailed description of the invention and its preferred embodiments, and the appended claims.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood by one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Common or certain knowledge, scientific and medical terminologies can be readily found via interne, textbooks of chemistry, biochemistry, medicinal chemistry, pharmacology, dermatology and general medicine. The following are some examples. Robert K. Murray et al. eds. “Harper's Illustrated Biochemistry” 26^{th} edn. Vol. I-II, McGraw Hill, 2003. Laurence L. Brunton et al. eds. “Goodman & Gilman's The Pharmacological Basis of Therapeutics” 12^{th} edn. McGraw Hill Medical, 2011. Klaus Wolff et al. eds. “Fitzpatrick's Dermatology in General Medicine” 7^{th} edn. Vol. I-II, McGraw Hill Medical, New York, 2008. Tony Burns et al. eds. “Rook's Textbook of Dermatology” 8^{th} edn. Vol. I-IV, Wiley-Blackwell, 2010. Anthony S. Fauci et al. eds. “Harrison's Principles of Internal Medicine” 17^{th} edn, McGraw Hill Medical, New York, 2008.

An amino acid is an organic acid having one or more than one alkaline radicals such as amino, guanidino, imino, or hydrazine radical attached at any carbon atom other than carbon one. There are 20 common amino acids which are represented by chemical names, such as “glycine”, or abbreviated symbols such as three letters, “Gly” or one letter “G. In this disclosure, both one letter and three letters will be used. Except glycine, all other common amino acids have stereoisomers, i.e., enantiomer, D or L form. The amino acids in most natural peptides and proteins are all in L-form. Some D-form amino acids are produced by microorganism or present in antibiotics, and have inhibitory or antagonistic actions. For example, D-alanine, D-aspartic acid, and D-glutamic acid are present in bacterial cell walls, and D-glutamic acid, D-aspartic acid and D-phenylalanine are present in antibiotic bacitracin. An uncommon amino acid is an amino acid that is not a common amino acid. Examples of uncommon amino acids include, but are not limited to, β-alanine and taurine. The uncommon amino acids can exist as a D or L form.

The one letter and three letter symbols used for the 20 common amino acids are as follows: alanine (A, Ala), arginine (R, Arg), aspartic acid (D, Asp), asparagine (N, Asn), cysteine (C, Cys), glycine (G, Gly), glutamic acid (E, Glu), glutamine (Q, Gln), histidine (H, His), isoleucine (I, Ile), leucine (L, Leu), lysine (K, Lys), methionine (M, Met), phenylalanine (F, Phe), proline (P, Pro), serine (S, Ser), threonine (T, Thr), tryptophan (W, Trp), tyrosine (Y, Tyr) and valine (V, Val). The letter “O” or “Non” represents there is no amino acid.

A peptide bond, C(═O)NH, is a covalent bond formed between two amino acid molecules when the carboxyl group on one amino acid reacts with the amino group of the other amino acid in a dehydration synthesis reaction. A tetrapeptide contains 4 amino acid residues.

The terms and abbreviations that can be used are as follows: Ab, 2-acetoxybenzoyl; Ac, acetyl; Ba, butanoyl; Bo, benzyloxycarbonyl; Bz, benzoyl; Fo, formyl; Hd, hexadecanoyl; He, hexanoyl; Hp, heptanoyl; Ip, 2-(4-isobutylphenyl)propanoyl or Ibuprofen radical,; Le, linoleic; Ln, linolenic; Na, nonanoyl; Np, 2-(6-methoxy-2-naphthyl)propanoyl or Naproxen radical; Oa, octanoyl; Pa, propanoyl; Pc, phenylacetyl; Pe, pentanoyl; Pg, pyroglutamyl; ethyl ester, OEt; propyl ester, OPr; methyl ester, OMe.

As used herein, “endomorphin-2” refers to the tetrapeptide Tyr-Pro-Phe-Phe-NH₂(SEQ ID NO: 1). As used herein “endomorphin-1” refers to the tetrapeptide Tyr-Pro-Trp-Phe-NH₂ (SEQ ID NO: 2).

The term “derivative” when used with reference to a peptide, such as a tetrapeptide, refers to a peptide having a substitution or modification at the N-terminus and/or C-terminus of the peptide. An “endomorphin-2 derivative” in particular refers to the tetrapeptide endomorphin-2 having a substitution or modification at the N-terminus and/or C-terminus. Exemplary N-terminal modifications include, but are not limited to Ab, Ac, Ba, Bo, Bz, Fo, Hd, He, Hp, Ip, Le, Ln, Na, Np, Oa, Pa, Pc, Pe and Pg. Exemplary C-terminal modifications include, but are not limited to, NHCH₃, NHCH₂CH₃, NHCH₂CH₂CH₃; NHCH(CH₃)₂; NHC₆H₅; NHCH₂C₆H₅; NHNH₂; NHNHCH₃; NHNHCH₂CH₃; NHNHCH₂CH₂CH₃; NHNHCH(CH₃)₂; NHNHC₆H₅; NHNHCH₂C₆H₅; NHNHAc; NHNHPa; NHNHBz; NHNHOa; NHNHFo; and NHOH.

As used herein, the term “subject” means any animal, preferably a mammal, most preferably a human, to who will be or has been administered compounds (e.g., endomorphin-2 or a tetrapeptide derivative thereof) or compositions according to embodiments of the invention. The term “mammal” as used herein, encompasses any mammal. Examples of mammals include, but are not limited to, cows, horses, sheep, pigs, cats, dogs, mice, rats, rabbits, guinea pigs, monkeys, humans etc., more preferably, a human. In preferred embodiments of the invention, a subject is a human subject.

In one embodiment, “treatment” or “treating” refers to amelioration, improvement, prophylaxis, or reversal of a disease or disorder, or at least one discernible symptom thereof. In another embodiment, “treatment” or “treating” refers to amelioration, improvement, prophylaxis, or reversal of at least one measurable physical parameter related to the disease or disorder being treated, not necessarily discernible in or by the mammal or subject. In yet another embodiment, “treatment” or “treating” refers to inhibiting or slowing the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

In certain embodiments, compounds of interest are administered as a preventative measure. As used herein, “prevention” or “preventing” refers to a reduction of the risk of acquiring a given disease or disorder.

As used herein, a “therapeutically effective amount” of endomorphin-2 or a tetrapeptide derivative thereof of an embodiment of the invention means the amount of the endomorphin-2 or tetrapeptide derivative thereof that elicits the biological or medicinal response in a tissue system, animal or human that is being sought by a researcher, veterinarian, medical doctor or other clinician, which includes alleviation of the symptoms of the disease, condition, syndrome or disorder being treated. One skilled in the art will recognize that the therapeutically effective amount of the endomorphin-2 or tetrapeptide derivative thereof to be used in the invention can vary with factors, such as the particular subject, e.g., age, diet, health, etc., severity and complications and types of the symptom or disorder sought to be treated or prevented, the formulation used, etc.

One general aspect of the invention relates to endomorphin-2 or a tetrapeptide derivative thereof of formula (I):

R₁-Tyr-Pro-Phe-Phe-R₂ formula (I)

or a pharmaceutically acceptable salt thereof, wherein R₁ is H or an acyl radical having up to 29 carbon atoms; R₂ is NHR₃ or NHNHR₄, or alternatively the carboxy terminus —COR₂ is CN; R₃ is H, OH, an alkyl, aralkyl or aryl radical having up to 9 carbon atoms; and R₄ is H, an alkyl, aralkyl, aryl or acyl radical having up to 9 carbon atoms.

A typical acyl radical suitable for use in the invention includes, but is not limited to, Ab (2-acetoxybenzoyl), Ac (acetyl), Ba (butanoyl), Bo (benzyloxycarbonyl), Bz (benzoyl), Fo (formyl), Hd (hexadecanoyl), He (hexanoyl), Hp (heptanoyl), Ip (2-(4-isobutylphenyl)propanoyl; or ibuprofen radical), Le (linoleic), Ln (linolenic), Na (nonanoyl), Np (2-(6-methoxy-2-naphthyl)propanoyl; or Naproxen radical), Oa (octanoyl), Pa (propanoyl), Pc (phenylacetyl), Pe (pentanoyl), and Pg (pyroglutamyl).

In some embodiments, endomorphin-2 tetrapeptide derivatives of the invention have a nitrile (CN) group at the C-terminus. In such embodiments, the carboxy terminus of the tetrapeptide derivative of formula (I), i.e., —COR₂, is —CN, which is a dehydrated (minus H₂O) form of an amide group (—CONH₂). For example, the endomorphin-2 tetrapeptide derivative Tyr-Pro-Phe-Phe-CN (SEQ ID NO: 238) has a nitrile —CN group in place of —COR₂ at the C-terminus.

In particular embodiments of the endomorphin-2 tetrapeptide derivative of formula (I) of the invention, when R₂ is NH₂, then R₁ is not acetyl (Ac) or H.

Based on formula (I), illustrative endomorphin-2 and tetrapeptide derivatives thereof of the invention include, but are not limited to, the following:

Endomorphin-2 is the tetrapeptide derivative Tyr-Pro-Phe-Phe-NH₂ (SEQ ID NO: 1).

Representative endomorphin-2 tetrapeptide derivatives of formula (I) according to the invention, wherein R₁ is H, include, but are not limited to: Tyr-Pro-Phe-Phe-NHCH₃; Tyr-Pro-Phe-Phe-NHCH₂CH₃; Tyr-Pro-Phe-Phe-NHCH₂CH₂CH₃; Tyr-Pro-Phe-Phe-NHCH(CH₃)₂; Tyr-Pro-Phe-Phe-NHC₆H₅; Tyr-Pro-Phe-Phe-NHCH₂C₆H₅; Tyr-Pro-Phe-Phe-NHCH₂CH₂C₆H₅; Tyr-Pro-Phe-Phe-NHNH₂; Tyr-Pro-Phe-Phe-NHNHCH₃; Tyr-Pro-Phe-Phe-NHNHCH₂CH₃; Tyr-Pro-Phe-Phe-NHNHCH₂CH₂CH₃; Tyr-Pro-Phe-Phe-NHNHCH(CH₃)₂; Tyr-Pro-Phe-Phe-NHNHC₆H₅; Tyr-Pro-Phe-Phe-NHNHCH₂C₆H₅; Tyr-Pro-Phe-Phe-NHNHAc; Tyr-Pro-Phe-Phe-NHNHPa; Tyr-Pro-Phe-Phe-NHNHBz; Tyr-Pro-Phe-Phe-NHNHOa; Tyr-Pro-Phe-Phe-NHNHFo; and Tyr-Pro-Phe-Phe-NHOH (SEQ ID NOs: 3-22, respectively).

Representative endomorphin-2 tetrapeptide derivatives of formula (I) according to the invention, wherein R₁ is acetyl (Ac), include, but are not limited to: N-Ac-Tyr-Pro-Phe-Phe-NH₂; N-Ac-Tyr-Pro-Phe-Phe-NHCH₃; N-Ac-Tyr-Pro-Phe-Phe-NHCH₂CH₃; N-Ac-Tyr-Pro-Phe-Phe-NHCH₂CH₂CH₃; N-Ac-Tyr-Pro-Phe-Phe-NHCH(CH₃)₂; N-Ac-Tyr-Pro-Phe-Phe-NHC₆H₅; N-Ac-Tyr-Pro-Phe-Phe-NHCH₂C₆H₅; N-Ac-Tyr-Pro-Phe-Phe-NHCH₂CH₂C₆H₅; N-Ac-Tyr-Pro-Phe-Phe-NHNH₂; N-Ac-Tyr-Pro-Phe-Phe-NHNHCH₃; N-Ac-Tyr-Pro-Phe-Phe-NHNHCH₂CH₃; N-Ac-Tyr-Pro-Phe-Phe-NHNHCH₂CH₂CH₃; N-Ac-Tyr-Pro-Phe-Phe-NHNHCH(CH₃)₂; N-Ac-Tyr-Pro-Phe-Phe-NHNHC₆H₅; N-Ac-Tyr-Pro-Phe-Phe-NHNHCH₂C₆H₅; N-Ac-Tyr-Pro-Phe-Phe-NHNHAc; N-Ac-Tyr-Pro-Phe-Phe-NHNHPa; N-Ac-Tyr-Pro-Phe-Phe-NHNHBz; N-Ac-Tyr-Pro-Phe-Phe-NHNHOa; N-Ac-Tyr-Pro-Phe-Phe-NHNHFo; and N-Ac-Tyr-Pro-Phe-Phe-NHOH (SEQ ID NOs: 23-43, respectively).

Representative endomorphin-2 tetrapeptide derivatives of formula (I) according to the invention, wherein R₁ is pyroglutamyl (Pg), include, but are not limited to: N-Pg-Tyr-Pro-Phe-Phe-NH₂; N-Pg-Tyr-Pro-Phe-Phe-NHCH₃; N-Pg-Tyr-Pro-Phe-Phe-NHCH₂CH₃; N-Pa-Tyr-Pro-Phe-Phe-NHCH₂CH₂CH₃; N-Pg-Tyr-Pro-Phe-Phe-NHCH(CH₃)₂; N-Pa-Tyr-Pro-Phe-Phe-NHC₆H₅; N-Pg-Tyr-Pro-Phe-Phe-NHCH₂C₆H₅; N-Pg-Tyr-Pro-Phe-Phe-NHNH_{2;} N-Pg-Tyr-Pro-Phe-Phe-NHNHCH₃; N-Pg-Tyr-Pro-Phe-Phe-NHNHCH₂CH₃; N-Pg-Tyr-Pro-Phe-Phe-NHNHCH₂CH₂CH₃; N-Pg-Tyr-Pro-Phe-Phe-NHNHCH(CH₃)₂; N-Pg-Tyr-Pro-Phe-Phe-NHNHC₆H₅; N-Pa-Tyr-Pro-Phe-Phe-NHNHCH₂C₆H₅; N-Pg-Tyr-Pro-Phe-Phe-NHNHAc; N-Pg-Tyr-Pro-Phe-Phe-NHNHPa; N-Pg-Tyr-Pro-Phe-Phe-NHNHBz; N-Pg-Tyr-Pro-Phe-Phe-NHNHOa; N-Pg-Tyr-Pro-Phe-Phe-NHNHFo; and N-Pg-Tyr-Pro-Phe-Phe-NHOH (SEQ ID NOs: 44-63, respectively).

Representative endomorphin-2 tetrapeptide derivatives of formula (I) according to the invention, wherein R₁ is propanoyl (Pa), include, but are not limited to: N-Pa-Tyr-Pro-Phe-Phe-NH₂; N-Pa-Tyr-Pro-Phe-Phe-NHCH₃; N-Pa-Tyr-Pro-Phe-Phe-NHCH₂CH₃; N-Pa-Tyr-Pro-Phe-Phe-NHCH₂CH₂CH₃; N-Pa-Tyr-Pro-Phe-Phe-NHCH(CH₃)₂; N-Pa-Tyr-Pro-Phe-Phe-NHC₆H₅; N-Pa-Tyr-Pro-Phe-Phe-NHCH₂C₆H₅; N-Pa-Tyr-Pro-Phe-Phe-NHNH₂; N-Pa-Tyr-Pro-Phe-Phe-NHNHCH₃; N-Pa-Tyr-Pro-Phe-Phe-NHNHCH₂CH₃; N-Pa-Tyr-Pro-Phe-Phe-NHNHCH₂CH₂CH₃; N-Pa-Tyr-Pro-Phe-Phe-NHNHCH(CH₃)₂; N-Pa-Tyr-Pro-Phe-Phe-NHNHC₆H₅; N-Pa-Tyr-Pro-Phe-Phe-NHNHCH₂C₆H₅; N-Pa-Tyr-Pro-Phe-Phe-NHNHAc; N-Pa-Tyr-Pro-Phe-Phe-NHNHPa; N-Pa-Tyr-Pro-Phe-Phe-NHNHBz; N-Pa-Tyr-Pro-Phe-Phe-NHNHOa; N-Pa-Tyr-Pro-Phe-Phe-NHNHFo; and N-Pa-Tyr-Pro-Phe-Phe-NHOH (SEQ ID NOs: 64-83, respectively).

Representative endomorphin-2 tetrapeptide derivatives of formula (I) according to the invention, wherein R₁ is benzoyl (Bz), include, but are not limited to: N-Bz-Tyr-Pro-Phe-Phe-NH_{2;} N-Bz-Tyr-Pro-Phe-Phe-NHCH₃; N-Bz-Tyr-Pro-Phe-Phe-NHCH₂CH₃; N-Bz-Tyr-Pro-Phe-Phe-NHCH₂CH₂CH₃; N-Bz-Tyr-Pro-Phe-Phe-NHCH(CH₃)₂; N-Bz-Tyr-Pro-Phe-Phe-NHC₆H₅; N-Bz-Tyr-Pro-Phe-Phe-NHCH₂C₆H₅; N-Bz-Tyr-Pro-Phe-Phe-NHNH₂; N-Bz-Tyr-Pro-Phe-Phe-NHNHCH₃; N-Bz-Tyr-Pro-Phe-Phe-NHNHCH₂CH₃; N-Bz-Tyr-Pro-Phe-Phe-NHNHCH₂CH₂CH₃; N-Bz-Tyr-Pro-Phe-Phe-NHNHCH(CH₃)₂; N-Bz-Tyr-Pro-Phe-Phe-NHNHC₆H₅; N-Bz-Tyr-Pro-Phe-Phe-NHNHCH₂C₆H₅; N-Bz-Tyr-Pro-Phe-Phe-NHNHAc; N-Bz-Tyr-Pro-Phe-Phe-NHNHPa; N-Bz-Tyr-Pro-Phe-Phe-NHNHBz; N-Bz-Tyr-Pro-Phe-Phe-NHNHOa; N-Bz-Tyr-Pro-Phe-Phe-NHNHFo; and N-Bz-Tyr-Pro-Phe-Phe-NHOH (SEQ ID NOs: 84-103, respectively).

Representative endomorphin-2 tetrapeptide derivatives of formula (I) according to the invention, wherein R₁ is Formyl (Fo), include, but are not limited to: N-Fo-Tyr-Pro-Phe-Phe-NH₂; N-Fo-Tyr-Pro-Phe-Phe-NHCH₃; N-Fo-Tyr-Pro-Phe-Phe-NHCH₂CH₃; N-Fo-Tyr-Pro-Phe-Phe-NHCH₂CH₂CH₃; N-Fo-Tyr-Pro-Phe-Phe-NHCH(CH₃)₂; N-Fo-Tyr-Pro-Phe-Phe-NHC₆H₅; N-Fo-Tyr-Pro-Phe-Phe-NHCH₂C₆H₅; N-Fo-Tyr-Pro-Phe-Phe-NHNH₂; N-Fo-Tyr-Pro-Phe-Phe-NHNHCH₃; N-Fo-Tyr-Pro-Phe-Phe-NHNHCH₂CH₃; N-Fo-Tyr-Pro-Phe-Phe-NHNHCH₂CH₂CH₃; N-Fo-Tyr-Pro-Phe-Phe-NHNHCH(CH₃)₂; N-Fo-Tyr-Pro-Phe-Phe-NHNHC₆H₅; N-Fo-Tyr-Pro-Phe-Phe-NHNHCH₂C₆H₅; N-Fo-Tyr-Pro-Phe-Phe-NHNHAc; N-Fo-Tyr-Pro-Phe-Phe-NHNHPa; N-Fo-Tyr-Pro-Phe-Phe-NHNHBz; N-Fo-Tyr-Pro-Phe-Phe-NHNHOa; N-Fo-Tyr-Pro-Phe-Phe-NHNHFo; and N-Fo-Tyr-Pro-Phe-Phe-NHOH (SEQ ID NOs: 104-123, respectively).

Representative endomorphin-2 tetrapeptide derivatives of formula (I) according to the invention, wherein R₁ is 2-acetoxybenzoyl, (Ab), include, but are not limited to: N-Ab-Tyr-Pro-Phe-Phe-NH₂; N-Ab-Tyr-Pro-Phe-Phe-NHCH₃; N-Ab-Tyr-Pro-Phe-Phe-NHCH₂CH₃; N-Ab-Tyr-Pro-Phe-Phe-NHCH₂CH₂CH₃; N-Ab-Tyr-Pro-Phe-Phe-NHCH(CH₃)₂; N-Ab-Tyr-Pro-Phe-Phe-NHC₆H₅; N-Ab-Tyr-Pro-Phe-Phe-NHCH₂C₆H₅; N-Ab-Tyr-Pro-Phe-Phe-NHNH₂; N-Ab-Tyr-Pro-Phe-Phe-NHNHCH₃; N-Ab-Tyr-Pro-Phe-Phe-NHNHCH₂CH₃; N-Ab-Tyr-Pro-Phe-Phe-NHNHCH₂CH₂CH₃; N-Ab-Tyr-Pro-Phe-Phe-NHNHCH(CH₃)₂; N-Ab-Tyr-Pro-Phe-Phe-NHNHC₆H₅; N-Ab-Tyr-Pro-Phe-Phe-NHNHCH₂C₆H₅; N-Ab-Tyr-Pro-Phe-Phe-NHNHAc; N-Ab-Tyr-Pro-Phe-Phe-NHNHPa; N-Ab-Tyr-Pro-Phe-Phe-NHNHBz; N-Ab-Tyr-Pro-Phe-Phe-NHNHOa; N-Ab-Tyr-Pro-Phe-Phe-NHNHFo; and N-Ab-Tyr-Pro-Phe-Phe-NHOH (SEQ ID NOs: 124-143, respectively).

Representative endomorphin-2 tetrapeptide derivatives of formula (I) according to the invention, wherein R₁ is benzyloxycarbonyl (Bo), include, but are not limited to: N-Bo-Tyr-Pro-Phe-Phe-NH₂; N-Bo-Tyr-Pro-Phe-Phe-NHCH₃; N-Bo-Tyr-Pro-Phe-Phe-NHCH₂CH₃; N-Bo-Tyr-Pro-Phe-Phe-NHCH₂CH₂CH₃; N-Bo-Tyr-Pro-Phe-Phe-NHCH(CH₃)₂; N-Bo-Tyr-Pro-Phe-Phe-NHC₆H₅; N-Bo-Tyr-Pro-Phe-Phe-NHCH₂C₆H₅; N-Bo-Tyr-Pro-Phe-Phe-NHNH₂; N-Bo-Tyr-Pro-Phe-Phe-NHNHCH₃; N-Bo-Tyr-Pro-Phe-Phe-NHNHCH₂CH₃; N-Bo-Tyr-Pro-Phe-Phe-NHNHCH₂CH₂CH₃; N-Bo-Tyr-Pro-Phe-Phe-NHNHCH(CH₃)₂; N-Bo-Tyr-Pro-Phe-Phe-NHNHC₆H₅; N-Bo-Tyr-Pro-Phe-Phe-NHNHCH₂C₆H₅; N-Bo-Tyr-Pro-Phe-Phe-NHNHAc; N-Bo-Tyr-Pro-Phe-Phe-NHNHPa; N-Bo-Tyr-Pro-Phe-Phe-NHNHBz; N-Bo-Tyr-Pro-Phe-Phe-NHNHOa; N-Bo-Tyr-Pro-Phe-Phe-NHNHFo; and N-Bo-Tyr-Pro-Phe-Phe-NHOH (SEQ ID NOs: 144-163, respectively).

Representative endomorphin-2 tetrapeptide derivatives of formula (I) according to the invention, wherein R₁ is hexadecanoyl (Hd), include, but are not limited to: N-Hd-Tyr-Pro-Phe-Phe-NH₂; N-Hd-Tyr-Pro-Phe-Phe-NHCH₃; N-Hd-Tyr-Pro-Phe-Phe-NHCH₂CH₃; N-Hd-Tyr-Pro-Phe-Phe-NHCH₂CH₂CH₃; N-Hd-Tyr-Pro-Phe-Phe-NHCH(CH₃)₂; N-Hd-Tyr-Pro-Phe-Phe-NHC₆H₅; N-Hd-Tyr-Pro-Phe-Phe-NHCH₂C₆H₅; N-Hd-Tyr-Pro-Phe-Phe-NHCH₂CH₂C₆H₅; N-Hd-Tyr-Pro-Phe-Phe-NHNH₂; N-Hd-Tyr-Pro-Phe-Phe-NHNHCH₃; N-Hd-Tyr-Pro-Phe-Phe-NHNHCH₂CH₃; N-Hd-Tyr-Pro-Phe-Phe-NHNHCH₂CH₂CH₃; N-Hd-Tyr-Pro-Phe-Phe-NHNHCH(CH₃)₂; N-Hd-Tyr-Pro-Phe-Phe-NHNHC₆H₅; N-Hd-Tyr-Pro-Phe-Phe-NHNHCH₂C₆H₅; N-Hd-Tyr-Pro-Phe-Phe-NHNHAc; N-Hd-Tyr-Pro-Phe-Phe-NHNHPa; N-Hd-Tyr-Pro-Phe-Phe-NHNHBz; and N-Hd-Tyr-Pro-Phe-Phe-NHOH (SEQ ID NOs: 164-182, respectively).

Representative endomorphin-2 tetrapeptide derivatives of formula (I) according to the invention, wherein R₁ is phenylacetyl (Pc), include, but are not limited to: N-Pc-Tyr-Pro-Phe-Phe-NH₂; N-Pc-Tyr-Pro-Phe-Phe-NHCH₃; N-Pc-Tyr-Pro-Phe-Phe-NHCH₂CH₃; N-Pc-Tyr-Pro-Phe-Phe-NHCH₂CH₂CH₃; N-Pc-Tyr-Pro-Phe-Phe-NHCH(CH₃)₂; N-Pc-Tyr-Pro-Phe-Phe-NHC₆H₅; N-Pc-Tyr-Pro-Phe-Phe-NHCH₂C₆H₅; N-Pc-Tyr-Pro-Phe-Phe-NHCH₂CH₂C₆H_{5;} N-Pc-Tyr-Pro-Phe-Phe-NHNH₂; N-Pc-Tyr-Pro-Phe-Phe-NHNHCH₃; N-Pc-Tyr-Pro-Phe-Phe-NHNHCH₂CH₃; N-Pc-Tyr-Pro-Phe-Phe-NHNHCH₂CH₂CH₃; N-Pc-Tyr-Pro-Phe-Phe-NHNHCH(CH₃)₂; N-Pc-Tyr-Pro-Phe-Phe-NHNHC₆H₅; N-Pc-Tyr-Pro-Phe-Phe-NHNHCH₂C₆H₅; N-Pc-Tyr-Pro-Phe-Phe-NHNHAc; N-Pc-Tyr-Pro-Phe-Phe-NHNHPa; N-Pc-Tyr-Pro-Phe-Phe-NHNHBz; and N-Pc-Tyr-Pro-Phe-Phe-NHOH (SEQ ID NOs: 183-201, respectively).

Representative endomorphin-2 tetrapeptide derivatives of formula (I) according to the invention, wherein R₁ is butanoyl (Ba); pentanoyl (Pe); hexanoyl (He); heptanoyl (Hp); octanoyl (Oa); or nonanoyl (Na), include, but are not limited to: N-Ba-Tyr-Pro-Phe-Phe-NH₂; N-Pe-Tyr-Pro-Phe-Phe-NH₂; N-He-Tyr-Pro-Phe-Phe-NH₂; N-Hp-Tyr-Pro-Phe-Phe-NH₂; N-Oa-Tyr-Pro-Phe-Phe-NH₂; N-Na-Tyr-Pro-Phe-Phe-NH₂; N-Ba-Tyr-Pro-Phe-Phe-NHNH₂; N-Pe-Tyr-Pro-Phe-Phe-NHNH₂; N-He-Tyr-Pro-Phe-Phe-NHNH_{2;} N-Hp-Tyr-Pro-Phe-Phe-NHNH₂; N-Oa-Tyr-Pro-Phe-Phe-NHNH₂; N-Na-Tyr-Pro-Phe-Phe-NHNH₂; N-Ba-Tyr-Pro-Phe-Phe-NHOH; N-Pe-Tyr-Pro-Phe-Phe-NHOH; N-He-Tyr-Pro-Phe-Phe-NHOH; N-Hp-Tyr-Pro-Phe-Phe-NHOH; N-Oa-Tyr-Pro-Phe-Phe-NHOH; and N-Na-Tyr-Pro-Phe-Phe-NHOH (SEQ ID NOs: 202-219, respectively).

Preferred endomorphin-2 tetrapeptide derivatives, wherein R₂ is NH₂, NHNH₂ or NHNHAc, include, but are not limited to: N-Ab-Tyr-Pro-Phe-Phe-NH₂(SEQ ID NO: 124); N-Ac-Tyr-Pro-Phe-Phe-NH₂ (SEQ ID NO: 23); N-Ba-Tyr-Pro-Phe-Phe-NH₂ (SEQ ID NO: 202); N-Bo-Tyr-Pro-Phe-Phe-NH₂ (SEQ ID NO: 144); N-Bz-Tyr-Pro-Phe-Phe-NH₂ (SEQ ID NO: 84); N-Fo-Tyr-Pro-Phe-Phe-NH₂(SEQ ID NO: 104); N-Hd-Tyr-Pro-Phe-Phe-NH₂ (SEQ ID NO: 164); N-He-Tyr-Pro-Phe-Phe-NH₂ (SEQ ID NO: 204); N-Hp-Tyr-Pro-Phe-Phe-NH₂ (SEQ ID NO: 205); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Na-Tyr-Pro-Phe-Phe-NH₂ (SEQ ID NO: 207); N-Np-Tyr-Pro-Phe-Phe-NH₂ (SEQ ID NO: 223); N-Oa-Tyr-Pro-Phe-Phe-NH₂ (SEQ ID NO: 206); N-Pa-Tyr-Pro-Phe-Phe-NH₂ (SEQ ID NO: 64); N-Pc-Tyr-Pro-Phe-Phe-NH₂ (SEQ ID NO: 183); N-Pe-Tyr-Pro-Phe-Phe-NH₂ (SEQ ID NO: 203); N-Pg-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Ba-Tyr-Pro-Phe-Phe-NHNH₂ (SEQ ID NO: 208); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Fo-Tyr-Pro-Phe-Phe-NHNH₂ (SEQ ID NO: 111); N-Hd-Tyr-Pro-Phe-Phe-NHNH₂ (SEQ ID NO: 172); N-He-Tyr-Pro-Phe-Phe-NHNH₂ (SEQ ID NO: 210); N-Hp-Tyr-Pro-Phe-Phe-NHNH₂ (SEQ ID NO: 211); N-Ip-Tyr-Pro-Phe-Phe-NHNH₂ (SEQ ID NO: 225); N-Le-Tyr-Pro-Phe-Phe-NHNH₂ (SEQ ID NO: 226); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂ (SEQ ID NO: 218); N-Pa-Tyr-Pro-Phe-Phe-NHNH₂ (SEQ ID NO: 71); N-Pc-Tyr-Pro-Phe-Phe-NHNH₂ (SEQ ID NO: 191); N-Pe-Tyr-Pro-Phe-Phe-NHNH₂ (SEQ ID NO: 209); N-Pg-Tyr-Pro-Phe-Phe-NHNH₂(SEQ ID NO: 51); Tyr-Pro-Phe-Phe-NHNHAc (SEQ ID NO: 17); N-Ab-Tyr-Pro-Phe-Phe-NHNHAc (SEQ ID NO: 138); N-Ac-Tyr-Pro-Phe-Phe-NHNHAc (SEQ ID NO: 38); N-Ba-Tyr-Pro-Phe-Phe-NHNHAc (SEQ ID NO: 228); N-Bo-Tyr-Pro-Phe-Phe-NHNHAc (SEQ ID NO: 158); N-Bz-Tyr-Pro-Phe-Phe-NHNHAc (SEQ ID NO: 98); N-Fo-Tyr-Pro-Phe-Phe-NHNHAc (SEQ ID NO: 118); N-Hd-Tyr-Pro-Phe-Phe-NHNHAc (SEQ ID NO: 170); N-He-Tyr-Pro-Phe-Phe-NHNHAc (SEQ ID NO: 229); N-Hp-Tyr-Pro-Phe-Phe-NHNHAc (SEQ ID NO: 230); N-Ip-Tyr-Pro-Phe-Phe-NHNHAc (SEQ ID NO: 231); N-Le-Tyr-Pro-Phe-Phe-NHNHAc (SEQ ID NO: 232); N-Ln-Tyr-Pro-Phe-Phe-NHNHAc (SEQ ID NO: 233); N-Na-Tyr-Pro-Phe-Phe-NHNHAc (SEQ ID NO: 234); N-Np-Tyr-Pro-Phe-Phe-NHNHAc (SEQ ID NO: 235); N-Oa-Tyr-Pro-Phe-Phe-NHNHAc (SEQ ID NO: 236); N-Pa-Tyr-Pro-Phe-Phe-NHNHAc (SEQ ID NO: 78); N-Pc-Tyr-Pro-Phe-Phe-NHNHAc (SEQ ID NO: 198); N-Pe-Tyr-Pro-Phe-Phe-NHNHAc (SEQ ID NO: 237); and N-Pg-Tyr-Pro-Phe-Phe-NHNHAc (SEQ ID NO: 58).

Preferred endomorphin-2 tetrapeptide derivatives of formula (I) according to the invention, wherein the carboxyl end of Phe-R₂ is —CN, include, but are not limited to: Tyr-Pro-Phe-Phe-CN; N-Ab-Tyr-Pro-Phe-Phe-CN; N-Ac-Tyr-Pro-Phe-Phe-CN; N-Ba-Tyr-Pro-Phe-Phe-CN; N-Bo-Tyr-Pro-Phe-Phe-CN; N-Bz-Tyr-Pro-Phe-Phe-CN; N-Fo-Tyr-Pro-Phe-Phe-CN; N-Hd-Tyr-Pro-Phe-Phe-CN; N-He-Tyr-Pro-Phe-Phe-CN; N-Hp-Tyr-Pro-Phe-Phe-CN; N-Ip-Tyr-Pro-Phe-Phe-CN; N-Le-Tyr-Pro-Phe-Phe-CN; N-Ln-Tyr-Pro-Phe-Phe-CN; N-Na-Tyr-Pro-Phe-Phe-CN; N-Np-Tyr-Pro-Phe-Phe-CN; N-Oa-Tyr-Pro-Phe-Phe-CN; N-Pa-Tyr-Pro-Phe-Phe-CN; N-Pc-Tyr-Pro-Phe-Phe-CN; N-Pe-Tyr-Pro-Phe-Phe-CN; and N-Pg-Tyr-Pro-Phe-Phe-CN (SEQ ID NOs: 238-257, respectively).

Preferred endomorphin-2 tetrapeptide derivatives of formula (I) according to the invention, wherein R₂ is NHCH₃ or NHOH, include, but are not limited to: Tyr-Pro-Phe-Phe-NHCH₃ (SEQ ID NO: 3); N-Ab-Tyr-Pro-Phe-Phe- NHCH₃ (SEQ ID NO: 125); N-Ac-Tyr-Pro-Phe-Phe-NHCH₃ (SEQ ID NO: 24); N-Ba-Tyr-Pro-Phe-Phe-NHCH₃(SEQ ID NO: 258); N-Bo-Tyr-Pro-Phe-Phe-NHCH₃ (SEQ ID NO: 145); N-Bz-Tyr-Pro-Phe-Phe-NHCH₃ (SEQ ID NO: 125); N-Fo-Tyr-Pro-Phe-Phe-NHCH₃ (SEQ ID NO: 105); N-Hd-Tyr-Pro-Phe-Phe-NHCH₃(SEQ ID NO: 165); N-He-Tyr-Pro-Phe-Phe-NHCH₃ (SEQ ID NO: 259); N-Hp-Tyr-Pro-Phe-Phe-NHCH₃ (SEQ ID NO: 260); N-Ip-Tyr-Pro-Phe-Phe-NHCH₃NHCH₃(SEQ ID NO: 261); N-Le-Tyr-Pro-Phe-Phe-NHCH₃ (SEQ ID NO: 262); N-Ln-Tyr-Pro-Phe-Phe-NHCH₃(SEQ ID NO: 263); N-Na-Tyr-Pro-Phe-Phe-NHCH₃ (SEQ ID NO: 264); N-Np-Tyr-Pro-Phe-Phe-NHCH₃(SEQ ID NO: 265); N-Oa-Tyr-Pro-Phe-Phe-NHCH₃ (SEQ ID NO: 266); N-Pa-Tyr-Pro-Phe-Phe-NHCH₃(SEQ ID NO: 65); N-Pc-Tyr-Pro-Phe-Phe-NHCH₃ (SEQ ID NO: 184); N-Pe-Tyr-Pro-Phe-Phe-NHCH₃(SEQ ID NO: 267); N-Pa-Tyr-Pro-Phe-Phe-NHCH₃ (SEQ ID NO: 45); Tyr-Pro-Phe-Phe-NHOH (SEQ ID NO: 22); N-Ab-Tyr-Pro-Phe-Phe-NHOH (SEQ ID NO: 143); N-Ac-Tyr-Pro-Phe-Phe-NHOH (SEQ ID NO: 43); N-Ba-Tyr-Pro-Phe-Phe-NHOH (SEQ ID NO: 214); N-Bo-Tyr-Pro-Phe-Phe-NHOH (SEQ ID NO: 163); N-Bz-Tyr-Pro-Phe-Phe-NHOH (SEQ ID NO: 103); N-Fo-Tyr-Pro-Phe-Phe-NHOH (SEQ ID NO: 123); N-Hd-Tyr-Pro-Phe-Phe-NHOH (SEQ ID NO: 182); N-He-Tyr-Pro-Phe-Phe-NHOH (SEQ ID NO: 216); N-Hp-Tyr-Pro-Phe-Phe-NHOH (SEQ ID NO: 217); N-Ip-Tyr-Pro-Phe-Phe-NHOH (SEQ ID NO: 268); N-Le-Tyr-Pro-Phe-Phe-NHOH (SEQ ID NO: 269); N-Ln-Tyr-Pro-Phe-Phe-NHOH (SEQ ID NO: 270); N-Na-Tyr-Pro-Phe-Phe-NHOH (SEQ ID NO: 219); N-Np-Tyr-Pro-Phe-Phe-NHOH (SEQ ID NO: 271); N-Oa-Tyr-Pro-Phe-Phe-NHOH (SEQ ID NO: 218); N-Pa-Tyr-Pro-Phe-Phe-NHOH (SEQ ID NO: 83); N-Pc-Tyr-Pro-Phe-Phe-NHOH (SEQ ID NO: 201); N-Pe-Tyr-Pro-Phe-Phe-NHOH (SEQ ID NO: 215); and N-Pg-Tyr-Pro-Phe-Phe-NHOH (SEQ ID NO: 63).

The preferred endomorphin-2 and tetrapeptide derivatives thereof of formula (I) according to the invention include, but are not limited to: Tyr-Pro-Phe-Phe-NH₂ (SEQ ID NO: 1); N-Ab-Tyr-Pro-Phe-Phe-NH₂ (SEQ ID NO: 124); N-Ac-Tyr-Pro-Phe-Phe-NH₂ (SEQ ID NO: 23); N-Ba-Tyr-Pro-Phe-Phe-NH₂ (SEQ ID NO: 202); N-Bo-Tyr-Pro-Phe-Phe-NH₂ (SEQ ID NO: 144); N-Bz-Tyr-Pro-Phe-Phe-NH₂ (SEQ ID NO: 84); N-Fo-Tyr-Pro-Phe-Phe-NH₂ (SEQ ID NO: 104); N-Hd-Tyr-Pro-Phe-Phe-NH₂ (SEQ ID NO: 164); N-He-Tyr-Pro-Phe-Phe-NH₂ (SEQ ID NO: 204); N-Hp-Tyr-Pro-Phe-Phe-NH₂ (SEQ ID NO: 205); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Na-Tyr-Pro-Phe-Phe-NH₂ (SEQ ID NO: 207); N-Np-Tyr-Pro-Phe-Phe-NH₂ (SEQ ID NO: 223); N-Oa-Tyr-Pro-Phe-Phe-NH₂ (SEQ ID NO: 205); N-Pa-Tyr-Pro-Phe-Phe-NH₂ (SEQ ID NO: 64); N-Pc-Tyr-Pro-Phe-Phe-NH₂ (SEQ ID NO: 183); N-Pe-Tyr-Pro-Phe-Phe-NH₂ (SEQ ID NO: 203); N-Pa-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Ba-Tyr-Pro-Phe-Phe-NHNH₂ (SEQ ID NO: 208); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Fo-Tyr-Pro-Phe-Phe-NHNH₂ (SEQ ID NO: 111); N-Hd-Tyr-Pro-Phe-Phe-NHNH₂ (SEQ ID NO: 172); N-He-Tyr-Pro-Phe-Phe-NHNH₂ (SEQ ID NO: 210); N-Hp-Tyr-Pro-Phe-Phe-NHNH₂ (SEQ ID NO: 211); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂(SEQ ID NO: 212); N-Pa-Tyr-Pro-Phe-Phe-NHNH₂ (SEQ ID NO: 71); N-Pc-Tyr-Pro-Phe-Phe-NHNH₂ (SEQ ID NO: 191); N-Pe-Tyr-Pro-Phe-Phe-NHNH₂ (SEQ ID NO: 209); N-Pg-Tyr-Pro-Phe-Phe-NHNH₂ (SEQ ID NO: 51); Tyr-Pro-Phe-Phe-NHNHAc (SEQ ID NO: 17); N-Ab-Tyr-Pro-Phe-Phe-NHNHAc (SEQ ID NO: 138); N-Ac-Tyr-Pro-Phe-Phe-NHNHAc (SEQ ID NO: 38); N-Ba-Tyr-Pro-Phe-Phe-NHNHAc (SEQ ID NO: 228); N-Bo-Tyr-Pro-Phe-Phe-NHNHAc (SEQ ID NO: 158); N-Bz-Tyr-Pro-Phe-Phe-NHNHAc (SEQ ID NO: 98); N-Fo-Tyr-Pro-Phe-Phe-NHNHAc (SEQ ID NO: 118); N-Hd-Tyr-Pro-Phe-Phe-NHNHAc (SEQ ID NO: 179); N-He-Tyr-Pro-Phe-Phe-NHNHAc (SEQ ID NO: 229); N-Hp-Tyr-Pro-Phe-Phe-NHNHAc (SEQ ID NO: 230); N-Ip-Tyr-Pro-Phe-Phe-NHNHAc (SEQ ID NO: 231); N-Le-Tyr-Pro-Phe-Phe-NHNHAc (SEQ ID NO: 232); N-Ln-Tyr-Pro-Phe-Phe-NHNHAc (SEQ ID NO: 233); N-Na-Tyr-Pro-Phe-Phe-NHNHAc (SEQ ID NO: 234); N-Np-Tyr-Pro-Phe-Phe-NHNHAc (SEQ ID NO: 235); N-Oa-Tyr-Pro-Phe-Phe-NHNHAc (SEQ ID NO: 236); N-Pa-Tyr-Pro-Phe-Phe-NHNHAc (SEQ ID NO: 78); N-Pc-Tyr-Pro-Phe-Phe-NHNHAc (SEQ ID NO: 198); N-Pe-Tyr-Pro-Phe-Phe-NHNHAc (SEQ ID NO: 237); N-Pg-Tyr-Pro-Phe-Phe-NHNHAc (SEQ ID NO: 198); Tyr-Pro-Phe-Phe-CN (SEQ ID NO: 238); N-Ab-Tyr-Pro-Phe-Phe-CN (SEQ ID NO: 239); N-Ac-Tyr-Pro-Phe-Phe-CN (SEQ ID NO: 240); N-Ba-Tyr-Pro-Phe-Phe-CN (SEQ ID

NO: 241); N-Bo-Tyr-Pro-Phe-Phe-CN (SEQ ID NO: 242); N-Bz-Tyr-Pro-Phe-Phe-CN (SEQ ID NO: 243); N-Fo-Tyr-Pro-Phe-Phe-CN (SEQ ID NO: 244); N-Hd-Tyr-Pro-Phe-Phe-CN (SEQ ID NO: 245); N-He-Tyr-Pro-Phe-Phe-CN (SEQ ID NO: 246); N-Hp-Tyr-Pro-Phe-Phe-CN (SEQ ID NO: 247); N-Ip-Tyr-Pro-Phe-Phe-CN (SEQ ID NO: 248); N-Le-Tyr-Pro-Phe-Phe-CN (SEQ ID NO: 249); N-Ln-Tyr-Pro-Phe-Phe-CN (SEQ ID NO: 250); N-Na-Tyr-Pro-Phe-Phe-CN (SEQ ID NO: 251); N-Np-Tyr-Pro-Phe-Phe-CN (SEQ ID NO: 252); N-Oa-Tyr-Pro-Phe-Phe-CN (SEQ ID NO: 253); N-Pa-Tyr-Pro-Phe-Phe-CN (SEQ ID NO: 254); N-Pc-Tyr-Pro-Phe-Phe-CN (SEQ ID NO: 255); N-Pe-Tyr-Pro-Phe-Phe-CN (SEQ ID NO: 256); and N-Pg-Tyr-Pro-Phe-Phe-CN (SEQ ID NO: 257).

The more preferred endomorphin-2 tetrapeptide derivatives of formula (I) according to the invention include, but are not limited to: N-Ab-Tyr-Pro-Phe-Phe-NH₂ (SEQ ID NO: 124); N-Ac-Tyr-Pro-Phe-Phe-NH₂ (SEQ ID NO: 23); N-Ba-Tyr-Pro-Phe-Phe-NH₂ (SEQ ID NO: 202); N-Bo-Tyr-Pro-Phe-Phe-NH₂ (SEQ ID NO: 144); N-Bz-Tyr-Pro-Phe-Phe-NH₂ (SEQ ID NO: 84); N-Fo-Tyr-Pro-Phe-Phe-NH₂ (SEQ ID NO: 104); N-Hd-Tyr-Pro-Phe-Phe-NH₂ (SEQ ID NO: 164); N-He-Tyr-Pro-Phe-Phe-NH₂ (SEQ ID NO: 204); N-Hp-Tyr-Pro-Phe-Phe-NH₂ (SEQ ID NO: 205); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Na-Tyr-Pro-Phe-Phe-NH₂ (SEQ ID NO: 207); N-Np-Tyr-Pro-Phe-Phe-NH₂ (SEQ ID NO: 223); N-Oa-Tyr-Pro-Phe-Phe-NH₂ (SEQ ID NO: 205); N-Pa-Tyr-Pro-Phe-Phe-NH₂ (SEQ ID NO: 64); N-Pc-Tyr-Pro-Phe-Phe-NH₂ (SEQ ID NO: 183); N-Pe-Tyr-Pro-Phe-Phe-NH₂ (SEQ ID NO: 203); N-Pg-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Ba-Tyr-Pro-Phe-Phe-NHNH₂ (SEQ ID NO: 208); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Fo-Tyr-Pro-Phe-Phe-NHNH₂ (SEQ ID NO: 111); N-Hd-Tyr-Pro-Phe-Phe-NHNH₂ (SEQ ID NO: 172); N-He-Tyr-Pro-Phe-Phe-NHNH₂ (SEQ ID NO: 210); N-Hp-Tyr-Pro-Phe-Phe-NHNH₂ (SEQ ID NO: 211); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂ (SEQ ID NO: 212); N-Pa-Tyr-Pro-Phe-Phe-NHNH₂ (SEQ ID NO: 71); N-Pc-Tyr-Pro-Phe-Phe-NHNH₂ (SEQ ID NO: 191); N-Pe-Tyr-Pro-Phe-Phe-NHNH₂ (SEQ ID NO: 209); and N-Pg-Tyr-Pro-Phe-Phe-NHNH₂ (SEQ ID NO: 51).

The most preferred endomorphin-2 tetrapeptide derivatives of formula (I) of the invention include, but are not limited to: N-Ab-Tyr-Pro-Phe-Phe-NH₂(SEQ ID NO: 124); N-Ac-Tyr-Pro-Phe-Phe-NH₂ (SEQ ID NO: 23); N-Bo-Tyr-Pro-Phe-Phe-NH₂ (SEQ ID NO: 144); N-Bz-Tyr-Pro-Phe-Phe-NH₂ (SEQ ID NO: 84); N-Hd-Tyr-Pro-Phe-Phe-NH₂ (SEQ ID NO: 164); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂(SEQ ID NO: 222); N-Np-Tyr-Pro-Phe-Phe-NH₂ (SEQ ID NO: 223); N-Oa-Tyr-Pro-Phe-Phe-NH₂ (SEQ ID NO: 205); N-Pc-Tyr-Pro-Phe-Phe-NH₂ (SEQ ID NO: 183); N-Pa-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Hd-Tyr-Pro-Phe-Phe-NHNH₂ (SEQ ID NO: 172); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂(SEQ ID NO: 212); N-Pc-Tyr-Pro-Phe-Phe-NHNH₂ (SEQ ID NO: 191); and N-Pg-Tyr-Pro-Phe-Phe-NHNH₂ (SEQ ID NO: 51).

Endomorphin-2 and tetrapeptide derivatives thereof according to embodiments of the invention can be made or synthesized by any method known to those skilled in the art in view of the present disclosure. Methods of making peptides and peptide derivatives, such as by chemical synthesis, are well known to those of ordinary skill in the art in view of the present disclosure.

Chemical and physical properties, biological functions and analgesic effects of a peptide depend on the nature and sequence of amino acid residues, and different amino acid residues or different amino acid sequences may result in a completely different pharmacological actions. In addition to chemical and physical properties, biological functions and analgesic effects or potency of a peptide are also changed when the functional groups of such peptides are modified by substitution. In most cases, endomorphin-2 and the tetrapeptide derivatives thereof of the present invention have different and much improved chemical and physical properties, biological functions and analgesic effects as compared to unmodified tetrapeptides.

A peptide is usually an amphoteric substance, having a positively charged amino group and negatively charged carboxylic group in the same molecule. A peptide normally cannot penetrate the skin on topical application because of the tough stratum corneum layer acting as a permeation barrier. In general, an ionic substance, amphoteric substance or any substance with a molecular weight of more than 800 daltons usually cannot readily penetrate intact skin. The endomorphin-2 and tetrapeptide derivatives thereof of the invention typically have an amide or N-acyl form, so that they are no longer amphoteric in nature, and are readily bioavailable for penetration and/or distribution to target tissues or sites for pharmacological actions by topical administration.

Compositions and Methods of Use

Another general aspect of the invention relates to a composition comprising endomorphin-2 or a tetrapeptide derivative of formula (I) according to the invention, and optionally a pharmaceutically or cosmetically acceptable carrier. A composition according to the invention can comprise endomorphin-2 or any tetrapeptide derivative thereof of formula (I) described herein.

In typical embodiments of the invention, a composition comprises a therapeutically effective amount of endomorphin-2 or a tetrapeptide derivative of the invention. In view of the present disclosure, standard procedures can be performed to evaluate the effect of administration of a composition to a subject (e.g., determine whether a clinically observable beneficial effect is achieved), thus allowing a skilled artisan to determine the therapeutically effective amount of endomorphin-2 of a tetrapeptide derivative of the invention. A clinically observable beneficial effect can be a situation that, when a composition of the invention is administered to a subject after symptoms to be treated are observable, the symptoms are prevented from further development or aggravation, or develop to a lesser degree than without administration of the composition of the invention. The clinically observable beneficial effect can also be that, when a composition of the invention is administered to a subject before symptoms to be treated are observable, the symptoms are prevented from occurring or subsequently occur to a lesser degree than without administration of the composition.

In one embodiment, a therapeutically effective amount of endomorphin-2 or a tetrapeptide derivative thereof of the invention will alleviate a condition or discomfort associated with pain in a subject to be treated or who has been treated, for example, by at least about 20%, for example, by at least about 30%, by at least about 40%, by at least about 50%, by at least about 60%, by at least about 70%, by at least about 80%, by at least about 90%, or about 100%, preferably by at least about 25%; by at least about 50%, by at least about 75%, or by at least about 90% to 100%, relative to the condition or discomfort associated with pain prior to administration of a composition or endomorphin-2 or tetrapeptide derivative thereof of the invention.

In another embodiment, a therapeutically effective amount of endomorphin-2 or a tetrapeptide derivative thereof of the invention will reduce a disease, disorder, condition, symptom, or syndrome of the subject to be treated or who has been treated by. In another embodiment, a therapeutically effective amount of endomorphin-2 or a tetrapeptide derivative thereof of the invention will prevent a disease, disorder, condition, symptom, or syndrome of the subject to be treated, or reduce the probability of its onset, by at least about 20%, for example, by at least about 30%, by at least about 40%, by at least about 50%, by at least about 60%, by at least about 70%, by at least about 80%, by at least about 90%, or about 100%.

Compositions of the invention can be formulated for administration according to any method known in the art. Typically, the compositions are formulated for topical administration or systemic administration, and preferably are formulated for topical administration. The topical application includes administration to skin, eye, mucous membranes of the conjunctiva, nasopharynx, oropharynx, vagina, urethra, rectum, and anus. The systemic administration includes oral (enteral) administration and parenteral injections. The parenteral injections include intravenous injection or infusion, intra-arterial injection, subcutaneous injection, intramuscular injection, and intra-articular injection. Other routes of administration include sublingual administration, under the tongue, from oral mucosa bypassing the portal circulation, and pulmonary adsorption by inhaling and absorbing through the respiratory tract.

Compositions according to embodiments of the invention can further comprise a pharmaceutically or cosmetically acceptable carrier. Pharmaceutically and cosmetically acceptable carriers are well known to those of ordinary skill in the art and one of ordinary skill in the art would be able to select an appropriate pharmaceutical or cosmetically acceptable carrier for inclusion in a composition of the invention depending on a variety of factors including the type of composition, e.g., solution (aqueous or anhydrous), cream, etc., and intended route of administration, e.g., topical, systemic, etc., based on general knowledge in the art in view of the present disclosure.

In a preferred embodiment, a composition of the invention is formulated in any manner suitable for topical administration to a subject, preferably for topical application to skin of a subject For example, for topical application, a composition comprising endomorphin-2 or a tetrapeptide derivative thereof of formula (I) according to the invention can be formulated as a solution, gel, lotion, cream, oil-in-water emulsion, water-in-oil emulsion, ointment, shampoo, spray, stick, powder, mask, pads, mouth rinse or wash, vaginal gel or suppositories, rectal gel or suppositories, urethral gel or suppositories or other form acceptable for use on skin, nail, hair, oral mucosa, vaginal or anal mucosa, mouth or gums. The concentration of an active ingredient can be about 0.01% to about 99.9% by weight or volume (solution composition) of the total composition, with a preferred concentration of about 0.1% to about 30%, and with a more preferred concentration of about 0.2% to about 10% by weight or by volume (solution composition) of the total composition.

To prepare a topical composition, at least one tetrapeptide is dissolved in a solution prepared from water, ethanol, propylene glycol, butylene glycol, or other topically acceptable solvent. To prepare a topical composition in another form, a tetrapeptide can be incorporated as a fine powder form without dissolving, or alternatively first dissolving in water, ethanol, propylene glycol, or other solvent, and the solution thus obtained is mixed with a topically acceptable base or vehicle including a gel, lotion, cream, oil-in-water emulsion, water-in-oil emulsion, ointment, shampoo, spray, stick, powder, mask, pads, mouth rinse or wash, etc. Contemplated embodiments of the invention include concentration ranges of 0.001% to 0.01%, 0.01% to 0.1%, 0.1% to 0.2%, 0.2% to 0.3%, 0.3% to 0.4%, 0.4% to 0.5%, 0.5% to 0.6%, 0.6% to 0.7%, 0.7% to 0.8%, 0.8% to 0.9%, 0.9% to 1%, 1% to 2%, 2% to 3%, 3% to 4%, 4% to 5%, 5% to 6%, 6% to 7%, 7% to 8%, 8% to 9%, 9% to 10%, 10% to 14%, 14% to 18%, 18% to 22%, 22% to 26%, 26% to 30%, 30% to 35%, 35% to 40%, 40% to 45%, 45% to 50%, 50% to 60%, 60% to 70%, 70% to 80%, 80% to 90%, and 90% to 99.9% of a tetrapeptide, by weight or volume of the total composition.

In a particular embodiment of the invention, the composition is a composition for topical administration comprising at least 0.2% by weight or volume, based on a total weight or volume, of the composition, of endomorphin-2 or a tetrapeptide derivative thereof of formula (I) according to the invention. In certain embodiments, a topical composition of the invention comprises 0.2% to 10% by weight or volume, based on a total weight or volume of the composition, of endomorphin-2 or a tetrapeptide derivative thereof of formula (I) according to the invention, such as, for example 0.2%, 0.3%, 0.4%, 0.5%, 0.6%, 0.7%, 0.8%, 1%, 2%, 3%, 4%, 5%, or 10% by weight or volume.

In other embodiments, an endomorphin-2 or tetrapeptide derivative thereof of the invention can be formulated for oral administration, parenteral injections or other routes including oral mucosa, under the tongue administration, with or without pharmaceutically acceptable vehicle or carrier, to evaluate for systemic effects.

In oral preparations, endomorphin-2 or a tetrapeptide derivative thereof of the invention is formulated in powder, tablet form, gelatin capsules with or without mixing with gelatin powder, or in other form including a liquid or suspension form. Each tablet, capsule or unit dosage contains about 0.01 mg to about 100 mg, preferably about 0.1 mg to about 50 mg, and more preferably about 1 mg to about 25 mg of endomorphin-2 or a tetrapeptide derivative thereof. As an illustration, endomorphin-2 or a tetrapeptide derivative thereof in powder form, e.g. 1 mg, can be placed under the tongue without swallowing for a short time to achieve systemic administration. The daily dosage for a subject can vary, however in general is about 0.001 mg/kg to about 10 mg/kg, preferably about 0.01 mg to about 5 mg/kg, and more preferably about 0.1 mg to about 2 mg/kg body weight of the subject.

For parenteral injections, endomorphin-2 or a tetrapeptide derivative is prepared in a solution or suspension under sterilized conditions in concentration from about 0.01% to about 10%, preferably about 0.1% to about 5%, more preferably about 0.2% to about 2% weight by volume in water, propylene glycol, glycerol, polyethylene glycol, a mixture thereof, or in other vehicle or carrier. The other vehicle or carrier includes peanut oil, soybean oil, mineral oil, sesame oil, and the like. As an option, a thickener can be added into an injection composition to increase the viscosity, so that the composition has a comparable viscosity with the body fluid in the knee joints or other joints. As an illustration, but not limitation, the thickener can be selected from the group consisting of carboxymethylcellulose, sodium carboxymethylcellulose, casein, cellulose, gelatin, sodium hyaluronate, methylcellulose, PEG 200, PEG 300, PEG 400, PEG 600, PEG 3350, PEG 4000, polyglactin, polylactide, polypropylene glycol, polyvinyl alcohol, protamine sulfate, povidone, starch, captisol, dextran, dextrose, fructose, albumin, and lactose.

Another general aspect of the invention relates to a method for treating or preventing pain associated with a disease, disorder, condition, symptom, or syndrome associated with tumors, cancers, the nervous system, immune system, musculoskeletal system, vascular system, or other system in a subject in need thereof. The phrase “associated with,” as used herein with reference to pain and any particular disease, disorder, condition, symptom or syndrome, means that the pain is caused by the disease, disorder, condition, syndrome or symptom, or experienced, observed, or perceived by the subject at substantially the same time as the occurrence of the disease, disorder, condition, syndrome or symptom. For example, in one embodiment, “pain associated with arthritis” means that the pain in the subject is caused by the arthritis. In another embodiment, “pain associated with arthritis” means that the pain is experienced, observed, or perceived by the subject at substantially the same time as the occurrence of the arthritis in the subject.

According to embodiments of the invention, a method comprises administering to a subject a therapeutically effective amount of endomorphin-2 or a tetrapeptide derivative thereof according to the invention, or a composition comprising the same. Endomorphin-2, any endomorphin-2 tetrapeptide derivative, or any composition described herein is suitable for use in the methods of the invention.

According to embodiments of the invention, the composition can be administered alone or optionally in combination with another active ingredient. The composition and the other active ingredient can be administered simultaneously or sequentially to provide synergetic, synergistic, or enhancing effects. Examples of other active ingredients suitable for use in the methods of the invention include, but are not limited to, 2-acetoxybenzoic acid (Aspirin); 2-(4-isobutylphenyl)propanoic acid (Ibuprofen); and 2-(6-methoxy-2-naphthyl)propanoic acid (Naproxen).

Tetrapeptides and compositions of the invention can provide analgesic effects, and can thus be used to treat pain in a subject, preferably a human subject, including pain associated with a condition, disorder, disease, symptom or syndrome of (A) tumors and cancers, (B) the immune system, (C) the nervous system, (D) the vascular system, (E) the musculoskeletal system, and other tissues or systems, described as follows.

(A) Tumors and Cancers

Cancer is an unregulated proliferation of cells due to loss of normal controls, resulting in abnormal growth, lack of differentiation, local tissue invasion, and often, metastasis. A tumor is an abnormal growth of cells or tissues, which may be benign or malignant. Tumors or cancers that can be treated with a composition of the invention include, but are not limited to, pain associated with solid tumors or cancer in the body.

There are more than 50 different types of human cancers. Common cancers include, but are not limited to, bladder cancer, bone cancer, brain tumors, breast cancer, cervical cancer, colon cancer, colorectal cancer, esophageal cancer, head & neck cancers, kidney cancer, leukemias, liver cancer, lung cancer, lymphoma, melanoma, ovarian cancer, pancreatic cancer, prostate cancer, skin cancers, thyroid cancer, and uterine cancer.

(B) Immune System

The immune system, like organs such as the liver, kidney and thyroid, is composed of specialized cells that play a vital role in host defense. These cells include leukocytes (white blood cells) and dendritic cells. Deranged immune system can cause disorders including, but not limited to: (1) rheumatic, connective tissue or collagen diseases; (2) autoimmune diseases including rheumatoid arthritis, psoriasis and psoriatic arthritis; (3) liver diseases; (4) gastrointestinal diseases; (5) immune-mediated nephritis and vasculitis; (6) immune-mediated diseases of the nervous system and the eye; and (7) human immunodeficiency virus (HIV) and acquired immune deficiency syndrome (AIDS). Various pains can be caused by a deranged immune system.

(C) Nervous System

Pains directly associated with the nervous system that can be treated with the compositions of the invention including, but are not limited to, the following conditions or disorders, which may present as indicated, or otherwise: (1) dementia and Alzheimer's disease: progressive loss of memory, shrinkage and atrophy of cerebral cortex, tangles of fibers in nerve cells, senile plaques of β-amyloid, and/or decreased choline acetyltransferase enzyme; (2) carpal tunnel syndrome: weakness, pain, tingling, numbness, and/or burning in the palm and fingers; (3) encephalitis: inflammation of the brain; (4) headache: migraine, expansion of blood vessels pressing on nerves or constriction blocking blood supply, inflammation, and/or muscle contraction in the face, neck or scalp; (5) meningitis: infection of spinal fluid and meninges; (6) neuralgia: nerve pain, peripheral neuropathy, sciatica, shingles, and/or trigeminal neuralgia; (7) Parkinson's disease: tremors in limbs, and/or muscular rigidity; (8) amnesia: loss of memory and inability to form new memory; and (9) others, such as ataxia, Bell's palsy, epilepsy, multiple sclerosis, myasthenia gravis, narcolepsy, paralysis and/or rabies.

Alzheimer's disease causes progressive cognitive deterioration and is characterized by senile plaques of β-amyloid deposits, neurofibrillary tangles in the cerebral cortex and subcortical gray matter, and currently there is no cure.

Parkinson's disease is an idiopathic, slowly progressive, degenerative central nervous system (CNS) disorder characterized by resting tremor, muscular rigidity, slow and decreased movement, and postural instability, and currently there is no cure.

(D) Vascular System

The vascular conditions, reactions and disorders that can be treated with a composition of the present invention include, but are not limited to, sunburn, thermoburn, acanthosis nigricans, acrocyanosis, actinic cheilitis, actinic prurigo, dermatitis, dermatosis, dermographiacsm, dyshidrosis, drug eruptions, inflammation, eczema, erythema, erythema migrans, erythrocyanosis, erythromelalgia, familial hemorrhage, histamine reaction, inflammatory papular and pustular lesions, lichen planus, lupus erythematosus, mycosis fungoides, neurodermatitis, neuropeptide and neurovascular reactions, parapsoriasis, perniosis (chilblains), photoallergy, photoreaction, photosensitivity, pityriasis rosea, pityriasis rubra pilaris, polymorphic light eruption, psoriasis, rhinophyma, rosacea, sclerosis, spider naevi, T-cell disorders, telangiectasia, varicose veins (varicosis), urticaria, vessel dilation, and other vascular reactions.

(E) Musculoskeletal System

The conditions or abnormalities of the musculoskeletal system that can be treated with the compositions of the invention include, but are not limited to, the following conditions or disorders, which may present as indicated, or otherwise: (1) joint pain and muscle pain, arthritis, inflammation, swelling, pain, stiffness and decreased range of motion of joints, including neck, shoulder, elbow, wrist, lower back, hip, knee and ankle joints; (2) osteoporosis: reduction of calcium in bone leading to thin bone and bone susceptible to fracture; (3) osteoarthritis: inflammation of joint cartilage provoking swelling and pain; (3) rheumatoid arthritis: inflammation of synovium and destruction of cartilage, damage to heart, lungs, nerves and eyes; (5) ankylosing spondylitis: arthritis affecting sacroiliac joints and spine with inflammation and immovability; (6) bursitis: inflammation of bursa; (7) tendinitis: inflammation of tendon; (8) gout: recurrent acute arthritis from uric acid deposit; (9) psoriatic arthritis: inflammation, pain, stiffness, and swelling of joints; (10) muscle pain: soreness and achiness; (11) sprain (e.g., ankle, wrist or other joint): twisting of ligaments causing swelling and pain; and (12) specifically, join pain (e.g., neck, shoulder, elbow, wrist, lower back, hip, knee and ankle pains), inflammation, and arthritis.

The term “arthritis” generally refers to joint pain or joint disease, which can be acute or chronic. However, there are more than 100 types of arthritis, and the term “arthritis” also includes arthritis caused by diseases affecting systems other than the musculoskeletal system, such as the immune system, e.g., rheumatoid arthritis. For example, arthritis includes, but is not limited to, degenerative arthritis, inflammatory arthritis, infectious arthritis (e.g., arthritis caused by a bacterium, virus or fungus that can enter a joint and trigger inflammation), metabolic arthritis (e.g., high levels of metabolites, such as uric acid, leading to conditions, such as gout), rheumatoid arthritis, osteoarthritis, and psoriatic arthritis, some of which are described in greater detail above.

In certain embodiments of the invention, the pain is associated with a disease, disorder, condition, symptom, or syndrome of the nervous system or musculoskeletal system. Exemplary diseases, disorders, conditions, symptoms, or syndromes of the nervous system that can be treated according to embodiments of the invention include, but are not limited to, headache (e.g., migraine headache and hangover headache). Exemplary diseases, disorders, conditions, symptoms, or syndromes of the musculoskeletal system that can be treated according to embodiments of the invention include, but are not limited to, arthritis (e.g., osteoarthritis, psoriatic arthritis, etc.), muscle pain, and sprain.

Other exemplary diseases, disorders, conditions, symptoms or syndromes causing pain that can be treated with the tetrapeptides and compositions of the invention include, but are not limited to, dental pain, pharyngitis, lipoma, trauma, viral infection, sunburn, thermal burn, herpes zoster, and skin wounds (e.g., post-operative sites and injection sites).

In preferred embodiments of the invention, the pain is associated with arthritis, headache or muscle pain.

Treatment Dosages

Dosages and dosing frequency will be determined by a trained medical professional depending on the analgesic effectiveness of the endomorphin-2 or tetrapeptide derivative thereof that is used, the characteristics of the particular formulation, and the identity and severity of the disorder treated or prevented. One of ordinary skill in the art will be able to determine appropriate treatment dosage based on general knowledge in the art in view of the present disclosure.

Evaluation of Analgesic Effectiveness

In view of the present disclosure, standard procedures can be performed to evaluate the effect of the administration of a composition of the invention to a subject, thus allowing a skilled artisan to determine the therapeutically effective amount of the endomorphin-2 or tetrapeptide derivative thereof to be included in the composition, the route of administration, the dosing frequency. etc. Because pain is a subjective condition or symptom based on perception, sensation or reaction, the subject to be treated is preferably able to participate in the evaluation of the therapeutic efficacy of a composition of the invention.

For example, clinical evaluation of analgesic effectiveness can be defined as for example, 1+ (25%), which represents partial relief of pain for less than 4 hours; 2+ (50%), which represents substantial but incomplete relief of pain for less than 4 hours; 3+ (75%), which represents complete relief of pain for less than 4 hours; and 4+ (90-100%), which represents complete relief or eradication of pain for more than 4 hours. Such clinical evaluation can be used to determine the analgesic effectiveness of administration (e.g., topical administration) of a tetrapeptide derivative of the invention.

Based on the clinical findings described herein, topical or systemic administration of endomorphin-2 and the tetrapeptide derivatives thereof of the invention are therapeutically effective for treating pain, arthritis, inflammation, and other conditions, disorders, symptoms or syndromes associated with tumors, cancers, infections, the immune system, nervous system, musculoskeletal system, or other system.

More specifically, the inventors have made the following findings regarding the invention:
(1) Endomorphin-2 and derivatives thereof, but not endomorphin-1 and derivatives, are therapeutically effective as analgesic substances to eradicate pain upon topical administration to a human subject, and in certain instances the eradication of pain is near instantaneous upon administration. To the best of the knowledge of the inventors, analgesic effects in animals or humans upon topical application of endomorphin-2 and derivatives was neither taught nor described prior to the invention.
(2) Based on the clinical tests and studies described herein, endomorphin-2 tetrapeptide derivatives comprising an N-acyl group, such as N-acetyl, N-pyroglutamyl, or N-benzoyl and/or a C-terminal amide, hydroxylamine, or hydrazide group, such as N-alkyl amide and N-acyl hydrazide show the greatest analgesic effects.
(3) Endomorphin-2 derivatives are therapeutically effective in alleviating or improving pain, such as pain associated with osteoarthritis, in human subjects when systemically administered to the human subjects.
(4) Based on the studies conducted by the inventors as described herein, endomorphin-1 and derivatives thereof have minimal analgesic effects upon topical or systemic administration to human subjects. These findings were unexpected in view of published non-human animal studies reporting that systemic injection of endomorphin-1 in mice or rats had a significant analgesic effect.
(5) Based on the studies described herein, endomorphin-2 tetrapeptide derivatives comprising a C-terminal carboxyl group in the free acid or ester form had no detectable analgesic effect upon topical application to human subjects. Our findings are in contrast to that reported by WO98/42732.
(6) Based on the studies described herein, certain modifications, such as larger radical groups at the C-terminus (e.g., phenethyl amide), rendered the endomorphin-2 tetrapeptide derivative substantially ineffective as an analgesic substance upon topical application.
(7) Based on the studies described herein, certain modifications, such as larger radical groups at the N-terminus (e.g., N-benzoyl), rendered the endomorphin-2 tetrapeptide derivative more effective as an analgesic substance with more prolonged efficacious effects upon topical or systemic application in human subjects, as compared to the analgesic effects of tetrapeptides having smaller radical groups at the N-terminus (e.g., N-formyl).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the following Examples, test results and appended claims.

### EXAMPLES

In some of the following examples, the analgesic efficacy of topical or systemic administration of a tetrapeptide derivative of the invention in the treated subject was clinically evaluated according to the following scale:
1+ (25%): partial relief of pain for less than 4 hours;
2+ (50%): substantial but incomplete relief of pain for less than 4 hours
3+ (75%): complete relief of pain for less than 4 hours
4+ (90-100%): complete relief or eradication of pain for more than 4 hours.

Also in the following Examples, the description of the clinical results using phrases such as “pain disappeared completely” or “instantaneously” means that the pain was no longer perceived by the subject, and that the pain was no longer perceived by the subject almost immediately following application of the composition, respectively. For example, in these instances the improvement of pain was judged to be 4+ (90-100%) according to the above mentioned scale for clinical evaluation.

### Example 1: Aqueous Compositions

A typical aqueous solution comprising a tetrapeptide derivative according to the invention was formulated as follows:

An endomorphin-2 tetrapeptide derivative according to the invention (0.5 g) was dissolved in 99.5 ml of a solution prepared from 40 parts water, 40 parts ethanol and 20 parts propylene glycol by volume (hereinafter referred to as “WEP442”). The aqueous solution thus formulated contained 0.5% (w/v) tetrapeptide derivative in a solution composition. Under similar conditions aqueous solution compositions containing 0.1% to 2% (w/v) endomorphin-2 tetrapeptide derivative of the invention were readily formulated.

As an illustrative example, N-Ac-Tyr-Pro-Phe-Phe-NH₂ (0.5 g) (SEQ ID NO: 23), was dissolved in 99.5 ml of WEP442 solution. The solution thus formulated contained 0.5% (w/v) N-Ac-Tyr-Pro-Phe-Phe-NH₂ (SEQ ID NO: 23) in WEP442 aqueous solution composition. Under similar conditions, solution compositions containing N-Ac-Tyr-Pro-Phe-Phe-NH₂ (SEQ ID NO: 23) ranging from 0.1% to 2% (w/v) were readily formulated.

As a further illustration, and under the same conditions, solution compositions containing 0.1% to 2%, e.g., 0.5% (w/v) N-Pa-Tyr-Pro-Phe-Phe-NH₂ (SEQ ID NO: 64) in WEP442 were prepared.

For example, solution compositions containing 0.1%-2% (w/v) in WEP442 of the following tetrapeptide derivatives were readily formulated under the same conditions described above:
(a) Endomorphin-2 and tetrapeptide derivatives thereof: Tyr-Pro-Phe-Phe-NH₂ (SEQ ID NO: 1); Tyr-Pro-Phe-Phe-NHNH₂ (SEQ ID NO: 10); N-Ac-Tyr-Pro-Phe-Phe-NH₂ (SEQ ID NO: 23); N-Ac-Tyr-Pro-Phe-Phe-NHNH₂ (SEQ ID NO: 31); N-Pa-Tyr-Pro-Phe-Phe-NH₂ (SEQ ID NO: 64); N-Pa-Tyr-Pro-Phe-Phe-NHNH₂ (SEQ ID NO: 71); N-Pg-Tyr-Pro-Phe-Phe-NH₂ (SEQ ID NO: 44); N-Pg-Tyr-Pro-Phe-Phe-NHNH₂ (SEQ ID NO: 51); N-Bz-Tyr-Pro-Phe-Phe-NH₂ (SEQ ID NO: 84); N-Bz-Tyr-Pro-Phe-Phe-NHNH₂; N-Ac-Tyr-Pro-Phe-Phe-NHCH₃ (SEQ ID NO: 24); N-Ac-Tyr-Pro-Phe-Phe-NHNHAc (SEQ ID NO: 38); N-Pa-Tyr-Pro-Phe-Phe-NHCH₃ (SEQ ID NO: 65); N-Pa-Tyr-Pro-Phe-Phe-NHNHAc (SEQ ID NO: 78); N-Pg-Tyr-Pro-Phe-Phe-NHCH₃ (SEQ ID NO: 45); N-Pg-Tyr-Pro-Phe-Phe-NHNHAc (SEQ ID NO: 58); N-Bz-Tyr-Pro-Phe-Phe-NHCH₃ (SEQ ID NO: 85); N-Bz-Tyr-Pro-Phe-Phe-NHNHAc (SEQ ID NO: 98); N-Ac-Tyr-Pro-Phe-Phe-NHOH (SEQ ID NO: 43); N-Pa-Tyr-Pro-Phe-Phe-NHOH (SEQ ID NO: 83); N-Pg-Tyr-Pro-Phe-Phe-NHOH (SEQ ID NO: 63); AND N-Bz-Tyr-Pro-Phe-Phe-NHOH (SEQ ID NO: 103).
(b) Endomorphin-1 and tetrapeptide derivatives thereof: Tyr-Pro-Trp-Phe-NH₂; N-Ac-Tyr-Pro-Trp-Phe-NH₂; N-Pa-Tyr-Pro-Trp-Phe-NH₂; N-Pg-Tyr-Pro-Trp-Phe-NH₂ (SEQ ID NOs: 2, and 272-274, respectively).

### Example 2: Anhydrous Solution Compositions

A typical anhydrous solution composition comprising an endomorphin-2 tetrapeptide derivative according to the invention was formulated as follows.

An endomorphin-2 tetrapeptide derivative according to the invention (0.5 g) was dissolved in 99.5 ml of a solution prepared from 70 parts ethanol and 30 parts propylene glycol by volume (hereinafter referred to as “EP73”). The anhydrous solution thus formulated contained 0.5% (w/v) of an endomorphin-2 tetrapeptide derivative of the invention. Under similar conditions, anhydrous solution compositions containing 0.01% to 2% (w/v) of an endomorphin-2 tetrapeptide derivative of the invention were readily formulated.

As an illustration, N-Ac-Tyr-Pro-Phe-Phe-NH₂ (0.7 g) (SEQ ID NO: 23) was dissolved in 99.3 ml of EP73 solution. The anhydrous solution composition thus formulated contained 0.7% (w/v) N-Ac-Tyr-Pro-Phe-Phe-NH₂ (SEQ ID NO: 23) in EP73 solution. Under similar conditions, anhydrous solution compositions containing 0.1% to 2% (w/v) N-Ac-Tyr-Pro-Phe-Phe-NH₂ (SEQ ID NO: 23) were readily formulated.

For example, the anhydrous solution compositions containing 0.1-2% (w/v) in EP73 of the following tetrapeptide derivatives were readily formulated under the same conditions described above:
(a) Endomorphin-2 and tetrapeptide derivatives thereof: Tyr-Pro-Phe-Phe-NH₂ (SEQ ID NO: 1); N-Ac-Tyr-Pro-Phe-Phe-NH₂ (SEQ ID NO: 23); N-Pa-Tyr-Pro-Phe-Phe-NH₂ (SEQ ID NO: 64); N-Pg-Tyr-Pro-Phe-Phe-NH₂ (SEQ ID NO: 44); N-Ac-Tyr-Pro-Phe-Phe-NHNH₂ (SEQ ID NO: 31); N-Ac-Tyr-Pro-Phe-Phe-NHOH (SEQ ID NO: 43); N-Pa-Tyr-Pro-Phe-Phe-NHNH₂ (SEQ ID NO: 71); N-Pg-Tyr-Pro-Phe-Phe-NHNH₂ (SEQ ID NO: 51); N-Bz-Tyr-Pro-Phe-Phe-NHNH₂ (SEQ ID NO: 91); and N-Bz-Tyr-Pro-Phe-Phe-NHOH (SEQ ID NO: 103).
(b) Endomorphin-1 and tetrapeptide derivatives thereof: Tyr-Pro-Trp-Phe-NH₂; N-Ac-Tyr-Pro-Trp-Phe-NH₂; N-Pa-Tyr-Pro-Trp-Phe-NH₂; and N-Pg-Tyr-Pro-Trp-Phe-NH₂ (SEQ ID NOs: 2, and 272-274, respectively).

### Example 3: Anhydrous Cream Compositions

A typical anhydrous cream composition containing a tetrapeptide derivative of the present invention was formulated as follows.

An endomorphin-2 tetrapeptide derivative of the invention (0.5 g) was dissolved in warm propylene glycol (20 g) and oleyl lactate (30 g). The solution thus prepared was mixed with a melted mixture of beeswax (5 g), glyceryl monostearate ( ) PEG-40 stearate ( ) and shea butter (34.5 g) to obtain an anhydrous cream composition. The anhydrous cream composition thus formulated contained 0.5% (w/w) of the endomorphin-2 tetrapeptide derivative.

Under the same conditions, anhydrous cream compositions containing 0.1%-2% (w/w) of an endomorphin-2 tetrapeptide derivative of the invention were readily formulated.

For example, under the same conditions, anhydrous cream compositions containing 0.1%-2%, e.g., 0.5% (w/w) N-Ac-Tyr-Pro-Phe-Phe-NH₂(SEQ ID NO: 23) were readily formulated.

Alternative anhydrous cream compositions containing an endomorphin-2 tetrapeptide derivative of the invention were also formulated as follows.

An endomorphin-2 tetrapeptide derivative of the invention (0.5 g) was dissolved in warm propylene glycol (20 g) and oleyl lactate (15 g). The solution thus prepared was mixed with a melted mixture of sorbitan sesquioleate 0 and shea butter (49.5 g). The anhydrous cream composition thus formulated contained 0.5% (w/w) of the endomorphin-2 tetrapeptide derivative of the invention. Under the same conditions, anhydrous cream compositions containing 0.1%-2% (w/w) of an endomorphin-2 tetrapeptide derivative of the invention were readily formulated.

For example, under the same conditions, anhydrous cream compositions containing 0.1%-2%, e.g., 0.5% (w/w)N-Ac-Tyr-Pro-Phe-Phe-NH₂(SEQ ID NO: 23) were readily formulated.

In particular, anhydrous cream compositions containing 0.1%-2% (w/w) of the following tetrapeptide derivatives were formulated as described above:
(a) endomorphin-2 and tetrapeptide derivatives thereof: Tyr-Pro-Phe-Phe-NH₂; N-Ac-Tyr-Pro-Phe-Phe-NH₂; N-Pa-Tyr-Pro-Phe-Phe-NH₂; and N-Pg-Tyr-Pro-Phe-Phe-NH₂ (SEQ ID NOs: 1, 23, 64, and 44, respectively).
(b) endomorphin-1 and tetrapeptide derivatives thereof: Tyr-Pro-Trp-Phe-NH₂; N-Ac-Tyr-Pro-Trp-Phe-NH₂; N-Pa-Tyr-Pro-Trp-Phe-NH₂; and N-Pg-Tyr-Pro-Trp-Phe-NH₂ (SEQ ID NOs: 2, and 272-274, respectively).

### Example 4: Aqueous Cream or Emulsion Composition

A typical aqueous cream or oil-in-water emulsion composition comprising a tetrapeptide derivative according to the present invention was formulated as follows.

N-Ac-Tyr-Pro-Phe-Phe-NH₂ (0.7 g) (SEQ ID NO: 23) was dissolved in warm propylene glycol (20 ml) and oleyl lactate (20 ml). The solution thus obtained was mixed with an aqueous cream base or oil-in-water emulsion (59.3 g). The composition thus formulated contained 0.7% (w/w) N-Ac-Tyr-Pro-Phe-Phe-NH₂ (SEQ ID NO: 23). Under similar conditions, aqueous creams or oil-in-water emulsion scontaining 0.1-2% (w/w) N-Ac-Tyr-Pro-Phe-Phe-NH₂ (SEQ ID NO: 23) were readily formulated.

For example, aqueous creams or oil-in-water emulsions containing 0.1-2% (w/w) of the following tetrapeptide derivatives were readily formulated as described above:
(a) Endomorphin-2 and tetrapeptide derivatives thereof: Tyr-Pro-Phe-Phe-NH₂; N-Ac-Tyr-Pro-Phe-Phe-NH₂; N-Pa-Tyr-Pro-Phe-Phe-NH₂; and N-Pg-Tyr-Pro-Phe-Phe-NH₂ (SEQ ID NOs: 1, 23, 64, and 44, respectively).
(b) Endomorphin-1 and tetrapeptide derivatives thereof: Tyr-Pro-Trp-Phe-NH₂; N-Ac-Tyr-Pro-Trp-Phe-NH₂; N-Pa-Tyr-Pro-Trp-Phe-NH₂; and N-Pg-Tyr-Pro-Trp-Phe-NH₂ (SEQ ID NOs: 2, and 272-274, respectively).

### Example 5: Systemic Composition

For injection administration, an endomorphin-2 tetrapeptide derivative, such as N-Ac-Tyr-Pro-Phe-Phe-NH₂ (SEQ ID NO: 23) in fine powder form (one gram) was mixed with water (100 ml). The solution thus obtained was sterilized in injection vials at 100° C. for 30 minutes. The sterilized compositions thus obtained contained 1% (w/v) or 10 mg/ml of N-Ac-Tyr-Pro-Phe-Phe-NH₂ (SEQ ID NO: 23) suitable for intra-articular, intralesional, or subcutaneous injection, or other systemic administration.

For under the tongue administration (sublingual), a tetrapeptide derivative, such as N-Ac-Tyr-Pro-Phe-Phe-NH₂ (SEQ ID NO: 23) in fine powder form was used directly under tongue without mixing with any other ingredient(s).

For example, aqueous compositions formulated for systemic administration comprising 0.5%-1% (w/v) of following endomorphin-2 and tetrapeptide derivatives thereof of the invention were formulated as described above: Tyr-Pro-Phe-Phe-NH₂; N-Ac-Tyr-Pro-Phe-Phe-NH₂; N-Pa-Tyr-Pro-Phe-Phe-NH₂; N-Pg-Tyr-Pro-Phe-Phe-NH₂; N-Bz-Tyr-Pro-Phe-Phe-NH₂; and N-Bz-Tyr-Pro-Phe-Phe-NHNH₂ (SEQ ID NOs: 1, 23, 64, 44, 84, and 91, respectively).

### Example 6: Analgesic Effect on Viral Infection

A male subject, age 93, had herpes zoster on a temporal branch of the tri-geminal nerve with severe clinical pain involving the areas of left temporal, left forehead, left scalp, left neck, and left ear canal. Touching the left anterior scalp hair provoked severe pain.

The subject topically administered an anhydrous composition containing 0.5% (w/v) N-Ac-Tyr-Pro-Phe-Phe-NH₂ (SEQ ID NO: 23) in EP73, formulated as described in Example 2, by applying a few drops to wet the skin surface. Within about a minute of topical application, the pain disappeared completely. The pain relief lasted for up to 1 hour. Multiple repeated topical applications of the anhydrous composition containing 0.5% (w/v) N-Ac-Tyr-Pro-Phe-Phe-NH₂ (SEQ ID NO: 23) in EP73 gave the same analgesic result with 4+ relief of pain according to clinical evaluation, and also for longer periods of up to 6 hours.

The above results show that endomorphin-2 tetrapeptide derivatives of the invention can be used to treat pain caused by viral infections.

### Example 7: Analgesic Effect on Shoulder Pain

A female subject, age 52, had chronic right shoulder pain. The subject was unable to lift her arm above chest level. The subject topically applied an anhydrous solution composition containing 0.5% (w/v) N-Ac-Tyr-Pro-Phe-Phe-NH₂(SEQ ID NO: 23) in EP73, formulated as described in the Example 2, on her right shoulder at night. The next morning, 70% relief of pain was observed. The subject re-applied the composition at bedtime. The following morning, 85-90% relief was observed and she was able to lift her arm above her head with only minor discomfort.

The above result shows that endomorphin-2 tetrapeptide derivatives of the invention can be used to treat inflammation, arthritis, pain, and other nerve disorders.

### Example 8: Analgesic Effect on Knee Pain

A female subject, age 52, had chronic subpatellar and peripheral patellar pain in her knees.

The subject topically applied an anhydrous composition containing 0.7% (w/v) N-Ac-Tyr-Pro-Phe-Phe-NH₂ (SEQ ID NO: 23) in EP73, formulated as described in Example 2, at bedtime. The next morning, she observed about 50% relief of pain. The subject topically re-applied the same composition under a plastic film wrap for one hour, after which she observed about 95% relief of pain.

The above result shows that endomorphin-2 tetrapeptide derivatives of the invention can be used to treat inflammation, arthritis, pain and other nerve disorders.

### Example 9: Analgesic Effect on Ankle Pain

A female subject, age 52, had chronic arthritic pain of her left ankle. She was previously treated with prescription corticosteroid injections.

The subject topically applied an anhydrous composition containing 0.5% (w/v) N-Ac-Tyr-Pro-Phe-Phe-NH₂ (SEQ ID NO: 23) in EP73, formulated as described in Example 2, at bedtime. The next morning, she observed approximately 90% improvement of pain. The subject topically re-applied the same composition at bedtime. The following entire day, she had about 95% pain improvement.

The above result shows that endomorphin-2 tetrapeptide derivatives of the invention can be used to treat inflammation, arthritis, pain and other nerve disorders.

### Example 10: Analgesic Effect on Wrist Pain

A female subject, age 53, had sub-acute pain of her left wrist, which she twisted while lifting a child.

The subject topically applied an anhydrous composition containing 0.7% (w/v) N-Ac-Tyr-Pro-Phe-Phe-NH₂ (SEQ ID NO: 23) in EP73, formulated as described in Example 2, before bedtime. The next morning, about 70% relief of pain was observed. The subject topically re-applied the same composition under plastic film wrap for 2 hours, and the wrist pain was completely eradicated. The subject was free of any wrist pain and the improvement was 100% as judged by clinical evaluation.

The above result shows that endomorphin-2 tetrapeptide derivatives of the invention can be used to treat inflammation, arthritis, pain and other nerve disorders.

### Example 11: Analgesic Effect on Headache

A female subject, age 55, developed an ordinary type of headache in the early afternoon. Such headaches usually would persist for several hours, being relieved incompletely by treatment with oral drugs, such as ibuprofen and/or acetaminophen. In this instance, she topically applied an anhydrous composition containing 1% (w/v) N-Ac-Tyr-Pro-Phe-Phe-NH₂(SEQ ID NO: 23) in EP73 to the frontal and temporal areas. Within minutes, the headache pain disappeared completely and the headache was alleviated over the next 18 hours.

The above result shows that endomorphin-2 tetrapeptide derivatives of the invention can be used to treat headache pain.

### Example 12: Analgesic Effect on Severe Migraine Headache

A female subject, age 46, had a 10-15 year history of severe migraine attacks, occurring every 1-3 weeks. One day, at approximately 8 AM, visual aura and pain in the frontal scalp, forehead and frontal sinuses began and became very severe within 5-10 minutes. Thereafter, the subject topically applied a composition containing 1% (w/v) N-Ac-Tyr-Pro-Phe-Phe-NH₂(SEQ ID NO: 23) in WEP442 to the involved area. About 6-7 minutes later, when she was prepared to make another topical application, the migraine pain and all other symptoms suddenly disappeared completely and did not return. Three (3) days later she remained free of any migraine headache and symptoms.

The above result shows that endomorphin-2 tetrapeptide derivatives of the invention has can be used to treat migraine headache pain.

### Example 13: Analgesic Effect on Chronic Migraine Headache

A female subject, age 51, had a 10-11 year history of severe migraine attacks with pain affecting the post-auricular and occipital scalp areas. One late afternoon, the subject developed severe pain in those areas, accompanied by feelings of vertigo and nausea. The subject topically applied a composition containing 1% (w/v) N-Ac-Tyr-Pro-Phe-Phe-NH₂, (SEQ ID NO: 23) in WEP442 to the entire occipital scalp, including the peri-auricular areas. Within about 5 minutes, all pain and other symptoms disappeared. There was no recurrence of any migraine headache during the next week.

The above result shows that endomorphin-2 tetrapeptide derivative of the invention can be used to treat migraine headache pain.

### Example 14: Analgesic Effect on Osteoarthritic Knees and Lower Back

A male subject, age 94, had severe knee pain for many years and lower back pain for approximately one year from osteoarthritis, and in both cases, the pain was partially relieved by oral opiates. One day before bedtime, the subject took a low dose of an opiate, but the next morning, he still developed substantial pain in the knees and moderate pain in the lower back.

The subject topically applied approximately 60 ml of a solution composition containing 1% (w/v) N-Ac-Tyr-Pro-Phe-Phe-NH₂ (SEQ ID NO: 23) in WEP442 to the whole body, excluding only the scalp, face and soles. Over the next 2 hours, the pain in both knees and the lower back diminished substantially, and after 4 hours the pain in both knees and the lower back diminished to very low level. This lowered degree of pain was maintained for about 7 hours.

The above results show that endomorphin-2 tetrapeptide derivatives of the invention can be used to treat arthritis pain.

### Example 15: Analgesic Effect on Injured Fingers

A male subject, age 17, jammed his middle finger causing severe pain and swelling (inflammation). The subject topically applied a composition containing 1% (w/v) N-Ac-Tyr-Pro-Phe-Phe-NH₂ (SEQ ID NO: 1) in WEP442 to the affected finger. The pain and much of the swelling disappeared almost immediately after topical application.

The above result shows that endomorphin-2 tetrapeptide derivatives of the invention can be used to treat pain and inflammation.

### Example 16: Analgesic Effect on Neck and Shoulder Pain

A male subject, age 47, had a history of neck and shoulder pain for the past 4 years. The subject could not move his neck without having a high level of pain and discomfort. The subject topically applied a composition containing 0.5% (w/v) N-Ac-Tyr-Pro-Phe-Phe-NH₂(SEQ ID NO: 23) in WEP442 to the affected area of the skin. Within 2 minutes after topical application, the subject had approximately 90% relief of pain in his shoulder and neck, and had a full range of neck motion with only minimal discomfort. The pain relief lasted for more than 2 hours.

The above result shows that endomorphin-2 tetrapeptide derivatives of the invention can be used to treat pain, arthritis and inflammation.

### Example 17: Analgesic Effect on Osteoarthritic Knees

A male subject, age 92, had severe osteoarthritis of both knees with pain, inflammation and edema for four years. The subject underwent prior therapy, including intra-articular injections of corticosteroids and hyaluronic acid as well as celecoxib (Celebrex), administered orally (200 mg) twice daily. Such therapy provided only mild transitory relief of knee pain and edema, and edema of the lower legs.

The subject administered N-Pg-Tyr-Pro-Phe-Phe-NH₂ (SEQ ID NO: 44) powder (50 mg) under the tongue (sublingually), which dissolved in saliva and absorbed slowly for about 30 minutes. Within 20 minutes, the pain disappeared. The analgesic effect lasted for about 2 hours. The analgesic effect was judged to be about 4+ by clinical evaluation.

Under the same conditions, N-Pa-Tyr-Pro-Phe-Phe-NH₂ (SEQ ID NO: 64) powder provided an analgesic effect of about 3+ as judged by clinical evaluation. Also under the same conditions, N-Ac-Tyr-Pro-Phe-Phe-NH₂ (SEQ ID NO: 23) powder provided an analgesic effect of about 3+ as judged by clinical evaluation. Repeated application of the tetrapeptide derivatives provided 4+ efficacy as judged by clinical evaluation.

The above results show that endomorphin-2 tetrapeptide derivatives of the invention can be used to treat inflammation, arthritis, pain and other immune and nerve disorders.

### Example 18: Analgesic Effect on Arthritic Hip

A male subject, age 83, had severe arthritis of the left hip with pain and inflammation for 6 months.

The subject topically applied a composition containing 0.7% (w/v) N-Ac-Tyr-Pro-Phe-Phe-NH₂ (SEQ ID NO: 23) in WEP442 to the left hip and covered the hip with plastic wrap overnight. The pain reduced about 50% in 8 hours. The samen procedure was repeated the following day. The hip pain disappeared completely after another 8 hours of topical application. The analgesic effect observed was 100% improvement as judged by clinical evaluation.

The above results show that endomorphin-2 tetrapeptide derivatives of the invention can be used to treat inflammation, arthritis, pain and other immune and nerve disorders.

### Example 19: Analgesic Effect on Psoriatic Arthritis of Fingers

A female subject, age 57, had extensive plaque psoriasis and painful arthritis of the interphalangeal joints of both index fingers, left thumb and left 5^{th} finger. The pain was compromising her work performance as an esthetician. The subject topically applied a composition containing 1% (w/v) N-Ac-Tyr-Pro-Phe-Phe-NH₂ (SEQ ID NO: 23) in WEP442 each day for an interval of one month, and all arthritic pain of the finger was gone within about 30 seconds following each application. Pain relief lasted for about 6 hours.

The above result shows that endomorphin-2 tetrapeptide derivatives of the invention can be used to treat psoriatic arthritis.

### Example 20: Analgesic Effect on Arthritic Pain of Upper Back

A female subject, age 56, had arthritic pain in her upper back and associated paresthesia of her fingers, described as feelings of tingling and “pins and needles”. These symptoms had been present for about 1 year. Daily topical applications of a composition containing 1% (w/v) N-Ac-Tyr-Pro-Phe-Phe-NH₂ (SEQ ID NO: 23) in WEP442 to the upper back relieved the upper back pain completely within 3-4 minutes. Pain relief lasted for 10-12 hours. In contrast, the paresthesia of the fingers persisted without any discernible change. Repeated applications for 10-12 days gave the same results.

The above result shows that endomorphin-2 tetrapeptide derivatives of the present can be used to treat arthritis pain.

### Example 21: Analgesic Effect on Ankle Sprain

A female subject, age 47, suffered a sprain of the left ankle after stepping on a stone while hiking, twisting the ankle inwardly. Ten days later, after wearing an elastic ankle support daily, pain from the injury persisted. The subject topically applied an anhydrous composition containing 0.7% (w/v) N-Ac-Tyr-Pro-Phe-Phe-NH₂ (SEQ ID NO: 23) in EP73, formulated as described in Example 2. Within 3-4 minutes, the pain was relieved almost completely, at which time the composition was again applied. Within about 5 minutes, there was complete relief of all discomfort. Upon arising the next morning, the subject experienced mild ankle pain, whereupon the composition was again applied. Within about 5 minutes, no discomfort was discernible. The subject was discomfort free thereafter.

This result indicates that endomorphin-2 tetrapeptide derivatives of the invention can be used to treat pain from joint sprains.

### Example 22: Analgesic Effect on Medial Tibial Stress Syndrome (MTSS), Also Known as Tibial Periostitis

A small statured female, age 62, caretaker of an over-weight disabled elderly woman, every evening suffers pain from MTSS of the left lower leg that is due to repeated use of the left leg to lift her patient throughout the day. Her sleeping has been repeatedly disturbed by the pain. A single topical application to the anterior lower leg of a composition comprising 1% (w/v) N-Ac-Tyr-Pro-Phe-NH₂ (SEQ ID NO: 23) in WEP442 in the early evening completely relieved the pain within 1 minute. No pain occurred until the next evening after working with her patient, at which time she again applied the 1% solution composition. She followed this sequence of procedures daily for one week work shift with the same result of rapid absolute relief of pain. During the week of being off from work, the MTSS is absent.

This therapeutic result indicates that endomorphin-2 tetrapeptide derivatives of the invention can be used to treat painful periostitis and the like.

### Example 23: Analgesic Effect on Lower Back Pain

A female subject, age 59, working as a house cleaner, complained of moderate lower back pain at the end of a work day. Topical application to the lower back of a composition containing 1% (w/v) of N-Ac-Pro-Phe-Phe-NH₂ (SEQ ID NO: 23) in WEP442 was found to provide almost complete relief of pain within 1-2 minutes. She found that such applications provided pain relief on 7-8 occasions over a three week period.

This result indicates that endomorphin-2 tetrapeptide derivatives of the invention can be used to relieve lower back pain caused by physical labor.

### Example 24: Analgesic Effect on Severe Headaches

A female subject, age 33, had very severe headaches and associated nausea and vomiting, which occurred 1-2 times weekly. Topical applications at the onset of symptoms of a composition containing 1% (w/v) of N-Ac-Tyr-Phe-Phe-NH₂ (SEQ ID NO: 23) in WEP442 were found to completely relieve pain and any associated symptoms within 3-4 minutes. Repeated uses over a 3 month period continually provided such complete relief. Over this interval, the frequency of headaches decreased to every 7-10 days, and the intensity of pain at the onset of each episode was diminished.

The above result shows that endomorphin-2 tetrapeptide derivatives of the invention can be used to treat, reduce, and prevent the occurrence of severe headaches.

### Example 25: Analgesic Effect on Severe Muscle Soreness

A male subject, age 40, had disabling pain in his hands and fingers the next morning after a day of intense exertion of the hands and fingers from using hand screw drivers over a 12-hour period during a building construction job. The intensity of pain was so great that he was unable to lace up his work boots. Topical application in a hand-wash manner of a composition containing 1% (w/v) of N-Ac-Tyr-Pro-Phe-Phe-NH₂ (SEQ ID NO: 23) in WEP442 provided instantaneous, complete relief of pain. The pain did not recur at all.

The above result shows that endomorphin-2 tetrapeptide derivatives of the present invention can be used to treat tissue soreness following physical stress.

### Example 26: Analgesic Effect on Painful Inflammation of Hallux Valgus (Bunion)

A female subject, age 45, had hallux valgus of the right 1^{st} metatarsal-proximal phalangeal joint for a duration of 5-6 years. Pain in the joint was episodic and associated with inflammation. On a day when the joint was very painful, showed erythematous and was swollen, she topically applied composition containing 1% (w/v) of N-Ac-Tyr-Pro-Phe-Phe-NH₂ (SEQ ID NO: 23) in WEP442 to the joint. Within about 15 minutes, all pain was gone. A second application of the solution was made that evening, even though there was no pain. The next morning, the erythema was gone, the swelling had subsided, and there was no pain. Such improvement lasted over the next 2 weeks.

The above result shows that endomorphin-2 tetrapeptide derivatives of the invention can be used to treat signs and the symptom of pain from hallux valgus.

### Example 27: Analgesic Effect on Dental Pain

A female subject, age 38, experienced substantial pain in an upper left retro-molar that started 3 hours after having a dental filling procedure done on the tooth. The pain became more intense over the ensuing hours, at which time she applied to the tooth and gum using a cotton tipped applicator a composition containing 1% (w/v) of N-Ac-Tyr-Pro-Phe-Phe-NH₂ (SEQ ID NO: 23) in WEP442. The pain ceased completely within 1 minute, and lasted for about 3 hours. Re-application of the composition provided the same relief for the same duration.

The above result shows that endomorphin-2 tetrapeptide derivatives of the invention can be used to treat tooth pain.

### Example 28: Analgesic Effect on Dental Extraction Pain

A female subject, age 57, had substantial pain in the left mandibular jaw that started about 3 hours after extraction of a left mandibular molar tooth. Topical application to the left facial-lower jaw skin areas of a composition containing 1% (w/v) of N-Ac-Tyr-Pro-Phe-Phe-NH₂ (SEQ ID NO: 23) in WEP442 provided complete relief of pain in less than 1 minute. Only negligible discomfort was perceptible thereafter.

The above result indicates that endomorphin-2 tetrapeptide derivative of the invention can be used to treat jaw pain following tooth extraction.

Example 29: Analgesic Effect on Dental Extraction Pain

A male subject, age 88, had substantial pain in the right maxillary jaw that started about 2 hours after extraction of a right maxillary tooth. Topical application to the right facial maxillary area of a composition containing 1% (w/v) of N-Ac-Tyr-Pro-Phe-Phe-NH₂ (SEQ ID NO: 23) in WEP442 provided complete relief of pain within 1 minute. Only negligible discomfort, perceived by touching the extraction site with the tongue, was detectable thereafter.

The above result shows that endomorphin-2 tetrapeptide derivatives of the invention can be used to treat jaw pain following tooth extraction.

Example 30: Analgesic Effect on Sunburn Pain

A female subject, age 54, with severe sunburn of her anterior legs the day after exposure to sunlight without sunscreen protection, topically applied a composition containing 1% (w/v) of N-Ac-Tyr-Pro-Phe-Phe-NH₂ (SEQ ID NO: 23) in WEP442 to both legs. The sunburn pain of the thighs was completely relieved almost instantly, and lasted for about 3 hours.

The above result shows that endomorphin-2 tetrapeptide derivatives of the invention can be used to relieve sunburn pain.

### Example 31: Analgesic Effect on Painful Inflammation Following Trauma

A female subject, age 54, experienced sharp pain in her right index finger while trying to loosen a screw-on lid of a fruit jar with her right hand. After about 10 minutes, the pain became throbbing in nature and very intense. The proximal interphalangeal became swollen and discolored (black and blue) from subcutaneous hemorrhage. Topical application at that time of a composition containing 1% (w/v) of N-Ac-Tyr-Pro-Phe-Phe-NH₂ (SEQ ID NO: 23) in WEP442 to the finger gave instantaneous total relief of pain. No pain returned. Discoloration of the joint faded over the next several days.

The above result shows that endomorphin-2 tetrapeptide derivatives of the invention can be used to relieve joint pain following physical injury.

### Example 32: Analgesic Effect on Knee Pain After Housework

A female subject, age 52, with discomforting knee pain in the evening after cleaning her own house for several hours topically applied a composition containing 1% (w/v) of N-Ac-Tyr-Pro-Phe-Phe-NH₂ (SEQ ID NO: 23) in WEP442. Total relief of pain occurred within 1 minute and lasted until bedtime 5-7 hours later after which a second application provided a pain-free night of sleep.

The above result shows that endomorphin-2 tetrapeptide derivatives of the invention can be used to relieve knee pain.

### Example 33: Analgesic Effect on Recurring Headaches

A female subject, age 35, with intense headaches after several hours of using a computer in her work as a real estate agent, topically applied to the forehead and temporal areas a composition containing 0.8% (w/v) of N-Ac-Tyr-Pro-Phe-Phe-NH₂ (SEQ ID NO: 23)0 in EP73, formulated as described in Example 2. Complete headache relief occurred within 1 minute. Such headaches, occurring 1-2 times weekly, were treated in the same manner over a 2 month interval, with headache pain being relieved each time.

The above result shows that endomorphin-2 tetrapeptide derivatives of the invention can be used to relieve headaches repeatedly over an extended period of time.

### Example 34: Analgesic Effect on Pain in Big Toe Following Trauma

A female subject, age 50, traumatized her right big toe while repositioning heavy furniture, and experienced disabling persistent pain of that toe. About 10 minutes after the trauma occurred, she topically applied a composition containing 1% (w/v) of N-Ac-Tyr-Pro-Phe-Phe-NH₂ (SEQ ID NO: 23) in WEP442 to the toe. Complete relief of pain occurred within 15-30 seconds. The pain did not return.

This result shows that endomorphin-2 tetrapeptide derivatives of the invention can be used to relieve pain of mechanically traumatized toes.

### Example 35: Analgesic Effect on Soreness of Subcutaneous Lipomas

A female subject, age 54, with many subcutaneous lipomas on her arms, torso and thighs that often become painfully sore after being bumped against objects during the course of daily activities, topically applied to tenderly sore lipomas a composition containing 1% (w/v) N-Ac-Tyr-Pro-Phe-Phe-NH₂ (SEQ ID NO: 23) in WEP442. Such applications were repeated multiple times over a period of two months. Each and every application promptly relieved all feelings of soreness within 1 minute.

These results show that endomorphin-2 tetrapeptide derivatives of the invention can be used to relieve soreness of subcutaneous lipomas.

### Example 36: Analgesic Effect on Pain of Elbow Bursitis

A male subject, age 67, had painful bursitis of the right elbow that impaired his performance as a golfer. The subject topically applied a composition containing 0.8% (w/v) of N-Ac-Tyr-Pro-Phe-Phe-NH₂ (SEQ ID NO: 23) in WEP442 to the right elbow on an afternoon and experienced complete loss of pain within about 2 minutes. Pain did not recur until the next morning when he again applied the solution to the elbow. Again, he experienced complete loss of pain promptly within about 2 minutes. He played golf later the same day and experienced no bursitis pain during the game.

The above result indicates that endomorphin-2 tetrapeptide derivatives of the invention can be used to relieve pain and discomfort of bursitis.

### Example 37: Analgesic Effect on Bruised Finger

A female subject, age 35, severely bruised her left 5^{th} finger while trying to close a basement window in her house. The pain was intense, and associated with subcutaneous hemorrhage. Topical application of a composition containing 1% (w/v) of N-Ac-Tyr-Pro-Phe-Phe-NH₂ (SEQ ID NO: 23) in WEP442 to the entire finger provided complete relief of pain almost instantaneously, i.e. within about 15 seconds.

The above result shows that endomorphin-2 tetrapeptide derivatives of the invention can be used to relieve severe pain of traumatized fingers.

### Example 38: Analgesic Effect on Gum Pain Due to Pressure From Denture

A male subject, age 94, with a severely sore site on the left lower gum due to localized pressure from ill-fitting dentures, applied using a fingertip a composition containing 1% (w/v) of H-Tyr-Pro-Phe-Phe-NH₂ (SEQ ID NO: 1) in WEP442 directly to the sore area when the dentures removed. The dentures were promptly re-inserted and within 5-10 seconds, all soreness was gone, and the relief lasted for about 7 hours. The dentures were removed and re-inserted 15 hours later. Within about 1 hour, soreness of the same area began to return and application of the above solution composition was repeated in the same way as the day before. All soreness again was alleviated within 5-10 seconds. The relief lasted again for about 7 hours.

The above result shows that endomorphin-2 tetrapeptide derivatives of the invention can be used to relieve pain and soreness of gums.

### Example 39: Analgesic Effect on Pain at Site of Subcutaneous Injection of Hyaluronic Acid

A female subject, age 57, experienced substantial pain in the zygomatic-facial areas, sites injected subcutaneously with a hyaluronic acid product (Restylane) six hours earlier for the cosmetic purpose of plumping up the skin. Topical application to the site of a composition containing 1% (w/v) of N-Ac-Tyr-Pro-Phe-Phe-NH₂ (SEQ ID NO: 23) in WEP442 completely relieved the pain within about 1 minute. The pain did not return.

The above result shows that endomorphin-2 tetrapeptide derivatives of the invention can be used to relieve pain following subcutaneous injections of cosmetic products, e.g., hyaluronic acid.

### Example 40: Treatment of Painful Extruded Intervertebral Cervical Disc 6-7 by Topical or Systemic Administration of Endomorphin-2

A male subject, age 93, had an extruded intervertebral cervical disc at disc 6-7, radiologically diagnosed several years earlier. Previous therapy undergone by the subject included epidural injections of corticosteroids. Within the past several months, the pain had intensified, along with a substantial degree of paresthesia and numbness of the fingers of both hands. A 5 cm×5 cm piece of gauze, saturated with a solution containing 0.5% (w/v) Tyr-Pro-Phe-Phe-NH₂ (SEQ ID NO: 1) in WEP442, was positioned over cervical vertebra 7 and covered with a plastic film, which was then taped in place. There was complete relief of symptoms after 8 hours, at which time the gauze was removed. Relief of pain and paresthesia lasted for 1-2 days. Thereafter, the pain and paresthesia returned.

Several days later, a sterile aqueous solution containing Tyr-Pro-Phe-Phe-NH₂ (SEQ ID NO: 1) at a concentration of 3 mg/ml was injected subcutaneously at the site over cervical vertebra 7. The next day, the pain and paresthesia diminished substantially, and the injection was repeated again 24 hours later. About 1 week later, pain and paresthesia were negligible, but returned within 2-3 weeks. Such injections were repeated 3 times at approximately monthly intervals with the same beneficial results. However, pain and paresthesia were not cured, and recurring within 2-3 weeks.

The above results show that both topical and systemic administration of endomorphin-2 according to the invention are effective in relieving severe pain and paresthesia from extrusion of cervical vertebrate discs.

### Example 41: Analgesic Effect on Migraine Headaches and TMJ (Temporomandibular Joint) Pain

A female subject, age 48, with severe migraine headaches associated with nausea and vomiting that had been occurring 3-4 times per month over the past 9-10 years, topically applied a composition containing 1% (w/v) of N-Ac-Tyr-Pro-Phe-Phe-NH₂ (SEQ ID NO: 23) in WEP442 to the forehead, brow, eyelids and temporal areas at the onset of headaches. Complete relief of pain and other symptoms occurred within 2-3 minutes on each occasion. The frequency of episodes diminished over the next two months, and the subject reported that she experienced no headaches for at least a 5 week period.

The same female subject, age 48, also suffered pain from TMJ syndrome involving the left side joint for about 6 years. Attempts to alleviate the pain, e.g. cushioning mouth pieces worn during the night, failed. Topical applications to the left side of the face of a composition containing 0.5% (w/v) of N-Ac-Tyr-Pro-Phe-Phe-NH₂ (SEQ ID NO: 23) in WEP442 rapidly and completely alleviated pain for more than 8 hours. Increasing longer periods of relief have occurred over 5-6 weeks. She has reported that there was no more pain in the affected joint over a period of about one month.

The above result shows that topical administration of endomorphin-2 tetrapeptide derivatives of the invention can be used to relieve pain from temporomandibular joint disorder (TMJ) and migraine headaches after topical administration.

### Example 42: Analgesic Effect on Knee Pain Following Twisting of Leg

A male subject, age 40, twisted his left leg during work on building construction, causing substantial pain in the left knee. The pain persisted into the late evening, preventing him from falling asleep. After about 20 minutes in bed, he topically applied to the left knee a composition containing 1% (w/v) of N-Ac-Tyr-Pro-Phe-Phe-NH₂ (SEQ ID NO: 23) in WEP442, which provided complete relief of pain in less than 1 minute thus allowing him to fall asleep pain free. He was pain free the next morning and thereafter.

The above result shows that topical administration of endomorphin-2 tetrapeptide derivatives of the invention can be used to relieve knee pain.

Example 43: Comparative Analgesic Efficacy of a Tetrapeptide Derivative in Different Compositions

The representative tetrapeptide derivative N-Ac-Tyr-Pro-Phe-Phe-NH₂ (SEQ ID NO: 23) was formulated as a 0.5% (w/v) aqueous composition in WEP442; 0.5% (w/w) anhydrous cream composition; and 0.5% (w/w) aqueous cream composition as described in Examples 1, 3, and 4. A female subject, age 48, with TMJ syndrome and a female subject, age 57, with psoriatic arthritis of the fingers compared the analgesic efficacy of the three compositions on their painful conditions. Each concluded that (i) the aqueous solution composition provided almost instantaneous and complete relief of pain lasting for 7-8 hours; (ii) the aqueous cream composition provided substantial, but not complete, relief of pain that occurred only after 5-6 minutes and lasted for 3-4 hours; and (iii) the anhydrous cream composition provided slow, mild relief of pain, lasting for 2-3 hours.

The above results show that efficacy of an endomorphin-2 tetrapeptide derivative of the invention can vary depending on the type of composition, formulation, etc.

### Example 44: Analgesic Effect on Headache

A female subject, age 55, experienced a headache that began on the right temporofrontal area. Within about a half hour the headache pain area expanded to include the left temporofrontal area and both orbital areas. The intensity of pain increased simultaneously to a degree that caused her to remove herself from her sedentary occupation. Available to her was a composition containing 1% (w/v) of N-Pg-Tyr-Pro-Phe-Phe-NH₂ (SEQ ID NO: 44) in WEP442, which she topically applied to all the above areas, keeping her eyes closed until the lids dried. Pain relief was detected after about 5 minutes, at which time a second application of the solution was made. Within another 5 minutes, pain relief was substantial, allowing her to return to her occupation. Total relief of pain occurred within about another 5 minutes. There was no recurrence of pain.

The above result shows that topical administration of endomorphin-2 tetrapeptide derivatives of the invention can be used to relieve headache pain.

### Example 45: Analgesic Effect on Osteoarthritic Knees

One gram of the tetrapeptide derivative N-Pg-Tyr-Pro-Phe-Phe-NH₂(SEQ ID NO: 44) was dissolved in propylene glycol (33 ml) and diisopropyl adipate (33 ml), and the resultant solution was mixed with an oil-in-water emulsion (33 g) to yield a light lotion containing 1% (w/w) of N-Pa-Tyr-Pro-Phe-Phe-NH₂ (SEQ ID NO: 44). This lotion was generously applied topically to the knees and lower legs of a male subject, age 94, having very severe osteoarthritis of the knees for many years. After topical application of the lotion to the lower legs and knees, plastic film was applied to cover the treated areas. The plastic film was removed 8 hours later. The foregoing procedure was repeated daily for 3 days at which time pain had subsided almost completely. Pain began to return after about 4 days.

The above result shows that endomorphin-2 tetrapeptide derivatives of the invention can be used to relieve arthritic pain following topical administration.

### Example 46: Analgesic Effect on Chronic Knee Pain From Post-Trauma

A female subject, age 56, had a history of physical trauma to the right knee in childhood. Onset of pain occurred in adulthood at approximately age 40, worsening to a level that compromised her performance as a hospital nurse. Topical application to the affected knee of a composition containing 1% (w/v) of N-Ac-Tyr-Pro-Phe-Phe-NH₂ (SEQ ID NO: 23) in WEP442 provided complete absence of pain during an 8 hours shift of work.

The above result shows that endomorphin-2 tetrapeptide derivatives of the invention can be used to relieve chronic knee pain following topical administration.

### Example 47: Analgesic Effect on Severe Headaches

A female subject, age 30, had severe frontal headaches occurring every 5-10 days. Topical application at the onset to frontal areas of a composition containing 1% (w/v) of N-Ac-Tyr-Pro-Phe-Phe-NH₂ (SEQ ID NO: 23) in WEP442 completely interrupted all pain within 3-4 minutes, and the headache was completely gone.

The above result shows that endomorphin-2 tetrapeptide derivatives of the invention can be used to relieve severe headache pain following topical administration.

### Example 48: Comparative Analgesic Efficacy of Psoriatic Arthritis Pain Involving Fingers and Wrists

A female subject, age 54, had severe generalized psoriasis, which had its onset at about the age of 16. Treatment of the subject over the years included methotrexate and other antimetabolites, and in recent years drugs known as biologic medications. While these agents have provided intervals of relief, none has provided a lasting benefit. Over the past decade, painful arthritis developed in both wrists and in numerous joints of the fingers. Four related tetrapeptide derivatives, 1% (w/v) solution compositions in vehicle WEP442, one at a time, were topically applied to the fingers and wrists of both hands. Analgesic efficacy of each was judged to be as follows.
(A) N-Ac-Tyr-Pro-Phe-Phe-NH₂ (SEQ ID NO: 23) provided rapid complete relief of pain, i.e. within 2-4 minutes, lasting for 6-7 hours. Efficacy was rated by the subject as 4+.
(B) N-Ac-Tyr-Pro-Phe-Phe-NHNH₂ (SEQ ID NO: 31) provided slow relief of pain, i.e. within about 10 minutes. Almost complete relief of pain occurred and lasted for 6-7 hours. Efficacy was rated by the subject as 3+ to 4+.
(C) N-Pg-Tyr-Pro-Phe-Phe-NH₂ (SEQ ID NO: 44) provided rapid relief of pain, i.e. within 2-4 minutes, and lasted for 6-7 hours. The subject rated analgesic efficacy as 4+.
(D) N-Pg-Tyr-Pro-Trp-Phe-NH₂ (SEQ ID NO: 274) an endomorphin-1 derivative provided slow, minimal relief of pain. Analgesic efficacy was judged by the subject to be 0 to 1+.

The above results show that endomorphin-2 tetrapeptide derivatives of the invention can be used to relieve psoriatic arthritis pain following topical administration. However, an endomorphin-1 tetrapeptide derivative did not provide any substantial relief of psoriatic arthritic pain following topical administration.

### Example 49: Topical Treatment of Severe Right Temporal Headache

A female subject, age 39, had an acute right temporal nauseating headache, which had a rapid onset over a 5-10 minute interval while attending a religious ceremony. She was given, by a nearby companion, a solution composition containing 1% (w/v) of N-Pg-Tyr-Pro-Phe-Phe-NH₂ (SEQ ID NO: 44) in WEP442, which she topically applied to the right temporal area, right closed eyelids, entire forehead and right eyebrow. Within 3-5 minutes, the headache subsided completely and did not return.

The above result shows that endomorphin-2 tetrapeptide derivatives of the invention can be used to relieve headache pain following topical administration.

### Example 50: Topical Treatment of Painful Tendonitis of Right Wrist and Right Thumb

A female subject, age 46, with painful tendonitis after playing tennis topically applied a composition containing 1% (w/v) of N-Pg-Tyr-Pro-Phe-Phe-NH₂ (SEQ ID NO: 44) in WEP442 to the right thumb and to the entire wrist. Within 5-10 minutes, all pain and discomfort from any hand movements were gone and did not return within a 24 hour follow up period. The above result shows that endomorphin-2 tetrapeptide derivatives of the invention can be used to relieve tendonitis pain following topical administration.

### Example 51: Analgesic Effect on Stress Induced Headache

A female subject, age 38, had a late morning developing headache involving the frontal, temporal and periocular areas. The onset of the headache was associated with emotional stress. Within two hours, the intensity of the headache became almost disabling, whereupon she applied a solution composition containing 1% (w/v) N-Pg-Ty-Pro-Phe-Phe-NH₂ (SEQ ID NO: 44) in WEP442 to the entire frontal and upper facial areas. Within about 5 minutes, the headache diminished in intensity. At that time, the solution was generously applied a second time. Within another 5 minute,s she was free of all headache pain, and the pain did not return after 24 hours. The analgesic effect was judged to be 100%.

### Example 52: Analgesic Effect on Headache

A female subject, age 34, had a headache that began at 2 p.m. in the afternoon after driving a car for about 3 hours. At the end of the drive, at about 3:30 p.m., she took two 500 mg acetaminophen tablets, which provided slight relief of pain. At 5:30 p.m., the headache pain was becoming more intense, at which time she topically applied a composition containing 1% (w/v) of N-Pg-Tyr-Pro-Phe-Phe-NH₂ (SEQ ID NO: 44) in WEP442 to the entire forehead and temporal areas. Within 30 seconds, all pain, and heavy feelings were completely gone. There was no recurrence of symptoms over the next 3 days of follow-up time.

The above result shows that topical application of endomorphin-2 tetrapeptide derivatives of the invention can provide rapid, complete relief of headache, and with greater efficacy than that of orally administered acetaminophen.

### Example 53: Evaluation of six (6) Tetrapeptide Derivatives for Comparative Analgesic Efficacy in Topical Treatment of Knee Osteoarthritis

A male subject, age 94, with chronic osteoarthritis of the knees participated in this study. Each tetrapeptide derivative was dissolved in WEP442 to make a 1% (w/v) solution. Early in the morning, the solution was applied to a knee, which was immediately wrapped in plastic film from a roll 5 inches wide. The film wrap was left in place for about 7-8 hours, and then it was removed. Such application was repeated at bedtime and removed several hours later during the night. Each 1% (w/v) solution of tetrapeptide derivative was so applied for 2 days and efficacy was evaluated by the subject. No topical treatment was made during an ensuing interval of 2-3 days, at which time treatment with a 1% (w/v) solution of another derivative was initiated and carried out as described above. In total, six tetrapeptide derivatives were evaluated for analgesic efficacy.

The following tetrapeptide derivatives were judged to provide 3+ to 4+ pain relief by clinical evaluation: Tyr-Pro-Phe-Phe-NH₂ (SEQ ID NO: 1), N-Pg-Tyr-Pro-Phe-Phe-NH₂ (SEQ ID NO: 44), N-Ac-Tyr-Pro-Phe-Phe-NH₂ (SEQ ID NO: 23), and N-Ac-Tyr-Pro-Phe-Phe-NHNH₂ (SEQ ID NO: 31).

The following tetrapeptide derivative was judged to provide 2+ to 3+ pain relief by clinical evaluation: N-Pa-Tyr-Pro-Phe-Phe-NH₂(SEQ ID NO: 64).

After the above evaluation period, maintenance treatment was done with a composition containing 1% (w/v) of N-Pg-Tyr-Pro-Phe-Phe-NH₂ (SEQ ID NO: 44) in WEP442 applied twice daily. The judgement of the subject had been that the knee pain was substantially less compared to what it was several months earlier.

The above results show that endomorphin-2 tetrapeptide derivatives of the invention can be used to relieve chronic arthritis pain following topical administration.

### Example 54: Analgesic Effect on “Hangover” Headache the Morning After Excessive Drinking

A male subject, age 26, with a “splitting” headache the morning after heavy drinking at a party the night before, topically applied to the entire head and neck a composition containing 1% (w/v) of N-Ac-Tyr-Pro-Phe-Phe-NHNH₂ (SEQ ID NO: 31) in WEP442. Within 1-2 minutes, the headache ceased, and the subject said he felt very good otherwise. He then drove his car approximately 90 miles to his residence.

The above result shows that endomorphin-2 tetrapeptide derivatives of the invention can be used to relieve hangover headache following topical administration.

### Example 55: Analgesic Effect on Headache Due to Sinusitis

A female subject, age 59, suffered from chronic recurrent “sinus headaches.” At the time of one such headache, she topically applied to the entire forehead and, with her eyes closed, to the sites overlying the frontal and maxillary sinuses a composition containing 1% (w/v) of N-Ac-Tyr-Pro-Phe-Phe-NHNH₂ (SEQ ID NO: 31) in WEP442. The headache completely subsided within 10 minutes.

The above result shows that endomorphin-2 tetrapeptide derivatives of the invention can be used to relieve sinus headache following topical administration.

### Example 56: Analgesic Effect on Finger Pain Caused by Thermal Burn

A female subject, age 61, suffered a thermal burn of her right fourth finger from touching a hot kitchen plate. A composition containing 1% (w/v) of N-Ac-Tyr-Pro-Phe-Phe-NHNH₂ (SEQ ID NO: 31) in WEP442 was applied topically to the finger within a few minutes, which completely relieved the pain almost instantly. The burned site did not developed a blister. Six days later, the burned site appeared normal.

The above result shows that endomorphin-2 tetrapeptide derivatives of the invention can be used to relieve pain from thermal burn following topical administration.

### Example 57: Analgesic Effect on Stress Pain in Muscles of the Neck

A 36 year old male farmer suffered substantial pain in the muscles of his neck the day after ploughing a field for several hours. During that time, he had almost constantly strained his neck muscles looking backward to monitor the performance of the plough behind. The pain was such that it disabled him from turning his head even slightly. Upon topical application of a composition containing 0.6% (w/v) of N-Pg-Tyr-Pro-Phe-Phe-NH₂ (SEQ ID NO: 44) in WEP442, the pain was almost completely relieved for about 4 hours. Reapplication of the solution at that time again relieved the pain for another 4 hours. With repeated applications, he was able to perform usual farm maintenance work for the entire day. After a night's rest, the pain subsided spontaneously.

The above result shows that endomorphin-2 tetrapeptide derivatives of the invention can be used to relieve neck pain following topical administration.

### Example 58: Analgesic Effect on Knee Pain Due to Injured Lateral Meniscus

A male subject, age 55, had knee pain at the site of the lateral meniscus of the right knee, secondary to repeated trauma in the course of his work as a house builder. He evaluated two (2) tetrapeptide derivatives for efficacy in relieving this pain, including N-Ac-Tyr-Pro-Phe-Phe-NH₂ (SEQ ID NO: 23) and N-Pg-Tyr-Pro-Phe-Phe-NH₂ (SEQ ID NO: 44), both as a 1% (w/v) solution composition in WEP442. One solution composition was topically applied during the course of a day; and the other solution composition was topically applied during the course of the following day. The evaluation continued over a period of 8 days in this schedule. Both solutions were found to perform equally well. Application of either solution composition to the affected knee provided complete relief of pain for about 4 hours. Re-application at the time of pain recurrence provided complete relief of pain for another 4 hour interval.

The above results show that endomorphin-2 tetrapeptide derivatives of the invention can be used to relieve knee pain following topical administration.

### Example 59: Analgesic Effect on Headache of Acupuncturist

A female acupuncturist, age 27, on a day while practicing her specialty, developed a severe headache that was not relieved by administering acupuncture procedures herself. She then topically applied to her forehead, periocular and temporal areas a composition containing 0.6% (w/v) of N-Pg-Tyr-Pro-Phe-Phe-NH₂ (SEQ ID NO: 44) in WEP442, which completely relieved her headache within approximately 2 minutes.

The above result shows that endomorphin-2 tetrapeptide derivatives of the invention can be used to relieve headache pain following topical administration.

### Example 60: Analgesic Effect on Persistent Headache Due to Inhalation of Gasoline Motor Exhaust Fumes

A male construction worker, age 33, suffered a severe nauseating headache on a day when, for about an hour, he was exposed to exhaust fumes from a gasoline motor driven dust blower early in the day. The headache persisted all day and into the evening. Topical application of a composition containing 0.5% (w/v) of N-Ac-Tyr-Pro-Phe-Phe-NH₂ (SEQ ID NO: 23) in WEP442, to almost the entire head, excluding the occipital scalp, completely relieved the headache within 2 minutes.

The above result shows that endomorphin-2 tetrapeptide derivatives of the invention can be used to relieve pain following topical administration.

### Example 61: Comparative Analgesic Efficacy in Psoriatic Arthritis of Wrists and Fingers

A female subject, age 54, with severe generalized psoriasis compared the topical efficacy of solution compositions of nine (9) tetrapeptide derivatives on arthritic pain of her wrists and fingers. All of the solution compositions contained 1% (w/v) of tetrapeptide derivative in WEP442.

The following endomorphin-2 and tetrapeptide derivatives thereof provided 4+ efficacy as judged by clinical evaluation: Tyr-Pro-Phe-Phe-NH₂ (SEQ ID NO: 1); N-Ac-Tyr-Pro-Phe-Phe-NH₂ (SEQ ID NO: 23); N-Pa-Tyr-Pro-Phe-Phe-NH₂ (SEQ ID NO: 64); N-Pg-Tyr-Pro-Phe-Phe-NH₂ (SEQ ID NO: 44); and N-Ac-Tyr-Pro-Phe-Phe-NHNH₂ (SEQ ID NO: 31).

The following endomorphin-1 and tetrapeptide derivatives thereof provided partial relief of 1+ efficacy as judged by clinical evaluation: Tyr-Pro-Trp-Phe-NH₂ (SEQ ID NO: 2); N-Ac-Tyr-Pro-Trp-Phe-NH₂ (SEQ ID NO: 272); and N-Pg-Tyr-Pro-Trp-Phe-NH₂ (SEQ ID NO: 274).

The following tetrapeptide derivative in ester form did not provide any pain relief, i.e., 0 efficacy as judged by clinical evaluation: N-Ac-Tyr-Pro-Phe-Phe-OEt (SEQ ID NO: 275).

The above result shows that endomorphin-2 and certain tested tetrapeptide derivatives thereof of the invention provided relief of arthritic pain following topical administration, whereas endomorphin-1 and tetrapeptide derivatives thereof provided minimal to no relief of arthritic pain.

### Example 62: Analgesic Efficacy of Psoriatic Arthritis

A female subject, age 54, with plague psoriasis since age 15 had painful psoriatic arthritis in the joints of both feet for the past 10-12 years. Topical application of a composition containing 1% (w/v) of N-Ac-Tyr-Pro-Phe-Phe-NH₂ (SEQ ID NO: 23) in WEP442 relieved the pain so that it was undetectable for 6-8 hours. Daily topical applications of the formulation for 5 days provided the same relief.

The above result shows that endomorphin-2 tetrapeptide derivatives of the invention can be used to relieve pain of psoriatic arthritic joints following topical administration.

### Example 63: Analgesic Effect on Wrist and Hand Pain

A 33 year old male hairdresser, after several hours of styling hair, experienced substantial discomfort on his hands that interfered with further pursuit of his work. Topical application of a composition containing 0.5% (w/v) of N-Pg-Tyr-Pro-Phe-Phe-NH₂ (SEQ ID NO: 44) in WEP442 to his hands provided complete relief, which allowed him to continue on with hair styling for several more clients.

The above result shows that endomorphin-2 tetrapeptide derivatives of the invention can be used to relieve hand pain following topical administration.

### Example 64: Analgesic Effect on Pharyngitis (“Sore Throat”)

A 94 year old male subject developed a respiratory tract infection, symptoms of which included coughing and pharyngitis. Swallowing of saliva was very painful for the subject. Clinical examination revealed intensely red inflammation of the posterior pharyngeal mucosa. A solution containing 0.5% (w/v) of N-Pg-Tyr-Pro-Phe-Phe-NH₂ (SEQ ID NO: 44) in a mixture of propylene glycol (10 parts by volume) and glycerol (90 parts by volume) was prepared. One teaspoonful of the solution was held in the posterior part of the mouth for about one minute and then swallowed. Within about 10 minutes, swallowing of liquids and semi-solid food could be done without pharyngeal pain. Such relief of painful swallowing lasted for about 3 hours. Repeated administrations provided the same results.

The above result shows that endomorphin-2 tetrapeptide derivatives of the invention can be used to relieve pharyngitis pain following oral administration.

Example 65: Analgesic Effect on Pain From Bulging Sub-Patellar Meniscus and Headache

A 64 year old male landscape worker slipped on the grass and twisted his right knee, which became too painful for him to walk on without physical support. Topical application to the knee of a composition containing 0.6% (w/v) N-Pg-Tyr-Pro-Phe-Phe-NH₂(SEQ ID NO: 44) in WEP442 provided complete relief of pain within 3-4 minutes. Relief lasted throughout the working day, which allowed him to perform non-stressful duties on the job. MM testing revealed a bulging meniscus under the medial side of the patella. Surgical corrective procedures were scheduled for 2 weeks later. In the meantime, topical applications of the above solution as needed allowed him to move about with no discomfort.

Several days after the incident causing the injury, he developed a bothersome headache, concentrated to the temporal areas. Topical application of the above solution provided almost immediate, complete relief of the headache.

The above results show that endomorphin-2 tetrapeptide derivatives of the invention can be used to relieve knee pain and headache following topical administration.

### Example 66: Analgesic Effect on Psoriatic Knee Pain

A 29 year old woman with plaque psoriasis of the scalp, elbows and knees for about 10 years had pain in her knees for the past 4-5 years. The knee pain had gradually worsened. Topical applications of a composition containing 0.5% (w/v) of N-Ac-Tyr-Pro-Phe-Phe-NH₂ (SEQ ID NO: 23) in WEP442 relieved her knee pain for about 6 hours, thus permitting her to pursue recreational hiking. After hiking for several hours, the knee pain sometimes re-occurred, but it was immediately relieved by topical application of the above solution composition.

The above result shows that endomorphin-2 tetrapeptide derivatives of the invention can be used to relieve severe psoriatic knee pain following topical administration.

### Example 67: Analgesic Effect on Pain in Feet and Neck with Plaque Psoriasis

A 59 year old woman with plaque psoriasis since her early teenage years had chronic foot pain for the past 10-15 years and neck pain for the past several years. The latter had been associated with, and aggravated by occupationally keeping her neck to one side as she held a phone against her head. Topical applications of a composition containing 0.5% (w/v) of N-Pg-Tyr-Pro-Phe-Phe-NH₂ (SEQ ID NO: 44) in WEP442 to her feet and neck provided almost complete relief of pain for 8-10 hours. Such topical applications provided relief for over one month when used.

The above result shows that endomorphin-2 tetrapeptide derivatives of the invention can be used to relieve neck and feet pain following topical administration.

### Example 68: Analgesic Effect on “Tennis Elbow” Pain

A 60 year old woman who played tennis recreationally for several decades developed increasingly severe pain in the elbow of her right arm. Two acupuncture treatments provided partial relief. Topical applications of a composition containing 0.5% (w/v) of N-Ac-Tyr-Pro-Phe-Phe-NH₂ (SEQ ID NO: 23) in WEP442 provided substantial relief of pain within 2-4 minutes of the topical application. After about 1 week of several applications daily, her range of arm motion returned to normal. She now plays tennis with no pain.

The above result shows that endomorphin-2 tetrapeptide derivatives of the invention can be used to relieve pain from tennis elbow following topical administration.

### Example 69: Analgesic Effect on Repeating Stress Headaches

A 29 year old woman had repeated stress headaches for several months. Such headaches had been relieved, or partially relieved, by over-the-counter medication. Over a 3 week interval, she had 5-6 such headaches, which had been completely relieved almost immediately by topical applications to the forehead, periorbital and temporal areas of a composition containing 0.5% (w/v) of N-Pg-Tyr-Pro-Phe-Phe-NH₂ (SEQ ID NO: 44) in WEP442.

The above result shows that endomorphin-2 tetrapeptide derivatives of the invention can relieve pain from stress headaches following topical administration.

### Example 70: Analgesic Effect on Post-Operative Tenderness and Soreness of Knee

A 64 year old male underwent surgical repair of a bulging meniscus of the right knee, after which there was substantial tenderness and soreness, particularly so upon his walking, which he was advised to do a few hours daily during the recovery period. Skin glue had been used for wound closure, which allowed for topical applications of a composition containing 0.6% (w/v) of N-Pg-Tyr-Pro-Phe-Phe-NH₂ (SEQ ID NO: 44) in WEP442 to the entire knee including 5 surgical wound sites. Each topical application gave rapid, almost complete, relief of pain sensations lasting 2-3 hours. Repeated topical applications provided freedom of movement throughout the day with negligible discomfort.

The above result show that endomorphin-2 tetrapeptide derivatives of the invention can be used to relieve pain of surgical wound sites following topical administration.

### Example 71: Analgesic Effect on Acute Pain Due to Foot Sprain and Knee Pain Following Patella (Knee Cap) Fracture

A 79 year old female subject suffered severe pain in her left knee and in the arch of her left foot following a forward fall to a wooden floor. Diagnoses of multiple fractures of the patella and a bruised foot were made by x-ray. Surgical repair of the knee was done, following which severe knee pain and acute pain in the arch of the foot persisted. Topical applications to the knee and foot of a composition containing 0.5% (w/v) of N-Pg-Tyr-Pro-Phe-Phe-NH₂ (SEQ ID NO: 44) in WEP442 provided immediate and complete relief of knee pain and foot pain for more than 6 hours. Topical applications 3-4 times daily allowed the subject to pursue rehabilitation procedures pain free.

The above result shows that endomorphin-2 tetrapeptide derivatives of the invention can be used to relieve knee and foot pain following topical administration.

### Example 72: Analgesic Effect on Migraine Headache

A female subject, age 55, who suffered from migraine headaches, woke up one morning with a headache, the type of which was uncertain. She topically applied to her forehead, periocular and temporal areas a composition containing 0.5% (w/v) of N-Pg-Tyr-Pro-Phe-Phe-NH₂ (SEQ ID NO: 44) in WEP442. The headache was promptly relieved and did not return.

The above result shows that endomorphin-2 tetrapeptide derivatives of the invention can be used to relieve headache pain following topical administration.

### Example 73: Analgesic Effect on Psoriatic Arthritis of Hands, Fingers and Ankles

A 55 year old male subject had psoriatic arthritis of the hands and finger joints and ankle joints for 4 years. Topical applications of a composition containing 0.5% (w/v) of N-Ac-Tyr-Pro-Phe-Phe-NH₂ (SEQ ID NO: 23) in WEP442 provided almost complete relief of pain for approximately 6 hours. Repeated topical applications provided comparable relief.

The above result shows that endomorphin-2 tetrapeptide derivatives of the invention can be used to relieve pain of psoriatic arthritis following topical administration.

### Example 74: Analgesic Effect on Severe Hangover Headache

A female subject, age 64, who had consumed excessive amounts of wine at an evening party, had a very substantial headache the next morning. She was provided a solution composition containing 0.5% (w/v) of N-Ac-Tyr-Pro-Phe-Phe-NH₂ (SEQ ID NO: 23) in WEP442 which she topically applied to the neck, peripheral scalp, and entire face and forehead. There was almost immediate, complete, relief of her aching head.

The above result shows that endomorphin-2 tetrapeptide derivatives of the invention can be used to relieve pain from hangover headache following topical administration.

### Example 75: Treatment of Restless Leg Syndrome

Restless leg syndrome (RLS) is a disorder in which involuntary movements of the legs occur during the sleeping hours. A male subject, age 60, presented with RLS for a duration of about 4 years. The disorder in the subject had not responded to therapy with opiates, nor to a benzodiazepine medication. He reported that topical application of a composition containing 1% (w/v) of N-Pg-Tyr-Pro-Phe-Phe-NH₂ (SEQ ID NO: 44) in WEP442 to his legs after an episode of leg movement had awakened him, completely prevented further involuntary leg movement for the rest of his sleeping hours.

The above result shows that endomorphin-2 tetrapeptide derivatives of the invention improved restless leg syndrome following topical administration.

### Example 76: Analgesic Effect on Arthritis Pain of the Hands

A male subject, age 72 and a veteran guitar player, was able to play his instrument in previous years for about an hour in group session performances. His capability in playing time gradually reduced to about 20 minutes due to painful arthritis of his hands, which had gradually worsened over the past decade. The subject reported that topical application to his hands of a composition containing 0.5% (w/v) of N-Pg-Tyr-Pro-Phe-Phe-NH₂ (SEQ ID NO: 44) in WEP442 enabled him to play his guitar for 1 hour pain free. He also noted that the quality of his performance had improved.

The above result shows that endomorphin-2 tetrapeptide derivatives of the invention can be used to improve arthritic pain in the hands following topical administration.

### Example 77: Analgesic Effect on Pain From Shoulder Strain

A female subject, age 83, presented with right shoulder pain for a duration of 5 days, which developed after carrying a very heavy grocery bag for several city blocks. The pain was increased by her attempts to lift her arm to an elevated position, and further increased from rotational arm movements. Topical application to the shoulder area of a composition containing 0.5% (w/v) of N-Pg-Tyr-Pro-Phe-Phe-NH₂ (SEQ ID NO: 44) in WEP442 gave rapid, complete relief of pain, so that any shoulder movement was pain free. Such relief lasted for about 6 hours, at which time re-application provided the same relief. Over the course of 3 days of repeated such applications, the pain did not recur.

The above result shows that endomorphin-2 tetrapeptide derivatives of the invention applied topically can be used to relieve pain and provide adequate treatment of disabling shoulder strain.

### Example 78: Analgesic Effect on Carpal Tunnel Syndrome

A male subject, age 29 and a computer operator, presented with symptoms of carpal tunnel syndrome, i.e. wrist pain and tingling sensations of the palm and fingers. Topical applications to the hand and fingers of a composition containing 0.5% (w/v) of N-Pg-Tyr-Pro-Phe-Phe-NH₂ (SEQ ID NO: 44) in WEP442 several times during working hours provided substantial relief of the symptoms. Relief occurred within 3 minutes of topical application and lasted for 2-4 hours.

The above result shows that topical application of endomorphin-2 tetrapeptide derivatives of the invention can be used to improve disabling symptoms of carpal tunnel syndrome.

### Example 79: Analgesic Effect on Psoriatic Arthritis

A female subject, age 59, presented with psoriatic arthritis of the fingers that was particularly painful in the right index finger joint and proximal joint of the left thumb. Topical applications of a composition containing 0.5% (w/v) of N-Ac-Tyr-Pro-Phe-Phe-NH₂ (SEQ ID NO: 23) in WEP442 to hands and fingers once daily at the start of working hours provided complete relief of pain throughout the day for 8 hours of office work.

The above result shows that topical application of endomorphin-2 tetrapeptide derivatives of the invention can be used to treat psoriatic arthritis.

### Example 80: Analgesic Effect on Pain and Nausea From Migraine Headache

A female subject, age 55, with a history of recurrent attacks of migraine, was provided a solution composition containing 0.5% (w/v) of N-Pg-Tyr-Pro-Phe-Phe-NH₂ (SEQ ID NO: 44) in WEP442 and a solution composition containing 0.5% (w/v) of N-Ac-Tyr-Pro-Phe-Phe-NHNH₂ (SEQ ID NO: 31) in WEP442. Each solution, when topically applied at the onset of symptoms, provided complete relief of pain within 5 minutes of topical application. Sensations of nausea that might have lingered went away after several minutes following a second topical application.

The above results show that topical application of endomorphin-2 tetrapeptide derivatives of the invention can be used to treat migraine headache and symptoms thereof, such as nausea.

### Example 81: Analgesic Effect on Pain From Trapezius Muscle Strain

A female subject, age 55, with persistent pain localized to a site medial to the upper right scapula (shoulder blade) topically applied to that site a composition containing 0.7% (w/v) of N-Ac-Tyr-Pro-Phe-Phe-NHCH₃(SEQ ID NO: 24) in propylene glycol: triethyl citrate : water (4:4:2 ratio). Complete relief of pain occurred within 2 minutes and lasted for about 4 hours.

This result shows that topical administration of endomorphin-2 tetrapeptide derivatives of the invention can be used to relieve pain from trapezius muscle strain.

### Example 82: Analgesic Effect on Pain and Improved Flexibility of Fingers and Wrists Affected by Osteoarthritis

A female subject, age 57, had osteoarthritis of the fingers and wrists for about 10 years, with substantial worsening over the past several months. Symptoms included painful finger joints and wrists. The pain was much worsened by flexion, so that making a first was too painful to try. Moreover, complete flexion had not been possible because of actual physical limitations. Anatomical signs of her disorder, e.g., enlarged finger joints, were conspicuous in her case. One topical application to the fingers and wrists of a composition containing 0.8% (w/v) of N-Ac-Tyr-Pro-Phe-Phe-NH₂ (SEQ ID NO: 23) in WEP442 provided a degree of improvement of pain within 20 minutes. A second topical application provided almost complete relief within another 20 minutes. After 1 week of repeated topical applications, it was found that complete relief of pain was continually sustained by one topical application every 12 hours, e.g., at 7 a.m. and 7 p.m., with no other topical applications in between. Additionally, complete flexion of the fingers and making of a tight first could be done without any discomfort.

These results show that endomorphin-2 tetrapeptide derivatives of the can be used to improve osteoarthritic fingers and wrists.

### Example 83: Analgesic Effect on Osteoarthritic Pain

A female subject, age 57, had osteoarthritis of the fingers and wrists for about 10 years, with substantial worsening over the past several months. Symptoms included painful finger joints and wrists. The pain was much worsened by flexion, so that making a first was too painful to try. Moreover, complete flexion had not been possible because of actual physical limitations. Anatomical signs of her disorder, e.g., enlarged finger joints, were conspicuous in her case. After 36 hours of no topical or systemic use of any analgesic agent, pain in the fingers and wrists was again present and substantial, so much so that she was unable to slice vegetables with a knife. Topical application of a solution composition containing 0.8% (w/v) of N-Ac-Tyr-Pro-Phe-Phe-NHCH₃ (SEQ ID NO: 24) in WEP442 to the hands, fingers and wrists completely relieved all pain within 5-7 minutes, and lasted for about 17 hours.

The above result shows that endomorphin-2 tetrapeptide derivatives of the invention can be used to improve osteoarthritic fingers and wrists.

### Example 84: Analgesic Effect on Pain From Psoriatic Arthritis

A female subject age, 57, had extensive plaque psoriasis and painful arthritis of the interphalangeal joints of both index fingers, left thumb and left 5^{th} finger, which compromised her work performance as an esthetician. The subject topically applied a solution composition containing 0.8% (w/v) of N-Ac-Tyr-Pro-Phe-Phe-NHCH₃ (SEQ ID NO: 24) in WEP442 to her painful fingers. Pain in the affected joints was completely relieved within 3-4 minutes, lasting for about 7 hours.

The above result shows that endomorphin-2 tetrapeptide derivatives of the invention can be used to improve psoriatic arthritis

### Example 85: Analgesic Effect on Lower Back Pain

A male subject, age 94, had lower back (lumbar) pain associated with sitting in a crouched position at a low work table for about 3 hours. Upon his sitting up straight, the pain became more pronounced and persisted as he walked around for 15-20 minutes. Generous topical application of a solution composition containing 0/8% (w/v) of N-Ac-Tyr-Pro-Phe-Phe-NHCH₃ (SEQ ID NO: 24) in WEP442 to his lower back skin resulted in complete resolution of all pain in the lower back within 5 minutes. The pain did not return.

The above result shows that endomorphin-2 tetrapeptide derivatives of the invention can be used to improve or eradicate lower back pain.

### Example 86: Analgesic Effect on Sunburn Pain

A female subject, age 48, experienced post-sunburn pain at the frontal hairline of the scalp, an area not earlier covered by sunscreen lotion at time of sun exposure several days earlier. She vigorously rubbed the area later to remove lingering skin scales, which caused irritation and pain. Topical application of a solution composition containing 0.8% (w/v) of N-Ac-Tyr-Pro-Phe-Phe-NHCH₃ (SEQ ID NO: 24) in WEP442 to the involved area provided almost immediate relief of the painful discomfort. Relief lasted for 4 hours. Several topical applications over the next 24 hours provided complete relief of pain. The condition was then resolved with the use of a hydrating skin cream.

The above result shows that endomorphin-2 tetrapeptide derivatives of the invention can be used to improve sunburn pain.

### Example 87: Pain Relief at Multiple Sites in One Subject by Multiple Endomorphin-2 Tetrapeptide Derivatives

A female caregiver subject, age 62, presented with (a) painful periostitis of the right upper tibia for about 3 months, (b) painful early osteoarthritis of the left knee for about 1 year, (c) painful left hip joint of about 6 months, and (d) lower back pain near the end of the work day for about 2 years.

Over the course of 16 days, this subject evaluated the analgesic efficacy of eight (8) endomorphin-2 tetrapeptide derivatives in WEP442 at a concentration of 0.8% (w/v) applied topically to the sites of pain. The degree of analgesic effect was graded on a scale of 0 to 4+, wherein 4+ represented complete relief of pain and zero represented no relief.

For the above subject, the composition containing 0.8% (w/v) of N-Ac-Tyr-Pro-Phe-Phe-NHCH₂CH₃ (SEQ ID NO: 25) in WEP442 provided complete relief of pain at all sites within 3-4 minutes, lasting for 5 hours or more (4+ effect). The lower back pain returned toward the end of the following work day.

For the above same subject, the composition containing 0.8% (w/v) of N-Ac-Tyr-Pro-Phe-Phe-NHCH₂C₆H₅ (SEQ ID NO: 29) in WEP442 also provided complete relief of pain at all sites within 3-4 minutes, lasting for 5 hours or more (4+ effect).

For the above same subject, the composition containing 0.8% (w/v) of N-Ac-Tyr-Pro-Phe-Phe-NHCH₂CH₂C₆H₅ (SEQ ID NO: 30) in WEP442 resulted in no detectable analgesic effect at any site (0 effect).

For the above same subject, the composition containing 0.8% (w/v) of N-Ac-Tyr-Pro-Phe-Phe-NHOH (SEQ ID NO: 43) in WEP442 also provided complete relief of pain at all sites within 1 minute, and lasted for 12 hours or more. Relief of lower back pain lasted for 20-24 hours (4+ effect).

For the above same subject, at the time of testing, a bunion on the right big toe was also painful. The composition containing 0.8% (w/v) of N-Ac-Tyr-Pro-Phe-Phe-NHNHAc (SEQ ID NO: 38) in WEP442 also provided complete relief of pain at all sites within 1-2 minutes, and lasted for 8-24 hours (4+ effect).

For the above same subject, the composition containing 0.8% (w/v) of N-Ac-Tyr-Pro-Phe-Phe-NHCH(CH₃)₂ (SEQ ID NO: 27) in WEP442 was topically applied to the right upper tibia, left knee and left hip. Complete relief of pain occurred within 3-4 minutes and lasted for at least 4 hours; and relief of pain of periostitis of the right upper tibia lasted for 7 hours (4+ effect).

For the above same subject, the composition containing 0.8% (w/v) of N-Fo-Tyr-Pro-Phe-Phe-NH₂ (SEQ ID NO: 104) in WEP442 was topically applied to 3 sites. Relief of pain was variable. On the left hip, relief was slow to occur and incomplete, i.e. in about 5 minutes, and only about 50% (2+). No relief of pain in the left knee was detected. Relief of pain in the right upper tibia was complete in about 4 minutes, lasting for 12 hours.

For the above same subject, the composition containing 0.8% (w/v) of N-Bz-Tyr-Pro-Phe-Phe-NH₂ (SEQ ID NO: 84) in WEP442 provided relief of pain within 2-4 minutes. On the left hip, the relief was complete, lasting for about 12 hours (4+ effect). On the left knee, relief was incomplete, about 50%, lasting for only about 3 hours (2+ effect). On the right upper tibia, relief was complete and lasted for about 12 hours (4+ effect). Lower lumbar pain responded completely, lasting for about 4 hours (4+ effect).

### Example 88: Analgesic Efficacy of Eight (8) Endomorphin-2 Tetrapeptide Derivatives on Severe Osteoarthritis

A male subject, age 94, with severe osteoarthritis of both knees for a duration of about 7 years evaluated numerous tetrapeptide derivatives over the past years. Several derivatives were found to substantially alleviate pain when applied topically. Over the course of 3 weeks, this subject evaluated the analgesic efficacy of eight (8) endomorphin-2 tetrapeptide derivatives in WEP442 at a concentration of 1% (w/v) applied topically to the knees.

For the above subject, the composition containing 1% (w/v) of N-Ac-Tyr-Pro-Phe-Phe-NHCH₂CH₃ (SEQ ID NO: 25) in WEP442 was topically applied to both knees and provided very good relief of pain within 3-5 minutes that lasted for 3-4 hours. Reapplication at the end of that time again provided the same effect. Application at bedtime provided relief of pain for 7 hours. The same results were achieved the next day (3+ to 4+ effect).

For the above same subject, the composition containing 1% (w/v) of N-Ac-Tyr-Pro-Phe-Phe-NHCH₂C₆H₅ (SEQ ID NO: 29) in WEP442 provided little relief of pain (1+ effect) with one application. A second application, about 15 minutes later, provided somewhat more relief (2+ effect).

For the above same subject, the composition containing 1% (w/v) of N-Ac-Tyr-Pro-Phe-Phe-NHCH₂CH₂C₆H₅ (SEQ ID NO: 30) in WEP442 had no detectable analgesic effect (0 effect).

For the above same subject, the composition containing 1% (w/v) of N-Ac-Tyr-Pro-Phe-Phe-NHOH (SEQ ID NO: 43) in WEP442 provided very good relief of pain within 3-4 minutes that lasted for about 5 hours. Application at 9:30 p.m. provided relief until 5:30 a.m., and similar results were achieved the next day (3+ to 4+ effect).

For the above same subject, the composition containing 1% (w/v) of N-Ac-Tyr-Pro-Phe-Phe-NHNHAc (SEQ ID NO: 38) in WEP442 also provided substantial relief of pain occurring in knee within 2-3 minutes of application to the left knee only. No modification of pain in the right knee, which had received application of vehicle WEP442 as a control, was detected. Application of the composition containing the tetrapeptide derivative to the right knee about 15 minutes later provided prompt relief of pain. Pain relief in both knees lasted for about 5 hours (3+ to 4+ effect).

For the above same subject, the composition containing 1% (w/v) of N-Ac-Tyr-Pro-Phe-Phe-NHCH(CH₃)₂ (SEQ ID NO: 27) in WEP442 topically applied to both knees provided moderate relief of pain within about 15 minutes. Relief lasted for about 3 hours. Re-application at that time provided the same degree of pain relief in about 5 minutes, which lasted about another 3 hours (2+ effect).

For the above same subject, the composition containing 1% (w/v) of N-Fo-Tyr-Pro-Phe-Phe-NH₂ (SEQ ID NO: 104) in WEP442 was applied to the left knee, and the composition containing 1% (w/v) of N-Bz-Tyr-Pro-Phe-Phe-NH₂ (SEQ ID NO: 84) in WEP442 was applied to the right knee moments later. Relief of pain in both knees seemed equivalent, was detectable in 5-7 minutes and lasted for about 4 hours. This procedure was repeated the next day with about the same results. The degree of pain relief from both solutions was rated as 3+ effect.

The above results indicate that endomorphin-2 tetrapeptide derivatives of the invention can provide significant relief of pain in topical treatment of very severe osteoarthritis.

### Example 89: Analgesic Effect on Hip Pain

A male subject, age 85, had severe lumber stenosis (lower back arthritis) for about 3 years caused by a fall from a ladder 7 years earlier. The subject had been pain free from lower back arthritis due to weekly acupuncture treatment, usually at 10 AM every Tuesday. On one Friday evening, the subject suddenly developed severe pain on the left hip and the pain persisted. The subject topically applied a composition containing 1% (w/v) of N-Ac-Tyr-Pro-Phe-Phe-NH₂ (SEQ ID NO: 23) in WEP442 solution to the left hip skin area, and the pain disappeared in about 2 minutes. The hip pain did not return even after 12 hours (4+ effect).

The above result shows that endomorphin-2 tetrapeptide derivatives of the invention can provide prompt relief of pain from arthritis on topical application.

### Example 90: Analgesic Effect on Post-Surgery Pain and Discomfort

A female subject, age 62, had painful discomfort of her back neck for 2 years caused by a surgical titanium implant of the 7^{th} cervical vertebra 2 years earlier. During the past years, the pain and discomfort had been improved from occasional treatments with acupuncture and massage therapy. However, such treatments provided only short term relief of pain and discomfort, usually one or two pain free days after each treatment.

On one afternoon, pain and discomfort suddenly developed. The subject topically applied a composition containing 1% (w/v) of N-Ac-Tyr-Pro-Phe-Phe-NH₂ (SEQ ID NO: 23) in WEP442 solution to skin of her back neck area. The subject felt a slight numbing effect within a few minutes, and the pain and discomfort disappeared in about 5 minutes. The subject was free of pain and discomfort for at least 12 hours (4+ effect).

The above result shows that endomorphin-2 tetrapeptide derivatives of the invention can provide prompt relief of pain and discomfort from post-surgery implantation.

### Example 91: Absence of Analgesic Effect on Experimentally Induced Skin Pain

A male subject, age 94, rolled onto his left forearm with forceful pressure a roller having many tiny stainless steel needles 1 mm in length, which penetrated through the epidermis causing sharp pain as well as punctate bleeding. Topical application of a composition containing 1% (w/v) of N-Ac-Tyr-Pro-Phe-Phe-NH₂ (SEQ ID NO: 23) in WEP442 before and after such mechanically induced wounding did not prevent nor relieve the sharp punctate pain.

The same subject above self-injected into his left forearm a sterile aqueous solution (pH 6.2) containing 0.1% of Tyr-Pro-Phe-Phe-NH₂ (SEQ ID NO: 1), intradermally (0.1 ml) and subcutaneously (0.2 ml). Such injections did not prevent pain caused by needling or pinching of the injected sites.

The above results show that endomorphin-2 tetrapeptide derivatives did not provide any relief of pain from mechanically induced injury to the skin.

### Example 92: Influence of Vehicle on Analgesic Efficacy

A male subject, age 94, with osteoarthritis of the knees, which progressively had become more painful over the years, noticed that the pain now extended to involve the lower legs. Earlier, he had found that application of N-Ac-Tyr-Pro-Phe-Phe-NH₂ (SEQ ID NO: 23) dissolved in WEP442 to the knees provided substantial relief of pain.

The following procedures were carried out to determine if modification of liquid vehicles could influence analgesic performance. In all three (3) preparations, the tetrapeptide derivative used was N-Ac-Tyr-Pro-Phe-Phe-NH₂ (SEQ ID NO: 23) at a concentration of 4% (w/v).

Preparations:

Preparation A: in vehicle; WEP442

Preparation B: in vehicle; propylene glycol 50, triethyl citrate 50 (v/v)

Preparation C: in vehicle; water 20, ethanol 10, propylene glycol 40, triethyl citrate 30 (v/v)

Procedure
Day 1: left knee, Preparation A applied at 10 AM and 3 PM right knee, Preparation B applied at 10 AM and 3 PM
Day 2: repeat same as Day 1
Day 3: left knee, Preparation C applied at 10 AM and 3 PM right knee, Preparation A applied at 10 AM and 3 PM
Day 4: repeat same as on Day 3

Pain relief with use of Preparation A was moderate, beginning 2-3 minutes after application, and lasting for about 3 hours. Pain relief with the use of Preparation B was substantial, nearly complete, lasting for about 3 hours. Pain relief with the use of Preparation C was substantial, nearly complete and lasting for about 3 hours. In all instances, relief of pain extended from the knees to the lower legs.

The foregoing results indicate that pain relief provided by endomorphin-2 tetrapeptide derivatives of the invention is achieved by topical application, and that all three vehicles provided nearly the same analgesic effect.

### Example 93: Multiple Endomorphin-2 Related Tetrapeptide Derivatives Evaluated for Relief of Pain Associated With Psoriatic Arthritis

A female subject, age 57, with extensive plaque psoriasis and psoriatic arthritis of interphalangeal joints of the fingers evaluated multiple tetrapeptide derivatives for comparative effectiveness on relief of her painful fingers. Over the course of 4 weeks, this subject evaluated the analgesic efficacy of ten (10) endomorphin-2 tetrapeptide derivatives in WEP442 at a concentration of 0.8% (w/v) applied topically to both hands, which included all fingers. The degree of analgesic effectiveness was graded on scale of 0 to 4+, wherein 4+ represented complete relief of pain and zero represented no relief.

For this subject, the composition containing 0.8% (w/v) of N-Ac-Tyr-Pro-Phe-Phe-NHCH₂CH₃ (SEQ ID NO: 25) in WEP442 provided complete relief of pain of arthritis within 1-2 minutes, lasting for about 7 hours (4+ effect).

For the above same subject, the composition containing 0.8% (w/v) of N-Ac-Tyr-Pro-Phe-Phe-NHCH₂C₆H₅ (SEQ ID NO: 29) in WEP442 provided substantial relief of pain within 3-5 minutes, lasting for about 6 hours (3+ effect).

For the above same subject, the composition containing 0.8% (w/v) of N-Ac-Tyr-Pro-Phe-Phe-NHCH₂CH₂C₆H₅ (SEQ ID NO: 30) in WEP442 provided no detectable analgesic effect (0 effect).

For the above same subject, the composition containing 0.8% (w/v) of N-Ac-Ty-Pro-Phe-Phe-NHOH (SEQ ID NO: 43) in WEP442 provided complete relief of pain within 1-2 minutes, but the relief lasted only about 2 hours (2+ effect).

For the above same subject, the composition containing 0.8% (w/v) of N-Ac-Tyr-Pro-Phe-Phe-NHNHAc (SEQ ID NO: 38) in WEP442 provided complete relief of pain within 2 minutes, lasting for about 7 hours (4+ effect).

For the above same subject, the composition containing 0.8% (w/v) of N-Ac-Tyr-Pro-Phe-Phe NCH(CH₃)₂ (SEQ ID NO: 27) in WEP442 provided only minimal relief of pain over 5-6 minutes and lasted only 2-3 hours (1+ effect).

For the above same subject, the composition containing 0.8% (w/v) of N-Fo-Ty-Pro-Phe-Phe-NH₂ (SEQ ID NO: 104) in WEP442 likewise provided only minimal relief of pain, was slow in onset over 5-6 minutes, and lasted only 2-3 hours (1+ effect).

For the above same subject, the composition containing 0.8% (w/v) of N-Bz-Tyr-Pro-Phe-Phe NH₂ (SEQ ID NO: 84) in WEP442 provided incomplete, but substantial relief of pain, had a rather slow onset over 4-5 minutes, and lasted for about 10 hours (2+ effect).

For the above same subject, the composition containing 0.8% (w/v) of N-Pa-Tyr-Pro-Phe-Phe-NHNH₂ (SEQ ID NO: 71) in WEP442 provided complete relief of pain promptly within 2-3 minutes, lasting for about 7 hours (4+ effect).

For the above same subject, the composition containing 0.8% (w/v) N-Pa-Tyr-Pro-Phe-Phe-NHOH (SEQ ID NO: 83) in WEP442 provided complete relief of pain within 2-3 minutes, lasting for about 7 hours (4+ effect).

The foregoing results indicate that topical application of endomorphin-2 tetrapeptide derivatives of the invention can be used to treat psoriatic arthritis.

### Example 94: Analgesic Effect on Osteoarthritic Knees From Systemic Intra-Articular Injection

A male subject, age 94, with osteoarthritis of the knees that progressively worsened over the years, evaluated the analgesic effectiveness of many topical preparations of tetrapeptide derivatives of the invention on numerous occasion. After such experiences, he decided to self-inject his knees with a sterile aqueous preparation of N-Ac-Tyr-Pro-Phe-Phe-NH₂, (SEQ ID NO: 23) at a concentration of approximately 1% (w/v) in water. Via the sub-patellar route, each knee was injected with 1 ml of the preparation, containing approximately 10 mg of the tetrapeptide . Mild discomfort from the injection was felt, which was relieved by the subject lying down with legs elevated for about 20 minutes. Over the ensuing 24 hours, the knee pain gradually diminished to a level that while in a sitting position at a desk, the subject discerned virtually no pain. Walking about with the use of a wheeled walker at that time, the subject experienced knee pain, but at a level distinctly lower than earlier days before the injection. This degree of substantially diminished knee pain continued for an additional 16 hours when the return of pain was detected (3+ effect).

The foregoing result indicates that systemic administration of endomorphin-2 tetrapeptide derivatives of the invention is therapeutically effective for treatment of osteoarthritic pain.

### Example 94: Analgesic Effect on Osteoarthritic Knees From Systemic Intra-Articular Injection

A male subject, age 94, with osteoarthritis of the knees that progressively worsened over the years, evaluated the analgesic effectiveness of many topical preparations of tetrapeptide derivatives of the invention on numerous occasions. After such experiences he decided to self-inject his knees with a sterile aqueous preparation of N-Bz-Tyr-Pro-Phe-Phe-NHNH₂ (SEQ ID NO: 91) at a concentration of 0.08% (0.8 mg/ml) in water. Each knee was injected with 1 ml (0.8 mg) of the preparation via the sub-patellar route. There was no discomfort from the injections as compared with other injections. The pains in both knees gradually disappeared and the pain relief lasted for 11 hours. The knees felt more comfortable than usual after 24 hours. The improvement from systemic injection was judged to be more than 90% by clinical evaluation.

The foregoing result indicates that the endomorphin-2 tetrapeptide derivative of the invention is therapeutically effective for systemic treatment of osteoarthritic pain.

### Example 95: Comparative Analgesic Efficacy on Pain of Psoriatic Arthritis

A female subject, age 54, had severe generalized psoriasis, the onset of which began at about age of 16. Treatment of the subject over the years included methotrexate and other antimetabolites, and in recent years, drugs known as biologic medications. While these agents provided intervals of relief, none provided lasting benefit. Over the past decade, painful arthritis developed in both hands and knees, and in numerous joints of the fingers. Three related tetrapeptide derivatives, 1% (w/v) in vehicle WEP442, were topically applied to the fingers and wrists of both hands one at a time. Analgesic efficacy of each was judged to be as follows.
(A) N-Bz-Tyr-Pro-Phe-Phe-NHNH₂ (SEQ ID NO: 91) provided rapid complete relief of pain within 2-5 minutes, lasting for about 24 hours. Analgesic efficacy was rated by the subject as 100%.
(B) N-Bz-Tyr-Pro-Phe-Phe-NHOH (SEQ ID NO: 83) also provided rapid complete relief of pain within 2-5 minutes, lasting for about 10 hours. Analgesic efficacy was rated by subject as 90%.
(C) N-Ac-Tyr-Pro-Phe-Phe-NHCH₂CH₂CH₃ (SEQ ID NO: 26) provided slower relief of pain, more than 5 minutes, and lasted for less than 6 hours. Analgesic efficacy was rated by the subject as 75%.

The above results show that N-Bz-Tyr-Pro-Phe-Phe-NHNH₂ (SEQ ID NO: 91) of the invention was the most efficacious for relieving arthritic pain in psoriasis following topical administration.

In all the test cases as described in the above Examples, the control vehicles showed no detectable analgesic effect or zero improvement.

Pains and medical conditions or disorders improved or relieved by topical application of endomorphin-2 or a tetrapeptide derivative thereof of the invention based on the studies described herein can be summarized as follows:

| Conditions or Disorders | Number of Cases |
|---|---|
| Arthritis: | |
| Osteoarthritis: fingers, knees, shoulders, joints etc. | 21 |
| Psoriatic arthritis: fingers, wrists, ankles, joints etc. | 8 |
| Headache: | |
| Migraine | 5 |
| Non-migraine: ordinary, hangover etc. | 14 |
| Dental/toothache; gum etc. | 4 |
| Lipoma | 1 |
| Muscle Pain | 3 |
| Pharyngitis | 1 |
| Sprain/trauma: hands, feet, fingers, toes etc. | 18 |
| Sunburn/thermal burn: | 2 |
| Viral infection: herpes zoster, shingles | 1 |
| Wounds: post-operative, injection sites etc. | 3 |

In sum, the clinical test results described herein indicate that endomorphin-2 and tetrapeptide derivatives thereof of formula (I) according to the invention are therapeutically effective for treating pain and arthritis when topically or systemically administered to a subject, particularly when topically administered to a human subject.

We claim:

1. An endomorphin-2 tetrapeptide derivative of formula (I):
R₁-Tyr-Pro-Phe-Phe-R₂ formula (I)
or a pharmaceutically acceptable salt thereof, wherein R₁ is H or an acyl radical having up to 29 carbon atoms; R₂ is NHR₃ or NHNHR₄, or alternatively the carboxy terminus —COR₂ is CN; R₃ is H, OH, an alkyl, aralkyl or aryl radical having up to 9 carbon atoms; and R₄ is H, an alkyl, aralkyl, aryl or acyl radical having up to 9 carbon atoms; provided that when R₂ is NH₂, R₁ is not H or Ac.

2. The endomorphin-2 tetrapeptide derivative of formula (I) of claim 1, wherein R₁ is selected from the group consisting of H, Ab, Ac, Ba, Bo, Bz, Fo, Hd, He, Hp, Ip, Le, Ln, Na, Np, Oa, Pa, Pc, Pe and Pg; and R₂ is selected from the group consisting of NH₂, NHCH₃, NHCH₂CH₃, NHCH₂CH₂CH₃; NHCH(CH₃)₂; NHC₆H₅; NHCH₂C₆H₅; NHNH₂; NHNHCH₃; NHNHCH₂CH₃; NHNHCH₂CH₂CH₃; NHNHCH(CH₃)₂; NHNHC₆H₅; NHNHCH₂C₆H₅; NHNHAc; NHNHPa; NHNHBz; NHNHOa; NHNHFo; and NHOH.

3. The endomorphin-2 tetrapeptide derivative of formula (I) of claim 1, wherein the endomorphin-2 tetrapeptide derivative of formula (I) is selected from the group consisting of: N-Ab-Tyr-Pro-Phe-Phe-NH₂ (SEQ ID NO: 124); N-Ba-Tyr-Pro-Phe-Phe-NH₂(SEQ ID NO: 202); N-Bo-Tyr-Pro-Phe-Phe-NH₂ (SEQ ID NO: 144); N-Bz-Tyr-Pro-Phe-Phe-NH₂(SEQ ID NO: 84); N-Fo-Tyr-Pro-Phe-Phe-NH₂ (SEQ ID NO: 104); N-Hd-Tyr-Pro-Phe-Phe-NH₂(SEQ ID NO: 164); N-He-Tyr-Pro-Phe-Phe-NH₂ (SEQ ID NO: 204); N-Hp-Tyr-Pro-Phe-Phe-NH₂(SEQ ID NO: 205); N-Ip-Tyr-Pro-Phe-Phe-NH₂ (SEQ ID NO: 220); N-Le-Tyr-Pro-Phe-Phe-NH₂(SEQ ID NO: 221); N-Ln-Tyr-Pro-Phe-Phe-NH₂ (SEQ ID NO: 222); N-Na-Tyr-Pro-Phe-Phe-NH₂(SEQ ID NO: 207); N-Np-Tyr-Pro-Phe-Phe-NH₂ (SEQ ID NO: 223); N-Oa-Tyr-Pro-Phe-Phe-NH₂(SEQ ID NO: 205); N-Pa-Tyr-Pro-Phe-Phe-NH₂ (SEQ ID NO: 64); N-Pc-Tyr-Pro-Phe-Phe-NH₂(SEQ ID NO: 183); N-Pe-Tyr-Pro-Phe-Phe-NH₂ (SEQ ID NO: 203); N-Pg-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Ba-Tyr-Pro-Phe-Phe-NHNH₂ (SEQ ID NO: 208); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Fo-Tyr-Pro-Phe-Phe-NHNH₂ (SEQ ID NO: 111); N-Hd-Tyr-Pro-Phe-Phe-NHNH₂ (SEQ ID NO: 172); N-He-Tyr-Pro-Phe-Phe-NHNH₂ (SEQ ID NO: 210); N-Hp-Tyr-Pro-Phe-Phe-NHNH₂ (SEQ ID NO: 211); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂ (SEQ ID NO: 212); N-Pa-Tyr-Pro-Phe-Phe-NHNH₂ (SEQ ID NO: 71); N-Pc-Tyr-Pro-Phe-Phe-NHNH₂ (SEQ ID NO: 191); N-Pe-Tyr-Pro-Phe-Phe-NHNH₂ (SEQ ID NO: 209); N-Pg-Tyr-Pro-Phe-Phe-NHNH₂ (SEQ ID NO: 51); Tyr-Pro-Phe-Phe-NHNHAc (SEQ ID NO: 17); N-Ab-Tyr-Pro-Phe-Phe-NHNHAc (SEQ ID NO: 138); N-Ac-Tyr-Pro-Phe-Phe-NHNHAc (SEQ ID NO: 38); N-Ba-Tyr-Pro-Phe-Phe-NHNHAc (SEQ ID NO: 228); N-Bo-Tyr-Pro-Phe-Phe-NHNHAc (SEQ ID NO: 158); N-Bz-Tyr-Pro-Phe-Phe-NHNHAc (SEQ ID NO: 98); N-Fo-Tyr-Pro-Phe-Phe-NHNHAc (SEQ ID NO: 118); N-Hd-Tyr-Pro-Phe-Phe-NHNHAc (SEQ ID NO: 179); N-He-Tyr-Pro-Phe-Phe-NHNHAc (SEQ ID NO: 229); N-Hp-Tyr-Pro-Phe-Phe-NHNHAc (SEQ ID NO: 230); N-Ip-Tyr-Pro-Phe-Phe-NHNHAc (SEQ ID NO: 231); N-Le-Tyr-Pro-Phe-Phe-NHNHAc (SEQ ID NO: 232); N-Ln-Tyr-Pro-Phe-Phe-NHNHAc (SEQ ID NO: 233); N-Na-Tyr-Pro-Phe-Phe-NHNHAc (SEQ ID NO: 234); N-Np-Tyr-Pro-Phe-Phe-NHNHAc (SEQ ID NO: 235); N-Oa-Tyr-Pro-Phe-Phe-NHNHAc (SEQ ID NO: 236); N-Pa-Tyr-Pro-Phe-Phe-NHNHAc (SEQ ID NO: 78); N-Pc-Tyr-Pro-Phe-Phe-NHNHAc (SEQ ID NO: 198); N-Pe-Tyr-Pro-Phe-Phe-NHNHAc (SEQ ID NO: 237); N-Pg-Tyr-Pro-Phe-Phe-NHNHAc (SEQ ID NO: 198); Tyr-Pro-Phe-Phe-CN (SEQ ID NO: 238); N-Ab-Tyr-Pro-Phe-Phe-CN (SEQ ID NO: 239); N-Ac-Tyr-Pro-Phe-Phe-CN (SEQ ID NO: 240); N-Ba-Tyr-Pro-Phe-Phe-CN (SEQ ID NO: 241); N-Bo-Tyr-Pro-Phe-Phe-CN (SEQ ID NO: 242); N-Bz-Tyr-Pro-Phe-Phe-CN (SEQ ID NO: 243); N-Fo-Tyr-Pro-Phe-Phe-CN (SEQ ID NO: 244); N-Hd-Tyr-Pro-Phe-Phe-CN (SEQ ID NO: 245); N-He-Tyr-Pro-Phe-Phe-CN (SEQ ID NO: 246); N-Hp-Tyr-Pro-Phe-Phe-CN (SEQ ID NO: 247); N-Ip-Tyr-Pro-Phe-Phe-CN (SEQ ID NO: 248); N-Le-Tyr-Pro-Phe-Phe-CN (SEQ ID NO: 249); N-Ln-Tyr-Pro-Phe-Phe-CN (SEQ ID NO: 250); N-Na-Tyr-Pro-Phe-Phe-CN (SEQ ID NO: 251); N-Np-Tyr-Pro-Phe-Phe-CN (SEQ ID NO: 252); N-Oa-Tyr-Pro-Phe-Phe-CN (SEQ ID NO: 253); N-Pa-Tyr-Pro-Phe-Phe-CN (SEQ ID NO: 254); N-Pc-Tyr-Pro-Phe-Phe-CN (SEQ ID NO: 255); N-Pe-Tyr-Pro-Phe-Phe-CN (SEQ ID NO: 256); and N-Pg-Tyr-Pro-Phe-Phe-CN (SEQ ID NO: 257).

4. The endomorphin-2 tetrapeptide derivative of formula (I) of claim 1, wherein the endomorphin-2 tetrapeptide derivative of formula (I) is selected from the group consisting of: Tyr-Pro-Phe-Phe-NHNH₂ (SEQ ID NO: 10); N-Ab-Tyr-Pro-Phe-Phe-NH₂ (SEQ ID NO: 124); N-Ac-Tyr-Pro-Phe-Phe-NH₂ (SEQ ID NO: 23); N-Bo-Tyr-Pro-Phe-Phe-NH₂ (SEQ ID NO: 144); N-Bz-Tyr-Pro-Phe-Phe-NH₂ (SEQ ID NO: 84); N-Hd-Tyr-Pro-Phe-Phe-NH₂ (SEQ ID NO: 164); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Np-Tyr-Pro-Phe-Phe-NH₂ (SEQ ID NO: 223); N-Oa-Tyr-Pro-Phe-Phe-NH₂ (SEQ ID NO: 205); N-Pc-Tyr-Pro-Phe-Phe-NH₂ (SEQ ID NO: 183); N-Pg-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Hd-Tyr-Pro-Phe-Phe-NHNH₂ (SEQ ID NO: 172); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂(SEQ ID NO: 212); N-Pc-Tyr-Pro-Phe-Phe-NHNH₂ (SEQ ID NO: 191); and N-Pg-Tyr-Pro-Phe-Phe-NHNH₂ (SEQ ID NO: 51).

5. A composition comprising a therapeutically effective amount of the endomorphin-2 tetrapeptide derivative of formula (I) of claim 1 and a pharmaceutically or cosmetically acceptable carrier.

6. A composition for topical administration comprising at least 0.2% by weight or volume of endomorphin-2 or a tetrapeptide derivative thereof of formula (I):
Rᵢ-Tyr-Pro-Phe-Phe-R₂ formula (I)
or a pharmaceutically acceptable salt thereof, and optionally a pharmaceutically or cosmetically acceptable carrier, wherein R₁ is H or an acyl radical having up to 29 carbon atoms; R₂ is NHR₃ or NHNHR₄, or alternatively the carboxy terminus —COR₂ is CN; R₃ is H, OH, an alkyl, aralkyl or aryl radical having up to 9 carbon atoms; and R₄ is H, an alkyl, aralkyl, aryl or acyl radical having up to 9 carbon atoms.

7. The composition of claim 6, wherein R₁ is selected from the group consisting of H, Ab, Ac, Ba, Bo, Bz, Fo, Hd, He, Hp, Ip, Le, Ln, Na, Np, Oa, Pa, Pc, Pe and Pg; and R₂ is selected from the group consisting of NH₂, NHCH₃, NHCH₂CH₃, NHCH₂CH₂CH₃; NHCH(CH₃)₂; NHC₆H₅; NHCH₂C₆H₅; NHNH₂; NHNHCH₃; NHNHCH₂CH₃; NHNHCH₂CH₂CH₃; NHNHCH(CH₃)₂; NHNHC₆H₅; NHNHCH₂C₆H₅; NHNHAc; NHNHPa; NHNHBz; NHNHOa; NHNHFo; and NHOH.

8. The composition of claim 6, wherein endomorphin-2 or the tetrapeptide derivative thereof of formula (I) is selected from the group consisting of: Tyr-Pro-Phe-Phe-NH₂ (SEQ ID NO: 1); N-Ab-Tyr-Pro-Phe-Phe-NH₂ (SEQ ID NO: 124); N-Ac-Tyr-Pro-Phe-Phe-NH₂ (SEQ ID NO: 23); N-Ba-Tyr-Pro-Phe-Phe-NH₂ (SEQ ID NO: 202); N-Bo-Tyr-Pro-Phe-Phe-NH₂ (SEQ ID NO: 144); N-Bz-Tyr-Pro-Phe-Phe-NH₂ (SEQ ID NO: 84); N-Fo-Tyr-Pro-Phe-Phe-NH₂ (SEQ ID NO: 104); N-Hd-Tyr-Pro-Phe-Phe-NH₂ (SEQ ID NO: 164); N-He-Tyr-Pro-Phe-Phe-NH₂ (SEQ ID NO: 204); N-Hp-Tyr-Pro-Phe-Phe-NH₂ (SEQ ID NO: 205); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Na-Tyr-Pro-Phe-Phe-NH₂ (SEQ ID NO: 207); N-Np-Tyr-Pro-Phe-Phe-NH₂ (SEQ ID NO: 223); N-Oa-Tyr-Pro-Phe-Phe-NH₂ (SEQ ID NO: 205); N-Pa-Tyr-Pro-Phe-Phe-NH₂ (SEQ ID NO: 64); N-Pc-Tyr-Pro-Phe-Phe-NH₂ (SEQ ID NO: 183); N-Pe-Tyr-Pro-Phe-Phe-NH₂ (SEQ ID NO: 203); N-Pg-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Ba-Tyr-Pro-Phe-Phe-NHNH₂ (SEQ ID NO: 208); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Fo-Tyr-Pro-Phe-Phe-NHNH₂ (SEQ ID NO: 111); N-Hd-Tyr-Pro-Phe-Phe-NHNH₂ (SEQ ID NO: 172); N-He-Tyr-Pro-Phe-Phe-NHNH₂ (SEQ ID NO: 210); N-Hp-Tyr-Pro-Phe-Phe-NHNH₂ (SEQ ID NO: 211); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂ (SEQ ID NO: 212); N-Pa-Tyr-Pro-Phe-Phe-NHNH₂ (SEQ ID NO: 71); N-Pc-Tyr-Pro-Phe-Phe-NHNH₂ (SEQ ID NO: 191); N-Pe-Tyr-Pro-Phe-Phe-NHNH₂ (SEQ ID NO: 209); N-Pg-Tyr-Pro-Phe-Phe-NHNH₂ (SEQ ID NO: 51); Tyr-Pro-Phe-Phe-NHNHAc (SEQ ID NO: 17); N-Ab-Tyr-Pro-Phe-Phe-NHNHAc (SEQ ID NO: 138); N-Ac-Tyr-Pro-Phe-Phe-NHNHAc (SEQ ID NO: 38); N-Ba-Tyr-Pro-Phe-Phe-NHNHAc (SEQ ID NO: 228); N-Bo-Tyr-Pro-Phe-Phe-NHNHAc (SEQ ID NO: 158); N-Bz-Tyr-Pro-Phe-Phe-NHNHAc (SEQ ID NO: 98); N-Fo-Tyr-Pro-Phe-Phe-NHNHAc (SEQ ID NO: 118); N-Hd-Tyr-Pro-Phe-Phe-NHNHAc (SEQ ID NO: 179); N-He-Tyr-Pro-Phe-Phe-NHNHAc (SEQ ID NO: 229); N-Hp-Tyr-Pro-Phe-Phe-NHNHAc (SEQ ID NO: 230); N-Ip-Tyr-Pro-Phe-Phe-NHNHAc (SEQ ID NO: 231); N-Le-Tyr-Pro-Phe-Phe-NHNHAc (SEQ ID NO: 232); N-Ln-Tyr-Pro-Phe-Phe-NHNHAc (SEQ ID NO: 233); N-Na-Tyr-Pro-Phe-Phe-NHNHAc (SEQ ID NO: 234); N-Np-Tyr-Pro-Phe-Phe-NHNHAc (SEQ ID NO: 235); N-Oa-Tyr-Pro-Phe-Phe-NHNHAc (SEQ ID NO: 236); N-Pa-Tyr-Pro-Phe-Phe-NHNHAc (SEQ ID NO: 78); N-Pc-Tyr-Pro-Phe-Phe-NHNHAc (SEQ ID NO: 198); N-Pe-Tyr-Pro-Phe-Phe-NHNHAc (SEQ ID NO: 237); N-Pg-Tyr-Pro-Phe-Phe-NHNHAc (SEQ ID NO: 198); Tyr-Pro-Phe-Phe-CN (SEQ ID NO: 238); N-Ab-Tyr-Pro-Phe-Phe-CN (SEQ ID NO: 239); N-Ac-Tyr-Pro-Phe-Phe-CN (SEQ ID NO: 240); N-Ba-Tyr-Pro-Phe-Phe-CN (SEQ ID NO: 241); N-Bo-Tyr-Pro-Phe-Phe-CN (SEQ ID NO: 242); N-Bz-Tyr-Pro-Phe-Phe-CN (SEQ ID NO: 243); N-Fo-Tyr-Pro-Phe-Phe-CN (SEQ ID NO: 244); N-Hd-Tyr-Pro-Phe-Phe-CN (SEQ ID NO: 245); N-He-Tyr-Pro-Phe-Phe-CN (SEQ ID NO: 246); N-Hp-Tyr-Pro-Phe-Phe-CN (SEQ ID NO: 247); N-Ip-Tyr-Pro-Phe-Phe-CN (SEQ ID NO: 248); N-Le-Tyr-Pro-Phe-Phe-CN (SEQ ID NO: 249); N-Ln-Tyr-Pro-Phe-Phe-CN (SEQ ID NO: 250); N-Na-Tyr-Pro-Phe-Phe-CN (SEQ ID NO: 251); N-Np-Tyr-Pro-Phe-Phe-CN (SEQ ID NO: 252); N-Oa-Tyr-Pro-Phe-Phe-CN (SEQ ID NO: 253); N-Pa-Tyr-Pro-Phe-Phe-CN (SEQ ID NO: 254); N-Pc-Tyr-Pro-Phe-Phe-CN (SEQ ID NO: 255); N-Pe-Tyr-Pro-Phe-Phe-CN (SEQ ID NO: 256); and N-Pg-Tyr-Pro-Phe-Phe-CN (SEQ ID NO: 257).

9. The composition of claim 6, wherein endomorphin-2 or the tetrapeptide derivative thereof of formula (I) is selected from the group consisting of: N-Ab-Tyr-Pro-Phe-Phe-NH₂(SEQ ID NO: 124); N-Ac-Tyr-Pro-Phe-Phe-NH₂ (SEQ ID NO: 23); N-Bo-Tyr-Pro-Phe-Phe-NH₂ (SEQ ID NO: 144); N-Bz-Tyr-Pro-Phe-Phe-NH₂ (SEQ ID NO: 84); N-Hd-Tyr-Pro-Phe-Phe-NH₂ (SEQ ID NO: 164); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Np-Tyr-Pro-Phe-Phe-NH₂ (SEQ ID NO: 223); N-Oa-Tyr-Pro-Phe-Phe-NH₂(SEQ ID NO: 205); N-Pc-Tyr-Pro-Phe-Phe-NH₂ (SEQ ID NO: 183); N-Pg-Tyr-Pro-Phe-Phe-NH₂ (SEQ ID NO: 44); Tyr-Pro-Phe-Phe-NHNH₂(SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Hd-Tyr-Pro-Phe-Phe-NHNH₂ (SEQ ID NO: 172); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂(SEQ ID NO: 212); N-Pc-Tyr-Pro-Phe-Phe-NHNH₂ (SEQ ID NO: 191); and N-Pg-Tyr-Pro-Phe-Phe-NHNH₂ (SEQ ID NO: 51).

10. A method of treating pain in a human subject in need thereof, the method comprising administering to the human subject a composition comprising a therapeutically effective amount of endomorphin-2 or a tetrapeptide derivative thereof of formula (I):
R₁-Tyr-Pro-Phe-Phe-R₂ formula (I)
or a pharmaceutically acceptable salt thereof, and optionally a pharmaceutically or cosmetically acceptable carrier, wherein R₁ is an acyl radical having up to 29 carbon atoms; R₂ is NHR₃ or NHNHR₄, or alternatively the carboxy terminus —COR₂ is CN; R₃ is H, OH, an alkyl, aralkyl or aryl radical having up to 9 carbon atoms; and R₄ is H, an alkyl, aralkyl, aryl or acyl radical having up to 9 carbon atoms.

11. The method of claim 10, wherein the pain is associated with a disease, disorder, condition symptom, or syndrome selected from the group consisting of arthritis, headache, migraine headache, hangover headache, dental pain, lipoma, muscle pain, pharyngitis, sprain, trauma, sunburn, thermal burn, viral infection, herpes zoster, wounds, post-operative sites and injection sites.

12. The method of claim 11, wherein the pain is associated with arthritis, headache, muscles or joints.

13. The method of claim 12, wherein the arthritis is osteoarthritis, psoriatic arthritis, or rheumatoid arthritis.

14. The method of claim 10, wherein the composition comprises at least 0.2% by weight or volume of endomorphin-2 or the tetrapeptide derivative thereof of formula (I).

15. The method of claim 10, wherein the composition is administered topically.

16. The method of claim 10, wherein the composition is administered systemically.

17. The method of claim 10, wherein wherein R₁ is selected from the group consisting of H, Ab, Ac, Ba, Bo, Bz, Fo, Hd, He, Hp, Ip, Le, Ln, Na, Np, Oa, Pa, Pc, Pe and Pg; and R₂ is selected from the group consisting of NH₂, NHCH₃, NHCH₂CH₃, NHCH₂CH₂CH₃; NHCH(CH₃)₂; NHC₆H₅; NHCH₂C₆H₅; NHNH₂; NHNHCH₃; NHNHCH₂CH₃; NHNHCH₂CH₂CH₃; NHNHCH(CH₃)₂; NHNHC₆H₅; NHNHCH₂C₆H₅; NHNHAc; NHNHPa; NHNHBz; NHNHOa; NHNHFo; and NHOH.

18. The method of claim 10, wherein endomorphin-2 or the tetrapeptide derivative thereof of formula (I) is selected from the group consisting of: Tyr-Pro-Phe-Phe-NH₂ (SEQ ID NO: 1); N-Ab-Tyr-Pro-Phe-Phe-NH₂ (SEQ ID NO: 124); N-Ac-Tyr-Pro-Phe-Phe-NH₂ (SEQ ID NO: 23); N-Ba-Tyr-Pro-Phe-Phe-NH₂ (SEQ ID NO: 202); N-Bo-Tyr-Pro-Phe-Phe-NH₂ (SEQ ID NO: 144); N-Bz-Tyr-Pro-Phe-Phe-NH₂ (SEQ ID NO: 84); N-Fo-Tyr-Pro-Phe-Phe-NH₂ (SEQ ID NO: 104); N-Hd-Tyr-Pro-Phe-Phe-NH₂ (SEQ ID NO: 164); N-He-Tyr-Pro-Phe-Phe-NH₂ (SEQ ID NO: 204); N-Hp-Tyr-Pro-Phe-Phe-NH₂ (SEQ ID NO: 205); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Na-Tyr-Pro-Phe-Phe-NH₂ (SEQ ID NO: 207); N-Np-Tyr-Pro-Phe-Phe-NH₂ (SEQ ID NO: 223); N-Oa-Tyr-Pro-Phe-Phe-NH₂ (SEQ ID NO: 205); N-Pa-Tyr-Pro-Phe-Phe-NH₂ (SEQ ID NO: 64); N-Pc-Tyr-Pro-Phe-Phe-NH₂ (SEQ ID NO: 183); N-Pe-Tyr-Pro-Phe-Phe-NH₂ (SEQ ID NO: 203); N-Pg-Tyr-Pro-Phe-Phe-NH₂ (SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Ba-Tyr-Pro-Phe-Phe-NHNH₂ (SEQ ID NO: 208); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Fo-Tyr-Pro-Phe-Phe-NHNH₂ (SEQ ID NO: 111); N-Hd-Tyr-Pro-Phe-Phe-NHNH₂ (SEQ ID NO: 172); N-He-Tyr-Pro-Phe-Phe-NHNH₂ (SEQ ID NO: 210); N-Hp-Tyr-Pro-Phe-Phe-NHNH₂ (SEQ ID NO: 211); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂(SEQ ID NO: 212); N-Pa-Tyr-Pro-Phe-Phe-NHNH₂ (SEQ ID NO: 71); N-Pc-Tyr-Pro-Phe-Phe-NHNH₂ (SEQ ID NO: 191); N-Pe-Tyr-Pro-Phe-Phe-NHNH₂ (SEQ ID NO: 209); N-Pg-Tyr-Pro-Phe-Phe-NHNH₂ (SEQ ID NO: 51); Tyr-Pro-Phe-Phe-NHNHAc (SEQ ID NO: 17); N-Ab-Tyr-Pro-Phe-Phe-NHNHAc (SEQ ID NO: 138); N-Ac-Tyr-Pro-Phe-Phe-NHNHAc (SEQ ID NO: 38); N-Ba-Tyr-Pro-Phe-Phe-NHNHAc (SEQ ID NO: 228); N-Bo-Tyr-Pro-Phe-Phe-NHNHAc (SEQ ID NO: 158); N-Bz-Tyr-Pro-Phe-Phe-NHNHAc (SEQ ID NO: 98); N-Fo-Tyr-Pro-Phe-Phe-NHNHAc (SEQ ID NO: 118); N-Hd-Tyr-Pro-Phe-Phe-NHNHAc (SEQ ID NO: 179); N-He-Tyr-Pro-Phe-Phe-NHNHAc (SEQ ID NO: 229); N-Hp-Tyr-Pro-Phe-Phe-NHNHAc (SEQ ID NO: 230); N-Ip-Tyr-Pro-Phe-Phe-NHNHAc (SEQ ID NO: 231); N-Le-Tyr-Pro-Phe-Phe-NHNHAc (SEQ ID NO: 232); N-Ln-Tyr-Pro-Phe-Phe-NHNHAc (SEQ ID NO: 233); N-Na-Tyr-Pro-Phe-Phe-NHNHAc (SEQ ID NO: 234); N-Np-Tyr-Pro-Phe-Phe-NHNHAc (SEQ ID NO: 235); N-Oa-Tyr-Pro-Phe-Phe-NHNHAc (SEQ ID NO: 236); N-Pa-Tyr-Pro-Phe-Phe-NHNHAc (SEQ ID NO: 78); N-Pc-Tyr-Pro-Phe-Phe-NHNHAc (SEQ ID NO: 198); N-Pe-Tyr-Pro-Phe-Phe-NHNHAc (SEQ ID NO: 237); N-Pg-Tyr-Pro-Phe-Phe-NHNHAc (SEQ ID NO: 198); Tyr-Pro-Phe-Phe-CN (SEQ ID NO: 238); N-Ab-Tyr-Pro-Phe-Phe-CN (SEQ ID NO: 239); N-Ac-Tyr-Pro-Phe-Phe-CN (SEQ ID NO: 240); N-Ba-Tyr-Pro-Phe-Phe-CN (SEQ ID NO: 241); N-Bo-Tyr-Pro-Phe-Phe-CN (SEQ ID NO: 242); N-Bz-Tyr-Pro-Phe-Phe-CN (SEQ ID NO: 243); N-Fo-Tyr-Pro-Phe-Phe-CN (SEQ ID NO: 244); N-Hd-Tyr-Pro-Phe-Phe-CN (SEQ ID NO: 245); N-He-Tyr-Pro-Phe-Phe-CN (SEQ ID NO: 246); N-Hp-Tyr-Pro-Phe-Phe-CN (SEQ ID NO: 247); N-Ip-Tyr-Pro-Phe-Phe-CN (SEQ ID NO: 248); N-Le-Tyr-Pro-Phe-Phe-CN (SEQ ID NO: 249); N-Ln-Tyr-Pro-Phe-Phe-CN (SEQ ID NO: 250); N-Na-Tyr-Pro-Phe-Phe-CN (SEQ ID NO: 251); N-Np-Tyr-Pro-Phe-Phe-CN (SEQ ID NO: 252); N-Oa-Tyr-Pro-Phe-Phe-CN (SEQ ID NO: 253); N-Pa-Tyr-Pro-Phe-Phe-CN (SEQ ID NO: 254); N-Pc-Tyr-Pro-Phe-Phe-CN (SEQ ID NO: 255); N-Pe-Tyr-Pro-Phe-Phe-CN (SEQ ID NO: 256); and N-Pg-Tyr-Pro-Phe-Phe-CN (SEQ ID NO: 257).

19. The method of claim 10, wherein endomorphin-2 or the tetrapeptide derivative thereof of formula (I) is selected from the group consisting of: endomorphin-2 or the tetrapeptide derivative thereof of formula (I) is selected from the group consisting of: N-Ab-Tyr-Pro-Phe-Phe-NH₂(SEQ ID NO: 124); N-Ac-Tyr-Pro-Phe-Phe-NH₂ (SEQ ID NO: 23); N-Bo-Tyr-Pro-Phe-Phe-NH₂ (SEQ ID NO: 144); N-Bz-Tyr-Pro-Phe-Phe-NH₂ (SEQ ID NO: 84); N-Hd-Tyr-Pro-Phe-Phe-NH₂ (SEQ ID NO: 164); N-Ip-Tyr-Pro-Phe-Phe-NH₂ (SEQ ID NO: 220); N-Le-Tyr-Pro-Phe-Phe-NH₂ (SEQ ID NO: 221); N-Ln-Tyr-Pro-Phe-Phe-NH₂ (SEQ ID NO: 222); N-Np-Tyr-Pro-Phe-Phe-NH₂ (SEQ ID NO: 223); N-Oa-Tyr-Pro-Phe-Phe-NH₂ (SEQ ID NO: 205); N-Pc-Tyr-Pro-Phe-Phe-NH₂ (SEQ ID NO: 183); N-Pg-Tyr-Pro-Phe-Phe-NH₂(SEQ ID NO: 44); Tyr-Pro-Phe-Phe-NHNH₂ (SEQ ID NO: 10); N-Ab-Tyr-Pro-Phe-Phe-NHNH₂ (SEQ ID NO: 131); N-Ac-Tyr-Pro-Phe-Phe-NHNH₂ (SEQ ID NO: 31); N-Bo-Tyr-Pro-Phe-Phe-NHNH₂ (SEQ ID NO: 151); N-Bz-Tyr-Pro-Phe-Phe-NHNH₂ (SEQ ID NO: 91); N-Hd-Tyr-Pro-Phe-Phe-NHNH₂ (SEQ ID NO: 172); N-Ip-Tyr-Pro-Phe-Phe-NHNH₂ (SEQ ID NO: 224); N-Le-Tyr-Pro-Phe-Phe-NHNH₂ (SEQ ID NO: 225); N-Ln-Tyr-Pro-Phe-Phe-NHNH₂ (SEQ ID NO: 226); N-Na-Tyr-Pro-Phe-Phe-NHNH₂ (SEQ ID NO: 213); N-Np-Tyr-Pro-Phe-Phe-NHNH₂ (SEQ ID NO: 227); N-Oa-Tyr-Pro-Phe-Phe-NHNH₂(SEQ ID NO: 212); N-Pc-Tyr-Pro-Phe-Phe-NHNH₂ (SEQ ID NO: 191); and N-Pg-Tyr-Pro-Phe-Phe-NHNH₂ (SEQ ID NO: 51).
